(19) United States
(12) Patent Application Publication
(10) Pub. No.: US 2019/0185820 A1
(43) Pub. Date: Jun. 20, 2019

(54) Compositions and Methods for Inhibiting Stem Cell Aging

(71) Applicant: The Regents of the University of California, Oakland, CA (US)

(72) Inventors: Danica Chen, San Francisco, CA (US); Hanzhi Luo, Berkeley, CA (US)

(21) Appl. No.: 16/319,749

(22) PCT Filed: Aug. 23, 2017
(86) PCT No.: PCT/US17/48248

§ 371 Date: Jan. 22, 2019

Related U.S. Application Data
(60) Provisional application No. 62/380,101, filed on Aug. 26, 2016.

(51) Int. Cl. C12N 5/16; C12N 15/85; C12N 15/113; A61K 35/28; C12N 5/074; A61P 25/28; A61P 21/00; A61K 31/7105; A61K 31/4453

(52) U.S. Cl. CPC C12N 5/16; C12N 15/85; C12N 15/1137; A61K 35/28; A61K 45/06; A61P 25/28; A61P 21/00; A61K 31/7105; A61K 31/4453; C12N 5/0607

(57) ABSTRACT

The present disclosure provides methods of improving the function of stem cells, and/or reducing or inhibiting or inhibiting stem cell aging.

### CROSS-REFERENCE

This application claims the benefit of U.S. Provisional Patent Application No. 62/380,101, filed Aug. 26, 2016, which application is incorporated herein by reference in its entirety.

### INCORPORATION BY REFERENCE OF SEQUENCE LISTING PROVIDED AS A TEXT FILE

A Sequence Listing is provided herewith as a text file, “BERK-341WO_SEQ_LISTING_ST25” created on Aug. 10, 2017 and having a size of 99 KB. The contents of the text file are incorporated by reference herein in their entirety.

### INTRODUCTION

Degeneration and dysfunction of aging tissues are attributable to the deterioration of tissue-specific stem cells. Stem cell aging is thought to be due to cumulative cellular damage, leading to permanent cell cycle arrest and cell death.

There is a need in the art for methods of reducing stem cell aging. There is a need in the art for methods of inhibiting tissue degeneration or injury, and methods of treating tissue degenerative diseases.

### SUMMARY

The present disclosure provides methods of improving the function of stem cells, and/or reducing or inhibiting or reversing stem cell aging.

### BRIEF DESCRIPTION OF THE DRAWINGS

FIG. 1A-1D depict requirement for SIRT2 for hematopoietic stem cell (HSC) maintenance at an old age.

FIG. 2A-2F depict the effect of SIRT2 on pyroptosis in aged HSCs.

FIG. 3A-3F depict the effect of SIRT2 on NLRP3 deacetylation and caspase-1 activation.

FIG. 4A-4H depict the effect of mitochondrial stress-initiated caspase-1-mediated pyroptosis on HSC aging.

FIG. 5A-5C depict reduction of SIRT2 expression with age in HSCs.

FIG. 6A-6D depict lack of requirement for SIRT2 for HSC maintenance at a young age.

FIG. 7 depicts expression of SIRT2 in various hematopoietic cellular compartments in the bone marrow.

FIG. 8A-8B depict the effect of SIRT2 on pyroptosis in aged HSCs.

FIG. 9A-9C depict SIRT2 regulation of HSCs.

FIG. 10A-10C depict requirement for NLRP3 for SIRT2 repression of caspase-1 activation.

FIG. 11A-11B depict lack of effect of SIRT1 on NLRP3 acetylation.

FIG. 12 provides an alignment of NLRP3 amino acid sequences from various species: Mouse (SEQ ID NO:22), Human (SEQ ID NO:23), Monkey (SEQ ID NO:24), Bovine (SEQ ID NO:25), Horse (SEQ ID NO:26), Pig (SEQ ID NO:27), Rat (SEQ ID NO:28), Rabbit (SEQ ID NO:29).

FIG. 13A-13B depict the effect of NLRP3 acetylation on pyroptosome formation.

FIG. 14A-14B depict the effect of NLRP3 deacetylation on the functionality of aged HSCs.

FIG. 15A-15C depict induction of pyroptosis in aged HSCs.

FIG. 16A-16C depict the effect of SIRT2 overexpression on young HSCs.

FIG. 17A-17D depict regulation of HSC aging by caspase-1.

FIG. 18 depicts the effect of SIRT3 and SIRT7 on caspase-3 activity in aged HSCs.

FIG. 19 provides a list of antibodies and reagents in Table 1 and Table 2.

FIG. 20 provides a list of primers in Table 3.

FIG. 21 provides an amino acid sequence of an NLRP3 polypeptide.

FIG. 22 provides an amino acid sequence of an NLRP3 polypeptide.

FIG. 23 provides an amino acid sequence of a SIRT2 polypeptide.

FIG. 24-28 provide amino acid sequences of caspase-1 polypeptides.

### DEFINITIONS

The term “stem cell” is used herein to refer to a cell that has the ability both to self-renew, and to generate differentiated progeny (see Morrison et al. (1997) Cell 88:287-298). Generally, stem cells also have one or more of the following properties: an ability to undergo asynchronous, or asymmetric replication; i.e., where the two daughter cells after division can have different phenotypes; extensive self-renewal capacity; capacity for existence in a mitotically quiescent form; and clonal regeneration of all the tissue in which they exist, for example the ability of hematopoietic stem cells to reconstitute all hematopoietic lineages. “Progenitor cells” differ from stem cells in that they typically do not have the extensive self-renewal capacity, and often can only regenerate a subset of the lineages in the tissue from which they derive.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines, simians, humans, mammalian farm animals, mammalian sport animals, and mammalian pets.

A “therapeutically effective amount” or “efficacious amount” means the amount of a compound that, when administered to a mammal or other subject for treating a disease, is sufficient to effect such treatment for the disease. The “therapeutically effective amount” will vary depending on the compound, the disease and its severity and the age, weight, etc., of the subject to be treated.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle.

As used herein, the phrase “pharmaceutically acceptable carrier” refers to a carrier medium that does not interfere with the effectiveness of the biological activity of the active ingredient. Such a carrier medium is essentially chemically inert and nontoxic.

As used herein, the phrase “pharmaceutically acceptable” means approved by a regulatory agency of the Federal government or a state government, or listed in the U.S. Pharmacopeia or other generally recognized pharmacopeia for use in animals, and more particularly for use in humans.

As used herein, the term “carrier” refers to a diluent, adjuvant, excipient, or vehicle with which the therapeutic is administered. Such carriers can be sterile liquids, such as saline solutions in water, or oils, including those of petroleum, animal, vegetable or synthetic origin, such as peanut oil, soybean oil, mineral oil, sesame oil and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The carrier, if desired, can also contain minor amounts of wetting or emulsifying agents, or pH buffering agents. These pharmaceutical compositions can take the form of solutions, suspensions, emulsion, tablets, pills, capsules, powders, sustained-release formulations and the like. The composition can be formulated as a suppository, with traditional binders and carriers such as triglycerides. Examples of suitable pharmaceutical carriers are described in Remington's Pharmaceutical Sciences by E. W. Martin. Examples of suitable pharmaceutical carriers are a variety of cationic polyamines and lipids, including, but not limited to N-(1(2,3-dioleyloxy)propyl)-N,N,N-trimethylammonium chloride (DOTMA) and diolesylphosphotidylethanolamine (DOPE). Liposomes are suitable carriers for gene therapy uses of the present disclosure. Such pharmaceutical compositions should contain a therapeutically effective amount of the compound, together with a suitable amount of carrier so as to provide the form for proper administration to the subject. The formulation should suit the mode of administration.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

The terms “nucleic acid” and “polynucleotide” are used interchangeably herein, and refer to a polymeric form of nucleotides of any length, either deoxyribonucleotides or ribonucleotides, or analogs thereof. Non-limiting examples of nucleic acids and polynucleotides include linear and circular nucleic acids, messenger RNA (mRNA), cDNA, recombinant polynucleotides, vectors, probes, primers, single-, double-, or multi-stranded DNA or RNA, genomic DNA, DNA-RNA hybrids, chemically or biochemically modified, non-natural, or derivatized nucleotide bases, oligonucleotides containing modified or non-natural nucleotide bases (e.g., locked-nucleic acids (LNA) oligonucleotides), and interfering RNAs.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence similarity can be determined in a number of different manners. To determine sequence identity, sequences can be aligned using the methods and computer programs, including BLAST, available over the world wide web at ncbi(dot)nlm(dot)nih(dot)gov/BLAST. See, e.g., Altschul et al. (1990), J. Mol. Biol. 215:403-10. Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

The terms “double stranded RNA,” “dsRNA,” “partial-length dsRNA,” “full-length dsRNA,” “synthetic dsRNA,” “in vitro produced dsRNA,” “in vivo produced dsRNA,” “bacterially produced dsRNA,” “isolated dsRNA,” and “purified dsRNA” as used herein refer to nucleic acid molecules capable of being processed to produce a smaller nucleic acid, e.g., a short interfering RNA (siRNA), capable of inhibiting or down regulating gene expression, for example by mediating RNA interference “RNAi” or gene silencing in a sequence-specific manner. Design of a dsRNA or a construct comprising a dsRNA targeted to a gene of interest is routine in the art, see e.g., Timmons et al. (2001) Gene, 263:103-112; Newmark et al. (2003) Proc Natl Acad Sci USA, 100 Supp 1:11861-5; Reddien et al. (2005) Developmental Cell, 8:635-649; Chuang & Meyerowitz (2000) Proc Natl Acad Sci USA, 97:4985-90; Piccin et al. (2001) Nucleic Acid Res, 29:E55-5; Kondo et al. (2006) Genes Genet Syst, 81:129-34; and Lu et al. (2009) FEBS J, 276:3110-23; the disclosures of which are incorporated herein by reference.

The terms “short interfering RNA”, “siRNA”, and “short interfering nucleic acid” are used interchangeably may refer to short hairpin RNA (shRNA), short interfering oligonucleotide, short interfering nucleic acid, short interfering modified oligonucleotide, chemically-modified siRNA, post-transcriptional gene silencing RNA (ptgsRNA), and other short oligonucleotides useful in mediating an RNAi response. In some instances siRNA may be encoded from DNA comprising a siRNA sequence in vitro or in vivo as described herein. When a particular siRNA is described herein, it will be clear to the ordinary skilled artisan as to where and when a different but equivalently effective interfering nucleic acid may be substituted, e.g., the substation of a short interfering oligonucleotide for a described shRNA and the like.

“Complementary,” as used herein, refers to the capacity for precise pairing between two nucleotides of a polynucleotide (e.g., an antisense polynucleotide) and its corresponding target polynucleotide. For example, if a nucleotide at a particular position of a polynucleotide is capable of hydrogen bonding with a nucleotide at a particular position of a target nucleic acid, then the position of hydrogen bonding between the polynucleotide and the target polynucleotide is considered to be a complementary position. The polynucleotide and the target polynucleotide are complementary to each other when a sufficient number of complementary positions in each molecule are occupied by nucleotides that can hydrogen bond with each other. Thus, “specifically hybridizable” and “complementary” are terms which are used to indicate a sufficient degree of precise pairing or complementarity over a sufficient number of nucleotides such that stable and specific binding occurs between the polynucleotide and a target polynucleotide.

It is understood in the art that the sequence of polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a loop structure or hairpin structure). A polynucleotide can comprise at least 70%, at least 80%, at least 90%, at least 95%, at least 99%, or 100% sequence complementarity to a target region within the target nucleic acid sequence to which they are targeted. For example, an antisense nucleic acid in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. As such, an antisense polynucleotide which is 18 nucleotides in length having 4 (four) noncomplementary nucleotides which are flanked by two regions of complete complementarity with the target nucleic acid would have 77.8% overall complementarity with the target nucleic acid. Percent complementarity of an oligomeric compound with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a stem cell” includes a plurality of such stem cells and reference to “the NLPR3 polypeptide” includes reference to one or more NLPR3 polypeptides and equivalents thereof known to those skilled in the art, and so forth. It is further noted that the claims may be drafted to exclude any optional element.

As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides methods of improving the function of stem cells, and/or reducing or inhibiting or reversing stem cell aging. The methods generally involve modulating the level and/or activity of a target gene product (an mRNA; a polypeptide) in an adult stem cell, where the modulating results in inhibition or reversal of aging of the stem cell.

In some cases, a method of the present disclosure reduces or inhibits stem cell aging by at least 10%, at least 15%, at least 20%, at least 30%, at least 35%, at least 40%, at least 45%, at least 50%, at least 55%, at least 60%, at least 65%, at least 70%, at least 75%, or more than 75%, compared to the aging of the adult stem cell not treated with the method. In some cases, a method of the present disclosure reverses stem cell aging.

In some cases, a method of the present disclosure increases the capacity of an adult stem cell to give rise to terminally differentiated cells. In some cases, a method of the present disclosure increases the self-renewal capacity of an adult stem cell.

In some cases, a method of the present disclosure pyroptosome formation in a stem cell by at least 10%, at least 15%, at least 20%, at least 30%, at least 35%, at least 40%, at least 45%, at least 50%, at least 55%, at least 60%, at least 65%, at least 70%, at least 75%, or more than 75%, compared to pyroptosome formation in the stem cell not treated with the method.

In some cases, a method of the present disclosure comprises increasing the activity and/or the level of SIRT2 in an adult stem cell. For example, in some cases, a method of the present disclosure comprises increasing the level of a SIRT2 polypeptide in an adult stem cell. For example, in some cases, a method of the present disclosure comprises increasing the level (amount) of a SIRT2 polypeptide in an adult stem cell by at least about 10%, at least about 25%, at least about 50%, at least about 75%, at least about 100% (or 2-fold), at least about 2.5-fold, at least about 3-fold, at least about 4-fold, at least about 5-fold, or more than 5-fold, compared to the level of a SIRT2 polypeptide in a control adult stem cell not subjected to a method of the present disclosure.

As another example, in some cases, a method of the present disclosure comprises increasing the activity of a SIRT2 polypeptide in an adult stem cell. For example, in some cases, a method of the present disclosure comprises increasing the deacetylase activity of a SIRT2 polypeptide in an adult stem cell by at least about 10%, at least about 25%, at least about 50%, at least about 75%, at least about 100% (or 2-fold), at least about 2.5-fold, at least about 3-fold, at least about 4-fold, at least about 5-fold, or more than 5-fold, compared to the level of deacetylase activity of the SIRT2 polypeptide in a control adult stem cell not subjected to a method of the present disclosure.

As another example, in some cases, a method of the present disclosure comprises reducing the level of an NLRP3 polypeptide in an adult stem cell. For example, in some cases, a method of the present disclosure comprises reducing the level of an NLRP3 polypeptide in an adult stem cell by at least 10%, at least 15%, at least 20%, at least 30%, at least 35%, at least 40%, at least 45%, at least 50%, at least 55%, at least 60%, at least 65%, at least 70%, at least 75%, or more than 75%, compared to the level of the NLRP3 polypeptide in a control adult stem cell not subjected to a method of the present disclosure.

In some cases, a method of the present disclosure comprises reducing the level of acetylated NLRP3 polypeptide in an adult stem cell. For example, in some cases, a method of the present disclosure comprises reducing the level of acetylated NLRP3 polypeptide in an adult stem cell by at least 10%, at least 15%, at least 20%, at least 30%, at least 35%, at least 40%, at least 45%, at least 50%, at least 55%, at least 60%, at least 65%, at least 70%, at least 75%, or more than 75%, compared to the level of acetylated NLRP3 polypeptide in a control adult stem cell not subjected to a method of the present disclosure.

As another example, in some cases, a method of the present disclosure comprises reducing the level of a caspase-1 polypeptide in an adult stem cell. For example, in some cases, a method of the present disclosure comprises reducing the level of a caspase-1 polypeptide in an adult stem cell by at least 10%, at least 15%, at least 20%, at least 30%, at least 35%, at least 40%, at least 45%, at least 50%, at least 55%, at least 60%, at least 65%, at least 70%, at least 75%, or more than 75%, compared to the level of the caspase-1 polypeptide in a control adult stem cell not subjected to a method of the present disclosure.

In some cases, a method of the present disclosure comprises reducing the activity of a caspase-1 polypeptide in an adult stem cell. For example, in some cases, a method of the present disclosure comprises reducing the activity of a caspase-1 polypeptide in an adult stem cell by at least 10%, at least 15%, at least 20%, at least 30%, at least 35%, at least 40%, at least 45%, at least 50%, at least 55%, at least 60%, at least 65%, at least 70%, at least 75%, or more than 75%, compared to the caspase-1 activity in a control adult stem cell not subjected to a method of the present disclosure.

### Stem Cells

Stem cells include adult stem cells. Adult stem cells are also referred to as somatic stem cells.

Adult stem cells are resident in differentiated tissue, but retain the properties of self-renewal and ability to give rise to multiple cell types, usually cell types typical of the tissue in which the stem cells are found. Numerous examples of somatic stem cells are known to those of skill in the art, including muscle stem cells; hematopoietic stem cells; epithelial stem cells; neural stem cells; mesenchymal stem cells; mammary stem cells; intestinal stem cells; mesodermal stem cells; endothelial stem cells; olfactory stem cells; neural crest stem cells; and the like.

Stem cells of interest include mammalian stem cells, where the term “mammalian” refers to any animal classified as a mammal, including humans; non-human primates; domestic and farm animals; and zoo, laboratory, sports, or pet animals, such as dogs, horses, cats, cows, mice, rats, rabbits, etc. In some cases, the stem cell is a human stem cell. In some cases, the stem cell is a rodent (e.g., a mouse; a rat) stem cell. In some cases, the stem cell is a non-human primate stem cell.

Stem cells can express one or more stem cell markers, e.g., SOX9, KRT19, KRT7, LGR5, CA9, FXYD2, CDH6, CLDN18, TSPAN8, BPIFB1, OLFM4, CDH17, and PPARGC1A.

In some embodiments, the stem cell is a hematopoietic stem cell (HSC). HSCs are mesoderm-derived cells that can be isolated from bone marrow, blood, cord blood, fetal liver and yolk sac. HSCs are characterized as CD34⁺ and CD3. HSCs can repopulate the erythroid, neutrophil-macrophage, megakaryocyte and lymphoid hematopoietic cell lineages in vivo. In vitro, HSCs can be induced to undergo at least some self-renewing cell divisions and can be induced to differentiate to the same lineages as is seen in vivo. As such, HSCs can be induced to differentiate into one or more of erythroid cells, megakaryocytes, neutrophils, macrophages, and lymphoid cells.

In other embodiments, the stem cell is a neural stem cell (NSC). Neural stem cells (NSCs) are capable of differentiating into neurons, and glia (including oligodendrocytes, and astrocytes). A neural stem cell is a multipotent stem cell which is capable of multiple divisions, and under specific conditions can produce daughter cells which are neural stem cells, or neural progenitor cells that can be neuroblasts or glioblasts, e.g., cells committed to become one or more types of neurons and glial cells respectively. Methods of obtaining NSCs are known in the art.

In other embodiments, the stem cell is a mesenchymal stem cell (MSC). MSCs originally derived from the embryonal mesoderm and isolated from adult bone marrow, can differentiate to form muscle, bone, cartilage, fat, marrow stroma, and tendon. Methods of isolating MSC are known in the art; and any known method can be used to obtain MSC. See, e.g., U.S. Pat. No. 5,736,396, which describes isolation of human MSC.

Stem cells of interest include aged adult stem cells. For example, in some cases, an aged adult stem cell is an adult stem cell obtained from, or present in, a human individual greater than 50 years, greater than 55 years, greater than 60 years, greater than 65 years, greater than 70 years, greater than 75 years, greater than 80 years, greater than 85 years, or greater than 90 years of age. In some cases, an aged adult stem cell is an adult stem cell obtained from, or present in, a human individual who is from 50 years to 55 years, from 55 years to 60 years, from 60 years to 65 years, from 65 years to 70 years, from 70 years to 75 years, from 75 years to 80 years, from 80 years to 85 years, or from 85 years to 90 years of age.

### Nucleic Acids Encoding SIRT2

In some cases, as described above, a method of the present disclosure comprises increasing the level (amount) of a SIRT2 polypeptide in an adult stem cell by at least about 10%, at least about 25%, at least about 50%, at least about 75%, at least about 100% (or 2-fold), at least about 2.5-fold, at least about 3-fold, at least about 4-fold, at least about 5-fold, or more than 5-fold, compared to the level of a SIRT2 polypeptide in a control adult stem cell not subjected to a method of the present disclosure. A nucleic acid comprising a nucleotide sequence encoding a SIRT2 polypeptide can be introduced into a stem cell, where the encoded SIRT2 polypeptide is produced in the stem cell, thereby increasing the amount of SIRT2 in the stem cell.

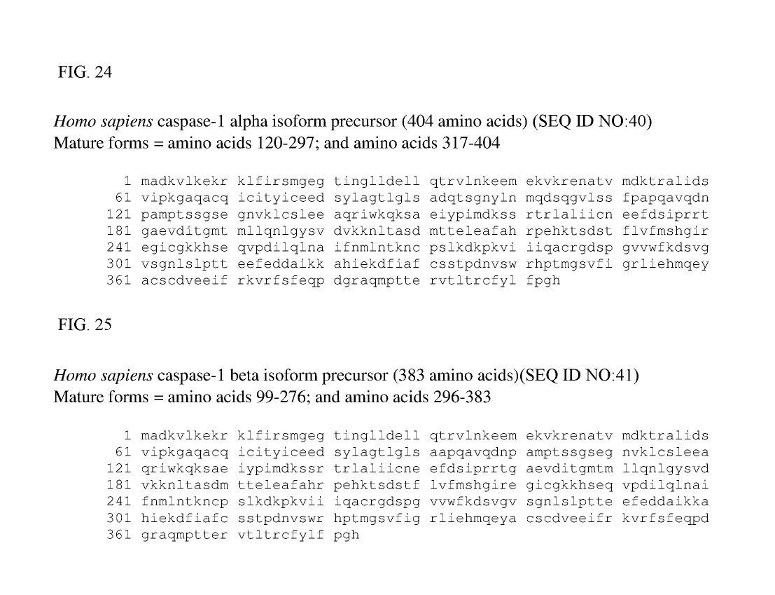

A SIRT2 polypeptide can comprise an amino acid sequence having at least 75%, at least 80%, at least 85%, at least 90%, at least 95%, at least 98%, at least 99%, or 100%, amino acid sequence identity to the SIRT2 amino acid sequence depicted in FIG. 23.

In some cases, the nucleic acid is a recombinant expression vector. Suitable recombinant expression vectors include, but are not limited to, a recombinant retroviral vector, a recombinant lentiviral vector, a recombinant adeno-associate viral vector, a recombinant herpes simplex virus vector, and the like.

In some cases, the nucleotide sequence encoding the SIRT2 polypeptide is operably linked to a promoter, e.g., a promoter that is functional in a mammalian cell. In some cases, the promoter is regulatable (e.g., inducible). In some cases, the promoter is constitutively active.

Suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters.

### Interfering Nucleic Acid

In some cases, as described above, a method of the present disclosure comprises reducing the level of an NLRP3 polypeptide in an adult stem cell. In some cases, as described above, a method of the present disclosure comprises reducing the level of a caspase-1 polypeptide in an adult stem cell. The present disclosure provides interfering nucleic acids, and compositions comprising such interfering nucleic acids, which interfering nucleic acids reduce the level of a polypeptide such as an NLRP3 polypeptide or a caspase-1 polypeptide. Given the availability of NLRP3 amino acid sequences, and caspase-1 amino acid sequences, and given the availability of nucleotide sequences of nucleic acids encoding NLRP3 or caspase-1, those skilled in the art can readily design interfering nucleic acid that would reduce the level of NLRP3 or caspase-1 polypeptide in a stem cell.

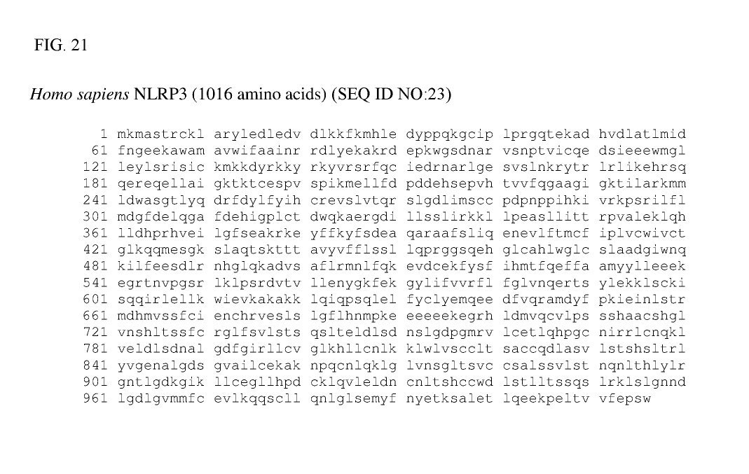

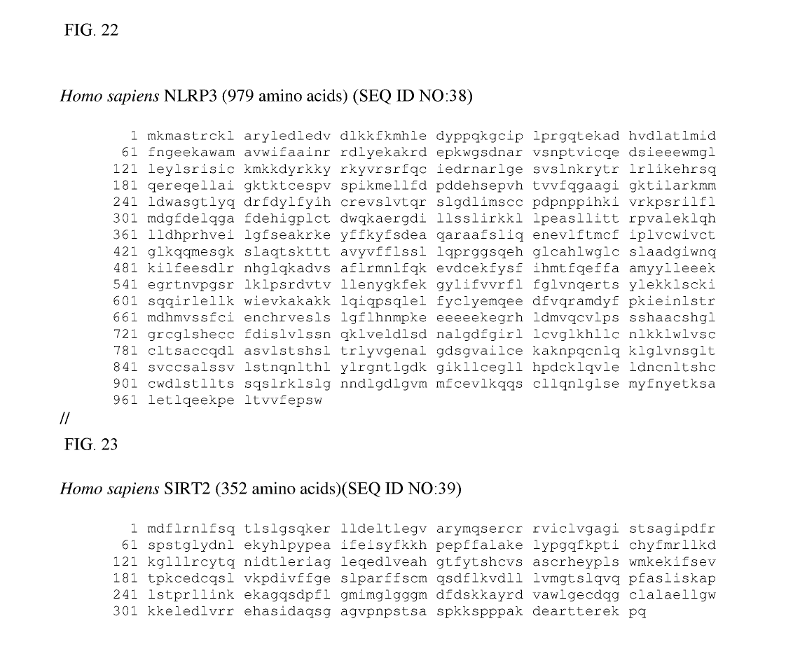

An NLRP3 polypeptide can comprise an amino acid sequence having at least 75%, at least 80%, at least 85%, at least 90%, at least 95%, at least 98%, at least 99%, or 100%, amino acid sequence identity to the NLRP3 amino acid sequence depicted in FIG. 21. An NLRP3 polypeptide can comprise an amino acid sequence having at least 75%, at least 80%, at least 85%, at least 90%, at least 95%, at least 98%, at least 99%, or 100%, amino acid sequence identity to the NLRP3 amino acid sequence depicted in FIG. 22.

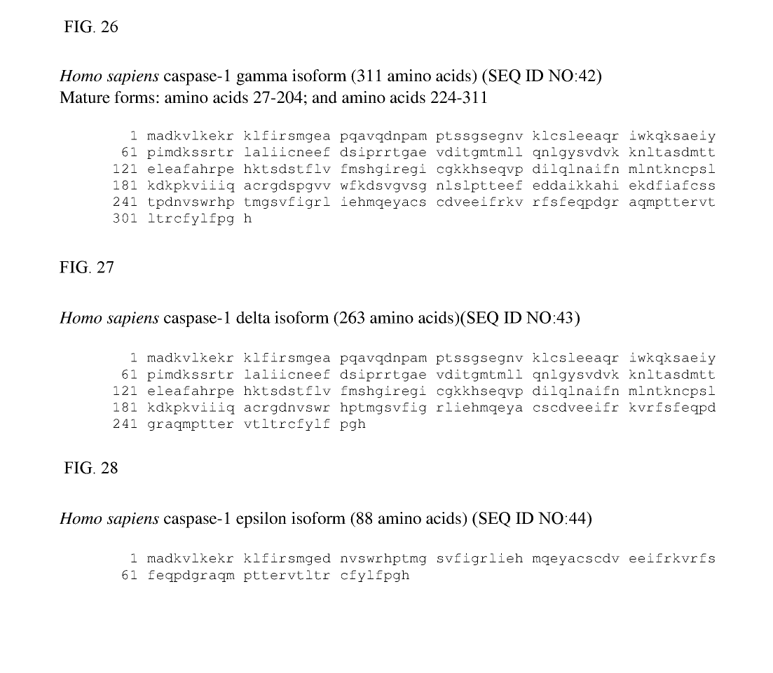

A caspase-1 polypeptide can comprise an amino acid sequence having at least 75%, at least 80%, at least 85%, at least 90%, at least 95%, at least 98%, at least 99%, or 100%, amino acid sequence identity to the caspase-1 amino acid sequence depicted in FIG. 24. A caspase-1 polypeptide can comprise an amino acid sequence having at least 75%, at least 80%, at least 85%, at least 90%, at least 95%, at least 98%, at least 99%, or 100%, amino acid sequence identity to the caspase-1 amino acid sequence depicted in FIG. 25. A caspase-1 polypeptide can comprise an amino acid sequence having at least 75%, at least 80%, at least 85%, at least 90%, at least 95%, at least 98%, at least 99%, or 100%, amino acid sequence identity to the caspase-1 amino acid sequence depicted in FIG. 26. A caspase-1 polypeptide can comprise an amino acid sequence having at least 75%, at least 80%, at least 85%, at least 90%, at least 95%, at least 98%, at least 99%, or 100%, amino acid sequence identity to the caspase-1 amino acid sequence depicted in FIG. 27. A caspase-1 polypeptide can comprise an amino acid sequence having at least 75%, at least 80%, at least 85%, at least 90%, at least 95%, at least 98%, at least 99%, or 100%, amino acid sequence identity to the caspase-1 amino acid sequence depicted in FIG. 28.

The terms “double stranded RNA,” “dsRNA,” “partial-length dsRNA,” “full-length dsRNA,” “synthetic dsRNA,” “in vitro produced dsRNA,” “in vivo produced dsRNA,” “bacterially produced dsRNA,” “isolated dsRNA,” and “purified dsRNA” as used herein refer to nucleic acid molecules capable of being processed to produce a smaller nucleic acid, e.g., a short interfering RNA (siRNA), capable of inhibiting or down regulating gene expression, for example by mediating RNA interference “RNAi” or gene silencing in a sequence-specific manner. Design of a dsRNA or a construct comprising a dsRNA targeted to a gene of interest is routine in the art, see e.g., Timmons et al. (2001) Gene, 263:103-112; Newmark et al. (2003) Proc Natl Acad Sci USA, 100 Supp 1:11861-5; Reddien et al. (2005) Developmental Cell, 8:635-649; Chuang & Meyerowitz (2000) Proc Natl Acad Sci USA, 97:4985-90; Piccin et al. (2001) Nucleic Acid Res, 29:E55-5; Kondo et al. (2006) Genes Genet Syst, 81:129-34; and Lu et al. (2009) FEBS J, 276:3110-23; the disclosures of which are incorporated herein by reference.

dsRNA may be produced de novo or may be produced from “dsRNA templates”, i.e., nucleic acid templates for generating a dsRNA targeted to a particular gene. A dsRNA template or a construct for generating a dsRNA targeted to a particular gene are obtained by any convenient method and need not necessarily be comprised of RNA, e.g., a dsRNA template may be DNA, e.g., single stranded DNA or double stranded DNA. dsRNA templates may be obtained by generating a copy of a naturally occurring spliced mRNA, e.g., a cDNA, using molecular techniques, e.g., reverse transcription or first strand synthesis. dsRNA templates may also be obtained by producing a copy of the coding region, e.g. the CDS, of a gene sequence obtained from sequencing data, e.g., publicly available databases of transcriptome and genomic sequences (see e.g., genomic information from the National Center for Biotechnology Information (NCBI) available on the internet at www(dot)ncbi(dot)nlm(dot)nih(dot)gov/genome/browse/; transcriptome information at the Exon Bioinformatics for Discovery available at http://exon(dot)niaid(dot)nih(dot)gov/transcriptome(dot)html; Zeng & Extavour (2012) Database, bas048; and Wurm et al. (2009) BMC Genomics, 10:5, the disclosures of which are incorporated herein by reference), de novo sequencing of isolated nucleic acid, predicted gene sequences generated by gene prediction software (e.g., ATGpr, AUGUSTUS, BGF, DIOGENES, Dragon Promoter Finder, EUGENE, FGENESH, FRAMED, GENIUS, geneid, GENEPARSER, GeneMark, GeneTrack, GENOMESCAN, GENSCAN, GLIMMER, GLIMMERHMM, GrainEXP, MORGAN, NIX, NNPP, NNSPLICE, ORF FINDER, Regulatory Sequence Analysis Tool, SPLICEPREDICTOR, VEIL, and the like), and the like. Such first and iterative copies of dsRNA templates may represent the same sequence, e.g., the same sequence in the same 5′ to 3′ orientation as the sequence from which the copy was generated, or may represent the complement, the reverse, or the reverse complement of the sequence from which the copy was generated as methods for producing subsequent copies or modifying sequence orientation are well known in the art. In certain instances, a mRNA or a coding region of a gene is constructed from the genomic locus of a gene by assembly of all or some, e.g., about 1 or more, about 2 or more, about 3 or more, about 4 or more, about half, more than half, about 75% or more, about 80% or more, about 90% or more, of the exons of the genetic locus into a synthetic mRNA sequence or synthetic cDNA sequence and the resulting sequence is used to generate synthetic mRNA or synthetic cDNA. Assembly of exons of a genetic locus is routine in the art and can performed by identifying exon-intron junctions either manually or with the help of software that identifies exon-intron junctions either automatically or through user input.

In certain instances, the dsRNA template is a full-length dsRNA template and therefore the dsRNA generated from the template is a full-length dsRNA. By “full-length dsRNA” is meant a dsRNA that comprises the full length sequence of a gene, e.g., all of the coding exons of a gene, all of the coding exons of a gene including 5′ or 3′ untranslated regions of a gene, all of a gene sequence contained between the start codon of a gene and the stop codon of the same gene, etc. In other cases, the dsRNA template is a full-length dsRNA template but is used only to generate a partial-length dsRNA. By partial dsRNA is meant any dsRNA of a gene that contains fewer than all of the coding exons of a gene, e.g., all of a gene except for a portion of an exon, all of a gene except one exon of the gene, all of a gene except more than one exon of a gene, all of a gene except more than two exons of a gene, all of a gene except more than three exons of a gene, all of a gene except more than four exons of a gene, or all of a gene except more than five exons of a gene. Partial-length dsRNA may also represent a dsRNA that includes only a percent portion of the full-length dsRNA of a particular gene but retains the function of activating gene specific silencing by RNAi, e.g., about 95%, about 90%, about 80%, about 70%, about 60%, about 50%, about 40%, about 30%, about 20%, about 10%, about 5%, about 3%, about 2%, about 1%, about, or less than 1% of a full-length dsRNA of a particular gene. In still other instances a partial-length dsRNA template is used to generate a partial-length dsRNA, i.e., partial-length dsRNA is generated from a partial gene sequence or clone and need not be generated from a full-length sequence or clone.

In certain instances, a dsRNA template is cloned, with or without alteration of the dsRNA template sequence, and cloned or inserted into a vector, e.g., a plasmid or phage DNA, to generate a dsRNA construct. By “alteration of the dsRNA templates sequence” is meant that the dsRNA template sequence is modified either directly by introducing mutations, e.g., point mutations, insertions, deletions, silent mutations, and the like, to the original dsRNA template sequence obtained. Alteration of the dsRNA template sequence may result in a mutated dsRNA template sequence that shares about 95%, about 90%, about 80%, about 70%, about 60%, about 50%, or less than about 50% homology with the original dsRNA template sequence. In other instances, the original dsRNA template sequence obtained may be left unmutated or not mutated and one or more nucleotides may be attached to the ends of the original dsRNA template sequence, or some combination therein. In certain instances, an unmutated dsRNA template is amended with additional nucleotides that contain functional sequences, i.e., sequences that may be used for downstream applications of the dsRNA template, e.g., enzyme recognition sites including polymerase recognition sites or endonuclease recognition sites or recombination sites. Methods of nucleic acid cloning and transform which find use in cloning and transforming dsRNA templates are known in the art; see, e.g., Fire et al. (1990) Gene, 93:189-198; Timmons et al. (2001) Gene, 263:103-112; Newmark et al. (2003) Proc Natl Acad Sci USA, 100 Supp 1:11861-5; Reddien et al. (2005) Developmental Cell, 8:635-649; Chuang & Meyerowitz (2000) Proc Natl Acad Sci USA, 97:4985-90; Piccin et al. (2001) Nucleic Acid Res, 29:E55-5; and Kondo et al. (2006) Genes Genet Syst, 81:129-34; the disclosures of which are incorporated herein by reference.

In some embodiments, the dsRNA template, e.g., dsRNA template inserted into a vector, is transformed into a host cell specifically designed for the production of dsRNA. For example, the host cell into which the dsRNA template is transformed may be a host cell deficient in one or more processes that disrupts the production of dsRNA. In certain embodiments, the host cell is a bacterial strain deficient in an enzyme that cleaves dsRNA, e.g., an RNase enzyme or an RNaseIII enzyme. For example, the host cell may have a mutated RNase gene wherein the RNase gene is mutated by a point mutation, a frameshift mutation, or an insertion mutation. In certain embodiments, the RNase gene of the host cell is mutated by insertional mutagenesis by insertion of a polynucleotide into the coding region of the RNase gene such that the presence of the polynucleotide, and thus the presence of mutated RNase, may be selected for. In certain embodiments, the host cell is a bacterial strain with an RNaseIII gene mutated by insertion of an antibiotic resistance gene, e.g., a tetracycline gene, into the coding region of the RNaseIII, e.g., a HT115 bacterial strain, see, e.g., Timmons et al. (2001) Gene, 263:103-112, the disclose of which is incorporated by reference herein.

In certain instances, a dsRNA construct, e.g., a cloned dsRNA or a cloned dsRNA template that has been introduced into a vector, e.g., a plasmid or phage DNA, is used to generate dsRNA. dsRNA constructs, e.g., dsRNA plasmid constructs, may be used to generate in vitro transcribed dsRNA through the use of an in vitro transcription reaction, e.g., through the use of an in vitro transcription kit or a dsRNA synthesis kit, non-limiting examples of commercially available in vitro transcription kits and dsRNA synthesis kit include MEGAscript® RNAi Kits (Life Technologies, Grand Island, N.Y.), Replicator RNAi Kits (Thermo Scientific®, a division of Fisher Scientific®, Pittsburgh, Pa.), T7 RiboMAX™ (Promega Corporation, Madison, Wis.), MAXIscript® (Life Technologies, Grand Island, N.Y.), T7 High Yield RNA Synthesis Kit (New England Biolabs, Ipswich, Mass.), SP6/T7 Transcription Kit (Roche Applied Science, Indianapolis, Ind.), and the like.

In some instances, suitable nucleic acids, e.g., nucleic acid templates, interfering nucleic acids (e.g., dsRNA), etc., and nucleic acid reagents including those synthetically or recombinantly produced, may be obtained from one or more commercial suppliers or commercial custom synthesis companies, including but not limited to e.g., IBA GmbH (Goettingen, Germany), Eurofins Genomics (Ebersberg, Germany), tebu-bio (Le Perray-en-Yvelines, France), Sigma-Aldrich (St Louis, Mo.), Ambion (Austin, Tex.), Applied Biosystems (Foster City, Calif.), Avecia OligoMedicines (Milford, Mass.), BioCat (Heidelberg, Germany), BioSpring (Frankfurt, Germany), Exiqon (Vedbaek, Denmark), GenScript (Piscataway, N.J.), Gene Tools (Philomath, Oreg.), Imgenex (San Diego, Calif.), Integrated DNA Technologies (Coralville, Iowa), Life Technologies (Grand Island, N.Y.), MWG-Biotech (Ebersberg, Germany), Oligoengine (Seattle, Wash.), QIAGEN (Germantown, Md.), SABiosciences (Frederick, Md.), Sigma-Genosys (The Woodlands, Tex.), and the like.

In certain embodiments of the present disclosure, dsRNA constructs may also be transformed into an organism, e.g., a phage, a virus, a prokaryote, a eukaryote, a bacterium, a yeast, a cell of a cell culture system, a cell of a mammalian cell culture system, and the like, for the purpose of generating dsRNA in vivo. Methods for production of dsRNA in vivo, e.g., by introducing a dsRNA construct into a living cell by transformation of dsRNA constructs, are well known in the art, see, e.g., Timmons et al. (2001) Gene, 263:103-112; Newmark et al. (2003) Proc Natl Acad Sci USA, 100 Supp 1:11861-5; Reddien et al. (2005) Developmental Cell, 8:635-649; U.S. Pat. Nos. 6,506,559; and 7,282,564, the disclosures of which are incorporated herein by reference. In certain instances, the dsRNA construct comprises inducible promoters positioned to allow production of both sense and antisense RNA, e.g. different inducible promoters positioned on both sides of the introduced dsRNA template or the same inducible promoters positioned on both sides of the introduced dsRNA template. Inducible promoters are examples of transcriptional control elements and such transcriptional control elements, as detailed herein, find use in generating dsRNA are well known in the art.

Transcriptional control elements, e.g., promoters, and enhancers, etc. may be operably linked to a dsRNA template to control production of dsRNA either in vitro or in vivo. Such elements may be constitutively active or preferably controllable through the introduction of a stimulus, e.g., an environmental stimulus (e.g., change in temperature, pH, light exposure, and the like), a chemical or biological stimulus (e.g., a small molecule or chemical, a molecular biology reagent that binds to an activator or repressor, and the like). Transcriptional control elements may be bound to a dsRNA template singly or in arrays containing multiple transcriptional control elements, e.g., about 2, about 3, about 4, about 5, or more than 5 transcriptional control elements. In certain embodiments, transcriptional control elements are operably linked, directly or indirectly to both the 5′ and the 3′ ends a dsRNA template and such arrangements may place transcriptional control elements on either side of a dsRNA template such that the elements are arranged in a parallel or antiparallel manner.

Suitable promoter and enhancer elements are known in the art. For expression in a bacterial cell, suitable promoters include, but are not limited to, lacI, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters.

Suitable inducible promoters, including reversible inducible promoters are known in the art. Such inducible promoters may be isolated and derived from many organisms, e.g., eukaryotes and prokaryotes. Modification of inducible promoters derived from a first organism for use in a second organism, e.g., a first prokaryote and a second eukaryote, a first eukaryote and a second a prokaryote, etc., is well known in the art. Such inducible promoters, and systems based on such inducible promoters are known in the art.

In some instances, the locus or construct or transgene containing the suitable promoter is irreversibly switched through the rearrangement of elements of the construct or transgene, in certain instances by induction of an inducible system. Site-specific recombination may render a promoter irreversibly switched and such recombinations typically make use of cofactors, e.g., DNA-binding proteins, DNA-binding sites, site specific recombinases, and the like, that result in a change in the spatial arrangement of a elements, e.g., promoter elements or regulatory elements, and the dsRNA template. Such rearrangement of elements can be performed in eukaryotic cells, mammalian cells, and such methods are well known. Suitable systems for induction of an irreversible switch are well known in the art, e.g., induction of an irreversible switch may make use of a Cre-lox-mediated recombination (see, e.g., Fuhrmann-Benzakein, et al., PNAS (2000) 28:e99, the disclosure of which is incorporated herein by reference). Any suitable combination of recombinase, endonuclease, ligase, recombination sites, etc. known to the art may be used in generating an irreversibly switchable promoter. Methods, mechanisms, and requirements for performing site-specific recombination, described elsewhere herein, find use in generating irreversibly switched promoters and are well known in the art, see, e.g., Grindley et al. (2006) Annual Review of Biochemistry, 567-605 and Tropp (2012) Molecular Biology (Jones & Bartlett Publishers, Sudbury, Mass.), the disclosures of which are incorporated herein by reference.

In certain instances, following generation of dsRNA from a dsRNA construct, e.g., a plasmid, by a cell, e.g., a bacterium or a mammalian cell, the produced dsRNA may be isolated and/or purified according to any convenient method of RNA isolation and/or purification. Isolated and purified dsRNA may also be produced by in vitro methods already discussed. dsRNA, e.g., isolated and purified dsRNA, may be delivered to a target or host organism, unprocessed or without processing of the dsRNA, i.e., dsRNA processing, by any convenient method of introducing the dsRNA described herein or known in the art. In certain instances, dsRNA is processed, e.g., subjected to dsRNA processing or dsRNA in vitro processing, before being introduced into a target or host organism. As used herein, by “dsRNA processing” is meant subjecting a dsRNA to one or more physical forces, one or more chemicals, or one or more enzymes or a combination thereof in order to cleave or digest the dsRNA. Non-limiting examples of enzymes that find use in dsRNA processing include, e.g., a nuclease, a ribonuclease, a RNase, a restriction enzyme, a component of an RNAi processing pathway, e.g., RNase III, Dicer, Drosha, and the like. The skilled artisan will recognize that any convenient method of processing dsRNA may be utilized to generate siRNA as such methods are well known in the art and described below.

In some embodiments, siRNA is produced by methods not requiring the production of dsRNA, e.g., chemical synthesis or de novo synthesis or direct synthesis. Chemically synthesized siRNA may be synthesized on a custom basis or may be synthesized on a non-custom or stock or pre-designed basis.

Custom designed siRNA are routinely available from various manufactures (e.g., Ambion®, a division of Life Technologies®, Grand Island, N.Y.; Thermo Scientific®, a division of Fisher Scientific®, Pittsburgh, Pa.; Sigma-Aldrich®, St. Louis, Mo.; Qiagen®, Hilden, Germany; etc.) which provide access to various tools for the design of siRNA. Tools for the design of siRNA allow for the selection of one or more siRNA nucleotide sequences based on computational programs that apply algorithms on longer input nucleotide sequences to identify candidate siRNA sequences likely to be effective in producing an RNAi effect. Such algorithms can be fully automated or semi-automated, e.g., allowing for user input to guide siRNA selection. Programs applying algorithms for siRNA sequence selection are available remotely on the World Wide Web, e.g., at the websites of manufacturers of chemically synthesized siRNA or at the websites of independent, e.g. open source, developers or at the websites of academic institutions. Programs applying algorithms for siRNA sequence selection may also be obtained, e.g., downloaded or received on compact disk as software. Such programs are well known in the art, see e.g., Naito et al. (2004) Nucleic Acids Research, 32:W124-W129; Boudreau et al. (2013) Nucleic Acids Research, 41:e9; Mysara et al. (2011) PLoS, 6:e25642; and Iyer et al. (2007) Comput Methods Programs Biomed, 85:203-9, which are incorporated herein by reference.

Publicly available tools to facilitate design of siRNAs are available in the art. See, e.g., DEQOR: Design and Quality Control of RNAi (available on the internet at http://deqor(dot)mpi-cbg(dot)de/deqor_new/input(dot)html). See also, Henschel et al. Nucleic Acids Res. 2004 Jul. 1; 32 (Web Server issue):W113-20. DEQOR is a web-based program which uses a scoring system based on state-of-the-art parameters for siRNA design to evaluate the inhibitory potency of siRNAs. DEQOR, therefore, can help to predict (i) regions in a gene that show high silencing capacity based on the base pair composition and (ii) siRNAs with high silencing potential for chemical synthesis. In addition, each siRNA arising from the input query is evaluated for possible cross-silencing activities by performing BLAST searches against the transcriptome or genome of a selected organism. DEQOR can therefore predict the probability that an mRNA fragment will cross-react with other genes in the cell and helps researchers to design experiments to test the specificity of siRNAs or chemically designed siRNAs.

The terms “short interfering nucleic acid,” “siNA,” “short interfering RNA,” “siRNA,” “short interfering nucleic acid molecule,” “short interfering oligonucleotide molecule,” and “chemically-modified short interfering nucleic acid molecule” as used herein refer to any nucleic acid molecule capable of inhibiting or down regulating gene expression, for example by mediating RNA interference “RNAi” or gene silencing in a sequence-specific manner. Design of RNAi molecules, when given a target gene, is routine in the art. See also US 2005/0282188 (which is incorporated herein by reference) as well as references cited therein. See, e.g., Pushparaj et al. Clin Exp Pharmacol Physiol. 2006 33(5-6):504-10; Lutzelberger et al. Handb Exp Pharmacol. 2006 (173):243-59; Aronin et al. Gene Ther. 2006 13(6):509-16; Xie et al. Drug Discov Today. 2006 11(1-2):67-73; Grunweller et al. Curr Med Chem. 2005 12(26):3143-61; and Pekaraik et al. Brain Res Bull. 2005 68(1-2):115-20, the disclosures of which are incorporated herein by reference in their entirety.

Methods for design and production of siRNAs to a desired target are known in the art, and their application to elongase and cuticular hydrocarbon genes for the purposes disclosed herein will be readily apparent to the ordinarily skilled artisan, as are methods of production of siRNAs having modifications (e.g., chemical modifications) to provide for, e.g., enhanced stability, bioavailability, and other properties to enhance use as therapeutics. In addition, methods for formulation and delivery of siRNAs to a subject are also well known in the art. See, e.g., US 2005/0282188; US 2005/0239731; US 2005/0234232; US 2005/0176018; US 2005/0059817; US 2005/0020525; US 2004/0192626; US 2003/0073640; US 2002/0150936; US 2002/0142980; and US2002/0120129, each of which are incorporated herein by reference.

siNA molecules can be of any of a variety of forms. For example the siNA can be a double-stranded polynucleotide molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. siNA can also be assembled from two separate oligonucleotides, where one strand is the sense strand and the other is the antisense strand, wherein the antisense and sense strands are self-complementary. In this embodiment, each strand comprises nucleotide sequence that is complementary to nucleotide sequence in the other strand; such as where the antisense strand and sense strand form a duplex or double stranded structure, for example wherein the double stranded region is about 15 to about 30, e.g., about 15, 16, 17, 18, 19, 20, 21, 22, 23, 24, 25, 26, 27, 28, 29 or 30 base pairs; the antisense strand comprises nucleotide sequence that is complementary to nucleotide sequence in a target nucleic acid molecule or a portion thereof and the sense strand comprises nucleotide sequence corresponding to the target nucleic acid sequence or a portion thereof (e.g., about 15 to about 25 or more nucleotides of the siNA molecule are complementary to the target nucleic acid or a portion thereof).

Alternatively, the siNA can be assembled from a single oligonucleotide, where the self-complementary sense and antisense regions of the siNA are linked by a nucleic acid-based or non-nucleic acid-based linker(s). The siNA can be a polynucleotide with a duplex, asymmetric duplex, hairpin or asymmetric hairpin secondary structure, having self-complementary sense and antisense regions, wherein the antisense region comprises nucleotide sequence that is complementary to nucleotide sequence in a separate target nucleic acid molecule or a portion thereof and the sense region having nucleotide sequence corresponding to the target nucleic acid sequence or a portion thereof.

The siNA can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siNA molecule capable of mediating RNAi. The siNA can also comprise a single stranded polynucleotide having nucleotide sequence complementary to nucleotide sequence in a target nucleic acid molecule or a portion thereof (e.g., where such siNA molecule does not require the presence within the siNA molecule of nucleotide sequence corresponding to the target nucleic acid sequence or a portion thereof), wherein the single stranded polynucleotide can further comprise a terminal phosphate group, such as a 5′-phosphate (see for example Martinez et al., 2002, Cell., 110, 563-574 and Schwarz et al., 2002, Molecular Cell, 10, 537-568, the disclosures of which are incorporated herein by reference in their entirety), or 5′,3′-diphosphate.

In certain embodiments, the siNA molecule contains separate sense and antisense sequences or regions, wherein the sense and antisense regions are covalently linked by nucleotide or non-nucleotide linkers molecules as is known in the art, or are alternately non-covalently linked by ionic interactions, hydrogen bonding, van der Waals interactions, hydrophobic interactions, and/or stacking interactions. In certain embodiments, the siNA molecules comprise nucleotide sequence that is complementary to nucleotide sequence of a target gene. In another embodiment, the siNA molecule interacts with nucleotide sequence of a target gene in a manner that causes inhibition of expression of the target gene.

As used herein, siNA molecules need not be limited to those molecules containing only RNA, but further encompasses chemically-modified nucleotides and non-nucleotides. In certain embodiments, the short interfering nucleic acid molecules of the invention lack 2′-hydroxy (2′-OH) containing nucleotides. siNAs do not necessarily require the presence of nucleotides having a 2′-hydroxy group for mediating RNAi and as such, siNA molecules of the invention optionally do not include any ribonucleotides (e.g., nucleotides having a 2′-OH group). Such siNA molecules that do not require the presence of ribonucleotides within the siNA molecule to support RNAi can however have an attached linker or linkers or other attached or associated groups, moieties, or chains containing one or more nucleotides with 2′-OH groups. Optionally, siNA molecules can comprise ribonucleotides at about 5, 10, 20, 30, 40, or 50% of the nucleotide positions. The modified short interfering nucleic acid molecules of the invention can also be referred to as short interfering modified oligonucleotides “siMON.”

As used herein, the term siNA is meant to be equivalent to other terms used to describe nucleic acid molecules that are capable of mediating sequence specific RNAi, for example short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), and others. In some embodiments, an siNA is an siRNA. In some embodiments, a DNA comprising a nucleotide sequence encoding an siRNA is used. In addition, as used herein, the term RNAi is meant to be equivalent to other terms used to describe sequence specific RNA interference, such as post transcriptional gene silencing, translational inhibition, or epigenetics. For example, siNA molecules of the invention can be used to epigenetically silence a target gene at the post-transcriptional level or the pre-transcriptional level. In a non-limiting example, epigenetic regulation of gene expression by siNA molecules of the invention can result from siNA mediated modification of chromatin structure or methylation pattern to alter gene expression (see, for example, Verdel et al., 2004, Science, 303, 672-676; Pal-Bhadra et al., 2004, Science, 303, 669-672; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237, the disclosures of which are incorporated by reference herein in their entirety).

siNA (e.g., siRNA) molecules contemplated herein can comprise a duplex forming oligonucleotide (DFO) see, e.g., WO 05/019453; and US 2005/0233329, which are incorporated herein by reference). siNA molecules also contemplated herein include multifunctional siNA, (see, e.g., WO 05/019453 and US 2004/0249178).

siNA (e.g., siRNA) molecules contemplated herein can comprise an asymmetric hairpin or asymmetric duplex. By “asymmetric hairpin” as used herein is meant a linear siNA molecule comprising an antisense region, a loop portion that can comprise nucleotides or non-nucleotides, and a sense region that comprises fewer nucleotides than the antisense region to the extent that the sense region has enough complementary nucleotides to base pair with the antisense region and form a duplex with loop. For example, an asymmetric hairpin siNA molecule can comprise an antisense region having length sufficient to mediate RNAi in a cell or in vitro system (e.g. about 15 to about 30, or about 15, 16, 17, 18, 19, 20, 21, 22, 23, 24, 25, 26, 27, 28, 29, or 30 nucleotides) and a loop region comprising about 4 to about 12 (e.g., about 4, 5, 6, 7, 8, 9, 10, 11, or 12) nucleotides, and a sense region having about 3 to about 25 (e.g., about 3, 4, 5, 6, 7, 8, 9, 10, 11, 12, 13, 14, 15, 16, 17, 18, 19, 20, 21, 22, 23, 24, or 25) nucleotides that are complementary to the antisense region. The asymmetric hairpin siNA molecule can also comprise a 5′-terminal phosphate group that can be chemically modified. The loop portion of the asymmetric hairpin siNA molecule can comprise nucleotides, non-nucleotides, linker molecules, or conjugate molecules as described herein.

By “asymmetric duplex” as used herein is meant a siNA molecule having two separate strands comprising a sense region and an antisense region, wherein the sense region comprises fewer nucleotides than the antisense region to the extent that the sense region has enough complementary nucleotides to base pair with the antisense region and form a duplex. For example, an asymmetric duplex siNA molecule of the invention can comprise an antisense region having length sufficient to mediate RNAi in a cell or in vitro system (e.g. about 15 to about 30, or about 15, 16, 17, 18, 19, 20, 21, 22, 23, 24, 25, 26, 27, 28, 29, or 30 nucleotides) and a sense region having about 3 to about 25 (e.g., about 3, 4, 5, 6, 7, 8, 9, 10, 11, 12, 13, 14, 15, 16, 17, 18, 19, 20, 21, 22, 23, 24, or 25) nucleotides that are complementary to the antisense region.

Stability and/or half-life of siRNAs can be improved through chemically synthesizing nucleic acid molecules with modifications (base, sugar and/or phosphate) can prevent their degradation by serum ribonucleases, which can increase their potency (see e.g., Eckstein et al., International Publication No. WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; Gold et al., U.S. Pat. No. 6,300,074; and Burgin et al., supra; all of which are incorporated by reference herein, describing various chemical modifications that can be made to the base, phosphate and/or sugar moieties of the nucleic acid molecules described herein. Modifications that enhance their efficacy in cells, and removal of bases from nucleic acid molecules to shorten oligonucleotide synthesis times and reduce chemical requirements are desired.

For example, oligonucleotides are modified to enhance stability and/or enhance biological activity by modification with nuclease resistant groups, for example, 2′-amino, 2′-C-allyl, 2′-fluoro, 2′-O-methyl, 2′-O-allyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modification of nucleic acid molecules have been extensively described in the art (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci., 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. Ser. No. 60/082,404 which was filed on Apr. 20, 1998; Karpeisky et al., 1998, Tetrahedron Lett., 39, 1131; Eamshaw and Gait, 1998, Biopolymers (Nucleic Acid Sciences), 48, 39-55; Verma and Eckstein, 1998, Annu. Rev. Biochem., 67, 99-134; and Burlina et al., 1997, Bioorg. Med. Chem., 5, 1999-2010; each of which are hereby incorporated in their totality by reference herein). In view of such teachings, similar modifications can be used as described herein to modify the siNA nucleic acid molecules of disclosed herein so long as the ability of siNA to promote RNAi is cells is not significantly inhibited.

Short interfering nucleic acid (siNA) molecules (e.g., siRNA) having chemical modifications that maintain or enhance activity are contemplated herein. Such a nucleic acid is also generally more resistant to nucleases than an unmodified nucleic acid. Accordingly, the in vitro and/or in vivo activity should not be significantly lowered. Nucleic acid molecules delivered exogenously are generally selected to be stable within cells at least for a period sufficient for transcription and/or translation of the target RNA to occur and to provide for modulation of production of the encoded mRNA and/or polypeptide so as to facilitate reduction of the level of the target gene product.

Production of RNA and DNA molecules can be accomplished synthetically and can provide for introduction of nucleotide modifications to provide for enhanced nuclease stability. (see, e.g., Wincott et al., 1995, Nucleic Acids Res. 23, 2677; Caruthers et al., 1992, Methods in Enzymology 211, 3-19, incorporated by reference herein). In one embodiment, nucleic acid molecules of the invention include one or more (e.g., about 1, 2, 3, 4, 5, 6, 7, 8, 9, 10, or more) G-clamp nucleotides, which are modified cytosine analogs which confer the ability to hydrogen bond both Watson-Crick and Hoogsteen faces of a complementary guanine within a duplex, and can provide for enhanced affinity and specificity to nucleic acid targets (see, e.g., Lin et al. 1998, J. Am. Chem. Soc., 120, 8531-8532, incorporated by reference herein). In another example, nucleic acid molecules can include one or more (e.g., about 1, 2, 3, 4, 5, 6, 7, 8, 9, 10, or more) LNA “locked nucleic acid” nucleotides such as a 2′,4′-C methylene bicyclo nucleotide (see, e.g., Wengel et al., WO 00/66604 and WO 99/14226).

siNA molecules can be provided as conjugates and/or complexes, e.g., to facilitate delivery of siNA molecules into a cell. Exemplary conjugates and/or complexes includes those composed of an siNA and a small molecule, lipid, cholesterol, phospholipid, nucleoside, antibody, toxin, negatively charged polymer (e.g., protein, peptide, hormone, carbohydrate, polyethylene glycol, or polyamine). In general, the transporters described are designed to be used either individually or as part of a multi-component system, with or without degradable linkers. These compounds can improve delivery and/or localization of nucleic acid molecules into cells in the presence or absence of serum (see, e.g., U.S. Pat. No. 5,854,038). Conjugates of the molecules described herein can be attached to biologically active molecules via linkers that are biodegradable, such as biodegradable nucleic acid linker molecules.

### Nucleic Acid Modifications

In some embodiments, a subject nucleic acid (e.g., a dsRNA, a siNA, etc.) has one or more modifications, e.g., a base modification, a backbone modification, etc., to provide the nucleic acid with a new or enhanced feature (e.g., improved stability). A nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound, however, linear compounds are suitable. In addition, linear compounds may have internal nucleotide base complementarity and may therefore fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

Suitable nucleic acid modifications include, but are not limited to: 2′Omethyl modified nucleotides, 2′ Fluoro modified nucleotides, locked nucleic acid (LNA) modified nucleotides, peptide nucleic acid (PNA) modified nucleotides, nucleotides with phosphorothioate linkages, and a 5′ cap (e.g., a 7-methylguanylate cap (m7G)). Additional details and additional modifications are described below.

A 2′-O-Methyl modified nucleotide (also referred to as 2′-O-Methyl RNA) is a naturally occurring modification of RNA found in tRNA and other small RNAs that arises as a post-transcriptional modification. Oligonucleotides can be directly synthesized that contain 2′-O-Methyl RNA. This modification increases Tm of RNA:RNA duplexes but results in only small changes in RNA:DNA stability. It is stabile with respect to attack by single-stranded ribonucleases and is typically 5 to 10-fold less susceptible to DNases than DNA. It is commonly used in antisense oligos as a means to increase stability and binding affinity to the target message.

2′ Fluoro modified nucleotides (e.g., 2′ Fluoro bases) have a fluorine modified ribose which increases binding affinity (Tm) and also confers some relative nuclease resistance when compared to native RNA. These modifications are commonly employed in ribozymes and siRNAs to improve stability in serum or other biological fluids.

LNA bases have a modification to the ribose backbone that locks the base in the C3′-endo position, which favors RNA A-type helix duplex geometry. This modification significantly increases Tm and is also very nuclease resistant. Multiple LNA insertions can be placed in an oligo at any position except the 3′-end. Applications have been described ranging from antisense oligos to hybridization probes to SNP detection and allele specific PCR. Due to the large increase in Tm conferred by LNAs, they also can cause an increase in primer dimer formation as well as self-hairpin formation. In some cases, the number of LNAs incorporated into a single oligo is 10 bases or less.

The phosphorothioate (PS) bond (i.e., a phosphorothioate linkage) substitutes a sulfur atom for a non-bridging oxygen in the phosphate backbone of a nucleic acid (e.g., an oligo). This modification renders the internucleotide linkage resistant to nuclease degradation. Phosphorothioate bonds can be introduced between the last 3-5 nucleotides at the 5′- or 3′-end of the oligo to inhibit exonuclease degradation. Including phosphorothioate bonds within the oligo (e.g., throughout the entire oligo) can help reduce attack by endonucleases as well.

In some embodiments, a subject nucleic acid (e.g., a dsRNA, a siNA, etc.) has one or more nucleotides that are 2′-O-Methyl modified nucleotides. In some embodiments, a subject nucleic acid (e.g., a dsRNA, a siNA, etc.) has one or more 2′ Fluoro modified nucleotides. In some embodiments, a subject nucleic acid (e.g., a dsRNA, a siNA, etc.) has one or more LNA bases. In some embodiments, a subject nucleic acid (e.g., a dsRNA, a siNA, etc.) has one or more nucleotides that are linked by a phosphorothioate bond (i.e., the subject nucleic acid has one or more phosphorothioate linkages). In some embodiments, a subject nucleic acid (e.g., a dsRNA, a siNA, etc.) has a 5′ cap (e.g., a 7-methylguanylate cap (m7G)). In some embodiments, a subject nucleic acid (e.g., a dsRNA, a siNA, etc.) has a combination of modified nucleotides. For example, a subject nucleic acid (e.g., a dsRNA, a siNA, etc.) can have a 5′ cap (e.g., a 7-methylguanylate cap (m7G)) in addition to having one or more nucleotides with other modifications (e.g., a 2′-O-Methyl nucleotide and/or a 2′ Fluoro modified nucleotide and/or a LNA base and/or a phosphorothioate linkage).

### Modified Backbones and Modified Internucleoside Linkages

Examples of suitable nucleic acids containing modifications include nucleic acids containing modified backbones or non-natural internucleoside linkages. Nucleic acids having modified backbones include those that retain a phosphorus atom in the backbone and those that do not have a phosphorus atom in the backbone.

Suitable modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Suitable oligonucleotides having inverted polarity comprise a single 3′ to 3′ linkage at the 3′-most internucleotide linkage i.e. a single inverted nucleoside residue which may be a basic (the nucleobase is missing or has a hydroxyl group in place thereof). Various salts (such as, for example, potassium or sodium), mixed salts and free acid forms are also included.

In some embodiments, a subject nucleic acid comprises one or more phosphorothioate and/or heteroatom internucleoside linkages, in particular —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—). MMI type internucleoside linkages are disclosed in the above referenced U.S. Pat. No. 5,489,677, the disclosure of which is incorporated herein by reference in its entirety. Suitable amide internucleoside linkages are disclosed in U.S. Pat. No. 5,602,240, the disclosure of which is incorporated herein by reference in its entirety.

Also suitable are nucleic acids having morpholino backbone structures as described in, e.g., U.S. Pat. No. 5,034,506. For example, in some embodiments, a subject nucleic acid comprises a 6-membered morpholino ring in place of a ribose ring. In some of these embodiments, a phosphorodiamidate or other non-phosphodiester internucleoside linkage replaces a phosphodiester linkage.

Suitable modified polynucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

### Mimetics

A subject nucleic acid can be a nucleic acid mimetic. The term “mimetic” as it is applied to polynucleotides is intended to include polynucleotides wherein only the furanose ring or both the furanose ring and the internucleotide linkage are replaced with non-furanose groups, replacement of only the furanose ring is also referred to in the art as being a sugar surrogate. The heterocyclic base moiety or a modified heterocyclic base moiety is maintained for hybridization with an appropriate target nucleic acid. One such nucleic acid, a polynucleotide mimetic that has been shown to have excellent hybridization properties, is referred to as a peptide nucleic acid (PNA). In PNA, the sugar-backbone of a polynucleotide is replaced with an amide containing backbone, in particular an aminoethylglycine backbone. The nucleotides are retained and are bound directly or indirectly to aza nitrogen atoms of the amide portion of the backbone.

One polynucleotide mimetic that has been reported to have excellent hybridization properties is a peptide nucleic acid (PNA). The backbone in PNA compounds is two or more linked aminoethylglycine units which gives PNA an amide containing backbone. The heterocyclic base moieties are bound directly or indirectly to aza nitrogen atoms of the amide portion of the backbone. Representative U.S. patents that describe the preparation of PNA compounds include, but are not limited to: U.S. Pat. Nos. 5,539,082; 5,714,331; and 5,719,262, the disclosures of which are incorporated herein by reference in their entirety.

Another class of polynucleotide mimetic that has been studied is based on linked morpholino units (morpholino nucleic acid) having heterocyclic bases attached to the morpholino ring. A number of linking groups have been reported that link the morpholino monomeric units in a morpholino nucleic acid. One class of linking groups has been selected to give a non-ionic oligomeric compound. The non-ionic morpholino-based oligomeric compounds are less likely to have undesired interactions with cellular proteins. Morpholino-based polynucleotides are non-ionic mimics of oligonucleotides which are less likely to form undesired interactions with cellular proteins (Dwaine A. Braasch and David R. Corey, Biochemistry, 2002, 41(14), 4503-4510). Morpholino-based polynucleotides are disclosed in U.S. Pat. No. 5,034,506, the disclosure of which is incorporated herein by reference in its entirety. A variety of compounds within the morpholino class of polynucleotides have been prepared, having a variety of different linking groups joining the monomeric subunits.

A further class of polynucleotide mimetic is referred to as cyclohexenyl nucleic acids (CeNA). The furanose ring normally present in a DNA/RNA molecule is replaced with a cyclohexenyl ring. CeNA DMT protected phosphoramidite monomers have been prepared and used for oligomeric compound synthesis following classical phosphoramidite chemistry. Fully modified CeNA oligomeric compounds and oligonucleotides having specific positions modified with CeNA have been prepared and studied (see Wang et al., J. Am. Chem. Soc., 2000, 122, 8595-8602, the disclosure of which is incorporated herein by reference in its entirety). In general the incorporation of CeNA monomers into a DNA chain increases its stability of a DNA/RNA hybrid. CeNA oligoadenylates formed complexes with RNA and DNA complements with similar stability to the native complexes. The study of incorporating CeNA structures into natural nucleic acid structures was shown by NMR and circular dichroism to proceed with easy conformational adaptation.

A further modification includes Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety. The linkage can be a methylene (—CH₂—), group bridging the 2′ oxygen atom and the 4′ carbon atom wherein n is 1 or 2 (Singh et al., Chem. Commun., 1998, 4, 455-456, the disclosure of which is incorporated herein by reference in its entirety). LNA and LNA analogs display very high duplex thermal stabilities with complementary DNA and RNA (Tm=+3 to +10° C.), stability towards 3′-exonucleolytic degradation and good solubility properties. Potent and nontoxic antisense oligonucleotides containing LNAs have been described (e.g., Wahlestedt et al., Proc. Natl. Acad. Sci. U.S.A., 2000, 97, 5633-5638, the disclosure of which is incorporated herein by reference in its entirety).

The synthesis and preparation of the LNA monomers adenine, cytosine, guanine, 5-methyl-cytosine, thymine and uracil, along with their oligomerization, and nucleic acid recognition properties have been described (e.g., Koshkin et al., Tetrahedron, 1998, 54, 3607-3630, the disclosure of which is incorporated herein by reference in its entirety). LNAs and preparation thereof are also described in WO 98/39352 and WO 99/14226, as well as U.S. applications 20120165514, 20100216983, 20090041809, 20060117410, 20040014959, 20020094555, and 20020086998, the disclosures of which are incorporated herein by reference in their entirety.

### Modified Sugar Moieties

A subject nucleic acid can also include one or more substituted sugar moieties. Suitable polynucleotides comprise a sugar substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C.sub.1 to C₁₀ alkyl or C₂ to C₁₀ alkenyl and alkynyl. Particularly suitable are O((CH₂)ₙO)ₘCH₃, O(CH₂)ₙOCH₃, O(CH₂)ₙNH₂, O(CH₂)ₙCH₃, O(CH₂)ₙONH₂, and O(CH₂)ₙON((CH₂)ₙCH₃)₂, where n and m are from 1 to about 10. Other suitable polynucleotides comprise a sugar substituent group selected from: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A suitable modification includes 2′-methoxyethoxy (2′-O—CH₂ CH₂OCH₃, also known as 2′-O-(2-methoxyethyl) or 2′-MOE) (Martin et al., Helv. Chim. Acta, 1995, 78, 486-504, the disclosure of which is incorporated herein by reference in its entirety) i.e., an alkoxyalkoxy group. A further suitable modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Other suitable sugar substituent groups include methoxy (—O—CH₃), aminopropoxy (—OCH₂CH₂CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. A suitable 2′-arabino modification is 2′-F. Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics such as cyclobutyl moieties in place of the pentofuranosyl sugar.

### Base Modifications and Substitutions

A subject nucleic acid may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (H-pyrido(3′,2′:4,5)pyrrolo(2,3-d)pyrimidin-2-one).

Heterocyclic base moieties may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993; the disclosures of which are incorporated herein by reference in their entirety. Certain of these nucleobases are useful for increasing the binding affinity of an oligomeric compound. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi et al., eds., Antisense Research and Applications, CRC Press, Boca Raton, 1993, pp. 276-278; the disclosure of which is incorporated herein by reference in its entirety) and are suitable base substitutions, e.g., when combined with 2′-O-methoxyethyl sugar modifications.

### Conjugates

Another possible modification of a subject nucleic acid involves chemically linking to the polynucleotide one or more moieties or conjugates which enhance the activity, cellular distribution or cellular uptake of the oligonucleotide. These moieties or conjugates can include conjugate groups covalently bound to functional groups such as primary or secondary hydroxyl groups. Conjugate groups include, but are not limited to, intercalators, reporter molecules, polyamines, polyamides, polyethylene glycols, polyethers, groups that enhance the pharmacodynamic properties of oligomers, and groups that enhance the pharmacokinetic properties of oligomers. Suitable conjugate groups include, but are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that enhance the pharmacokinetic properties include groups that improve uptake, distribution, metabolism or excretion of a subject nucleic acid.

Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N. Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937).

A conjugate may include a “Protein Transduction Domain” or PTD (also known as a CPP—cell penetrating peptide), which may refer to a polypeptide, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from extracellular space to intracellular space, or cytosol to within an organelle. In some embodiments, a PTD is covalently linked to the 3′ terminus of an exogenous polynucleotide (e.g., a dsRNA or siNA). In some embodiments, a PTD is covalently linked to the 5′ terminus of an exogenous polynucleotide (e.g., a dsRNA or siNA). Exemplary PTDs include but are not limited to a minimal undecapeptide protein transduction domain (corresponding to residues 47-57 of HIV-1 TAT comprising YGRKKRRQRRR (SEQ ID NO:1); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); an Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR (SEQ ID NO:2); Transportan GWTLNSAGYLLGKINLKALAALAKKIL (SEQIDNO:3);KALAWEAKLAKALAKALAKHLAKALA KALKCEA (SEQ ID NO:4); and RQIKIWFQNRRMKWKK(SEQ ID NO:5). Exemplary PTDs include but are not limited to, YGRKKRRQRRR (SEQ ID NO:1), RKKRRQRRR (SEQ ID NO:6); an arginine homopolymer of from 3 arginine residues to 50 arginine residues; Exemplary PTD domain amino acid sequences include, but are not limited to, any of the following: YGRKKRRQRRR (SEQ ID NO: 1); RKKRRQRR (SEQ ID NO:7); YARAAARQARA (SEQ ID NO:8); THRLPRRRRRR (SEQ ID NO:9); and GGRRARRRRRR (SEQ ID NO: 10). In some embodiments, the PTD is an activatable CPP (ACPP) (Aguilera et al. (2009) Integr Biol (Camb) June; 1(5-6): 371-381). ACPPs comprise a polycationic CPP (e.g., Arg9 or “R9”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane.

The application of RNAi technology (e.g., an siRNA molecule) in a method of the present disclosure can occur in several ways, each resulting in functional silencing of a gene product in an adult stem cell. Functional gene silencing by an RNAi agent (e.g., an siRNA molecule) does not necessarily include complete inhibition of the targeted gene product. In some cases, marginal decreases in gene product expression caused by an RNAi agent can translate to significant functional or phenotypic changes in the host cell, tissue, organ, or animal. Therefore, gene silencing is understood to be a functional equivalent and the degree of gene product degradation to achieve silencing may differ between gene targets or host cell type. Gene silencing may decrease gene product expression by 1%, 2%, 3%, 4%, 5%, 6%, 7%, 8%, 9%, or 10%. Preferentially, gene product expression is decreased by 10%, 20%, 30%, 40%, 50%, 60%, 70%, 80%, 90%, or 100% (i.e., complete inhibition).

An interfering nucleic acid can be introduced into an adult stem cell in any of a variety of ways. In some embodiments, a recombinant adenoviral vector is used. Recombinant adenoviral vectors offer several significant advantages for the expression nucleic acids (e.g., an siRNA) in stem cells. The viruses can be prepared at extremely high titer, infect non-replicating cells, and confer high-efficiency and high-level transduction of target cells in vivo after directed injection or perfusion. Furthermore, as adenoviruses do not integrate their DNA into the host genome, there is a reduced risk of inducing spontaneous proliferative disorders. In animal models, adenoviral gene transfer has generally been found to mediate high-level expression for approximately one week. The duration of transgene expression may be prolonged, and ectopic expression reduced, by using tissue-specific promoters. Other improvements in the molecular engineering of the adenoviral vector itself have produced more sustained transgene expression and less inflammation. This is seen with so-called “second generation” vectors harboring specific mutations in additional early adenoviral genes and “gutless” vectors in which virtually all the viral genes are deleted utilizing a cre-lox strategy (Engelhardt et al., Proc. Natl. Acad. Sci. USA 91:6196-6200 (1994) and Kochanek et al., Proc. Natl. Acad. Sci. USA 93:5731-5736 (1996)). In addition, recombinant adeno-associated viruses (rAAV), derived from non-pathogenic parvoviruses, can be used to express a polypeptide or oligonucleotide, as these vectors elicit almost no cellular immune response, and produce transgene expression lasting months in most systems. Incorporation of a tissue-specific promoter may also be beneficial.

Other viral vectors useful for the delivery of an interfering nucleic acid into an adult stem cell are retroviruses, including lentiviruses. As opposed to adenoviruses, the genetic material in retroviruses is RNA, while the genetic material of their hosts is in the form of DNA. When a retrovirus infects a host cell, it introduces its RNA together with enzymes into the cell. This RNA molecule is used to produce a double-stranded DNA copy (provirus) by reverse transcription. Following transport into the cell nucleus, the proviral DNA is integrated in a host chromosome, permanently altering the genome of the infected cell and any progeny cells that may arise. Retroviruses include lentiviruses, a family of viruses including human immunodeficiency virus (HIV) that includes several accessory proteins to facilitate viral infection and proviral integration.

Other transfections approaches, including naked DNA or oligonucleotides (e.g., DNA vectors such as plasmids) encoding an RNA interference molecule (e.g., an siRNA or shRNA), can be used to genetically modify stem cells. Improved transfection efficiency of naked DNA can be achieved using electroporation or a “gene gun,” which shoots DNA-coated gold particles into the cell using high pressure gas.

To improve the delivery of a DNA vector (e.g., a plasmid) into a stem cell, the DNA can be protected from damage and its entry into the cell facilitated, for example, by using lipoplexes and polyplexes. Plasmid DNA can be covered with lipids in an organized structure like a micelle or a liposome. When the organized structure is complexed with DNA it is called a lipoplex. There are three types of lipids, anionic (negatively-charged), neutral, or cationic (positively-charged). Lipoplexes that utilize cationic lipids have proven utility for gene transfer. Cationic lipids, due to their positive charge, naturally complex with the negatively charged DNA. Also as a result of their charge, they interact with the cell membrane. Endocytosis of the lipoplex then occurs, and the DNA is released into the cytoplasm. The cationic lipids also protect against degradation of the DNA by the cell.

Complexes of polymers with DNA are called polyplexes. Most polyplexes consist of cationic polymers and their production is regulated by ionic interactions. One large difference between the methods of action of polyplexes and lipoplexes is that polyplexes cannot release their DNA load into the cytoplasm, so to this end, co-transfection with endosome-lytic agents (to lyse the endosome that is made during endocytosis) such as inactivated adenovirus must occur. However, this is not always the case; polymers such as polyethylenimine have their own method of endosome disruption as does chitosan and trimethylchitosan.

Dendrimers, a highly branched macromolecule with a spherical shape, may be also be used to genetically modify stem cells of the invention. The surface of the dendrimer particle may be functionalized to alter its properties. In particular, it is possible to construct a cationic dendrimer (i.e., one with a positive surface charge). When in the presence of genetic material such as a DNA plasmid, charge complementarity leads to a temporary association of the nucleic acid with the cationic dendrimer. On reaching its destination, the dendrimer-nucleic acid complex can be taken into a stem cell of the invention by endocytosis.

### Caspase-1 Inhibitors

As noted above, in some cases, a method of the present disclosure comprises contacting an adult stem cell with a caspase-1 inhibitor. In some cases, the caspase-1 inhibitor is a compound as described in U.S. Pat. No. 9,245,290. In some cases, the caspase-1 inhibitor is z-VAD-fmk; see, e.g., Lipinska et al. (2014) J. Immunol. Methods 411:66. In some cases, the caspase-1 inhibitor is ac-YVAD-cmk; see, e.g., Lipinska et al. (2014) J. Immunol. Methods 411:66. In some cases, the caspase-1 inhibitor is VX-765. In some cases, the caspase-1 inhibitor is z-WEHD-fmk. See, e.g., Fischer and Schulze-Osthoff (2005) Cell Death and Differentiation 12:942.

VX-765 is (S)-1-((S)-2-{[1-(4-Amino-3-chloro-phenyl)-methanoyl]-amino-3,3-dimethyl-butanoyl)-pyrrolidine-2-carboxylic acid ((2R,3S)-2-ethoxy-5-oxo-tetrahydro-furan-3-yl)-amide. See, e.g., Wannamaker et al. (2007) J. Pharmacol. Exp. Ther. 321:509.

The caspase-1 inhibitor ac-YVAD-cmk has the following structure:

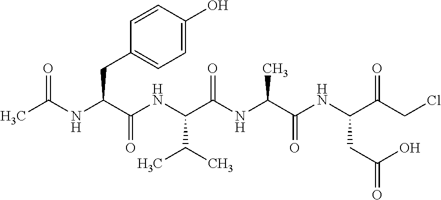 [SMILES: CC(=O)N[C@@H](Cc1ccc(O)cc1)C(=O)N[C@H](C(=O)N[C@@H](C)C(=O)N[C@@H](CC(=O)O)C(=O)CCl)C(C)C]

The caspase-1 inhibitor z-VAD-fmk is N-benzyloxycarbonyl-Val-Ala-Asp(O-Me)-fluoromethyl ketone.

The caspase-1 inhibitor z-WEHD-fmk is N-benzyloxycarbonyl-Trp-Glu-His-Asp-fluoromethyl ketone.

Improving the Function of Stem Cells, and/or Inhibiting or Reversing Stem Cell Aging in an Individual

The present disclosure provides a method of improving the function of stem cells, and/or inhibiting or reversing aging of an adult stem cell in an individual, the method comprising administering to the individual an agent that: a) increases the level of a SIRT2 polypeptide in the adult stem cell; b) increases the deacetylase activity of a SIRT2 polypeptide in the adult stem cell; c) increases deacetylation of a nucleotide-binding domain and leucine-rich repeat-containing-3 (NLRP3) polypeptide in the adult stem cell; d) reduces the level and/or activity of an NLRP3 polypeptide in the adult stem cell; e) reduces the level and/or activity of a caspase-1 polypeptide in the adult stem cell; or f) inhibits pyroptosis in the adult stem cell.

The present disclosure provides a method of improving the function of stem cells, and/or inhibiting or reversing aging of an adult stem cell in an individual, the method comprising: i) carrying out, in vitro, one or more of: a) increasing the level of a SIRT2 polypeptide; b) increasing the deacetylase activity of a SIRT2 polypeptide; c) increasing deacetylation of a nucleotide-binding domain and leucine-rich repeat-containing-3 (NLRP3) polypeptide; d) reducing the level and/or activity of an NLRP3 polypeptide; e) reducing the level and/or activity of a caspase-1 polypeptide; and f) inhibiting pyroptosis; in an adult stem cell obtained from the individual, thereby modifying the adult stem cell; and ii) introducing the modified adult stem cell into the individual.

Because Nicotinamide Adenine Dinucleotide (NAD⁺) is required for the activity of all SIRT proteins, and NAD⁺ levels decline during aging, raising NAD⁺ levels would antagonize vascular aging. P2X7R is one example of a receptor that transports NADH across the plasma membrane of astrocytes in the brain and spinal cord, resulting in an increase in intracellular NADH and NAD⁺ levels. Once an NADH molecule has been transported into the cytoplasm of a cell, it is generally oxidized directly into NAD⁺ by dehydrogenase enzymes such as Lactate Dehydrogenase (LDH), Glyceraldehyde 3-Phosphate Dehydrogenase (G3PDH), or Malate dehydrogenase (MDH)/the malate-aspartate shuttle. NADH may also be broken down or catabolized into NAD precursors, first converting to NAD⁺, then to NMN and NR respectively, before recycling back to NAD. Supplementing NADH and CoQ10 has been reported to increase NAD⁺ levels. NAD⁺ levels can also be increased either by giving NAD⁺ precursors, by inhibiting NAD⁺ consumption, or by enhancing NAMPT-mediated NAD⁺ salvage from nicotinamide (NAM).

In some cases, an agent suitable for use in a method of the present disclosure is an agent that increases the activity of SIRT2. In some cases, a SIRT2 activator suitable for use in a method of the present disclosure also activates SIRT1. In some cases, a SIRT2 activator suitable for use in a method of the present disclosure is specific for SIRT2, i.e., the SIRT2 activator increases the activity of SIRT2 but does not substantially increase the activity of SIRT1.

In some cases, an agent suitable for use in a method of the present disclosure is a SIRT2 activator. Suitable SIRT2 activators include, but are not limited to, certain 1,4-dihydropyridine (DHP) derivatives bearing a benzyl group at the N1 position (Mai, et al., (2009) J. Med. Chem. 52:5496-5504); SRT1720 HCl (Milne, et al., (2007) Nature 450(7170):712-716; Villalba and Alcain (2012) Biofactors 38(5):349-359); and Sirtuin-activating compounds (STACs), such as Fisetin (7,3′,4′-flavon-3-ol) (Guedes-Dias and Oliveira, Biochimica et Biophysica Acta, 1832 (2013) 1345-1359).

SRT1720 has the following structure:

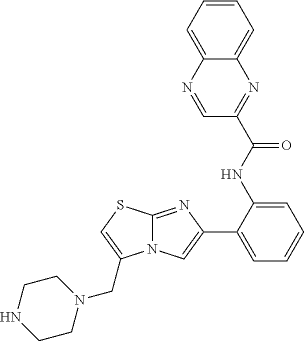 [SMILES: O=C(Nc1ccccc1-c1cn2c(CN3CCNCC3)csc2n1)c1cnc2ccccc2n1]

Because P2X7R is a receptor that transports NADH across the plasma membrane of astrocytes, and intracellular NADH gets oxidized into NAD⁺ by LDH, G3PDH, or MDH, any agent that increases the levels or activity of P2X7R, LDH, G3PDH and/or MDH would be expected to increase intracellular NAD⁺ levels. Thus, in some cases, an agent suitable for use in a method of the present disclosure is an agent that increases intracellular NAD⁺ levels. In some cases, an agent suitable for use in a method of the present disclosure is an agent that increases the levels or activity of P2X7R. In some cases, NAD⁺ levels are increased by small molecules that activate NAD⁺ synthesis enzymes. In some cases, NAD⁺ levels are increased by increasing the level or activity of an NAD⁺ biosynthesis enzyme in the stem cell (e.g., LDH, G3PDH, MDH). In some cases, NAD⁺ levels are increased by increasing the level or activity of an NAD⁺ precursor, such as NMN or NR, in the stem cell. In some cases, the agent that increases intracellular NAD⁺ levels results in activation of SIRT2.

In some cases, an agent suitable for use in a method of the present disclosure is an NLRP3 inhibitor. Suitable inhibitors of NLRP3 include, but are not limited to, glyburide and 16673-34-0 (5-Chloro-2-methoxy-N-[2-(4-sulfamoylphenyl)ethyl]benzamide) (Marchetti, et al., J. Cardioovasc. Pharmacol. 2014, 63(4):316-322); MCC950 (a.k.a. CP-456773; CRID3; N-((1,2,3,5,6,7-hexahydro-s-indacen-4-yl)carbamoyl)-4-(2-hydroxypropan-2-yl)furan-2-sulfonamide) (Coll, R. C., et al., 2015, Nat. Med. 21: 248); Shikonin (Zorman, et al, 2016, PLoS ONE 11(7):e0159826); sodium butyrate (NaB); and β-hydroxybutyrate (“BHB” or “HBA”) (Bian, et al., Int'l. J. Mol. Sciences. 2017 18(3):562; Youm, et al., Nat. Med. 2015, 21(3):263-269). In some cases, the NLRP3 inhibitor is a small molecule. In some cases, the NLRP3 inhibitor is an inhibitory RNA molecule, e.g., an siRNA that specifically reduces the level or activity of NLRP3.

Shikonin has the following structure:

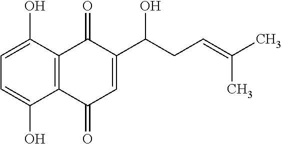 [SMILES: CC(C)=CCC(O)C1=CC(=O)c2c(O)ccc(O)c2C1=O]

A method of the present disclosure for improving the function of stem cells, and/or inhibiting or reversing aging of an adult stem cell in an individual can reduce tissue degeneration or injury in the individual. Thus, the present disclosure provides a method of reducing tissue degeneration in an individual, the method comprising improving the function of stem cells, and/or inhibiting or reversing aging of an adult stem cell in the individual.

A method of the present disclosure for improving the function of stem cells, and/or inhibiting or reversing aging of an adult stem cell in an individual can treat or ameliorate a tissue degenerative disease in the individual. Examples of tissue degenerative diseases include neurodegenerative diseases (e.g., Alzheimer's disease), muscle degenerative diseases (e.g., muscular dystrophy), and bone marrow failure. Thus, the present disclosure provides a method of treating or ameliorating a tissue degenerative disease, including neurodegenerative diseases (e.g., Alzheimer's Disease), muscle degenerative diseases (e.g., muscular dystrophy), and bone marrow failure, in an individual, the method comprising improving the function of stem cells, and/or inhibiting or reversing aging of an adult stem cell in the individual.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-23 are provided below.

As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A method for improving the function of stem cells, and/or inhibiting or reversing aging of an adult stem cell, the method comprising one or more of: a) increasing the level and/or activity of a SIRT2 polypeptide in the adult stem cell; b) increasing the deacetylase activity of a SIRT2 polypeptide in the adult stem cell; c) increasing deacetylation of a nucleotide-binding domain and leucine-rich repeat-containing-3 (NLRP3) polypeptide in the adult stem cell; d) reducing the level and/or activity of an NLRP3 polypeptide in the adult stem cell; e) reducing the level and/or activity of a caspase-1 polypeptide in the adult stem cell; and f) inhibiting pyroptosis in the adult stem cell.

Aspect 2. The method of aspect 1, wherein the adult stem cell is a muscle stem, a hematopoietic stem cell, an epithelial stem cell, a neural stem cell, a mesenchymal stem cell, a mammary stem cell, an intestinal stem cell, a mesodermal stem cell, an endothelial stem cell, an olfactory stem cell, or a neural crest stem cell.

Aspect 3. The method of aspect 1, wherein the adult stem cell is a rodent adult stem cell, a human adult stem cell, or a non-human primate adult stem cell.

Aspect 4. The method of aspect 1, wherein reduction of the level of an NLRP3 polypeptide in the adult stem cell comprises introduction into the adult stem cell of an inhibitory nucleic acid that specifically reduces the level of NLRP3 mRNA and/or NLRP3 polypeptide in the adult stem cell.

Aspect 5. The method of aspect 1, wherein the a) increase in the level and/or activity of the SIRT2 polypeptide, b) increase in the deacetylase activity of the SIRT2 polypeptide, c) increase in deacetylation of the NLRP3, d) reduction in the level and/or activity of the NLRP3 polypeptide, or e) reduction in the level and/or activity of the caspase-1 polypeptide in the adult stem cell is achieved by administration of: (i) an activator of a SIRT selected from: a 1,4-dihydropyridine (DHP) derivative bearing a benzyl group at the N1 position, SRT1720 HCl, and Fisetin; or (ii) an inhibitor of NLRP3 selected from: glyburide, 16673-34-0, MCC950, Shikonin, sodium butyrate, (3-hydroxybutyrate, and an siRNA targeting NLRP3 mRNA; or (iii) an inhibitor of caspase-1 selected from: z-VAD-fmk, ac-YVAD-cmk, VX-765, and z-WEHD-fmk; or (iv) an agent that increases intracellular NAD⁺ levels selected from agents that increases levels or activity of: a P2X7R receptor, extracellular NADH, CoQ10, LDH, G3PDH, MDH, NMN and NR.

Aspect 6. The method of aspect 4, wherein the inhibitory nucleic acid is a short interfering nucleic acid, a short hairpin RNA, a ribozyme, or an antisense nucleic acid.

Aspect 7. The method of aspect 6, wherein the inhibitory nucleic acid comprises one or more of a base modification, a backbone modification, a modified internucleoside linkage, and a modified sugar moiety.

Aspect 8. The method of aspect 1, wherein reduction of the level of the caspase-1 polypeptide in the adult stem cell comprises introduction into the adult stem cell of an inhibitory nucleic acid that specifically reduces the level of caspase-1 mRNA and/or caspase-1 polypeptide in the adult stem cell.

Aspect 9. The method of aspect 8, wherein the inhibitory nucleic acid is a short interfering nucleic acid, a short hairpin RNA, a ribozyme, or an antisense nucleic acid.

Aspect 10. The method of aspect 9, wherein the inhibitory nucleic acid comprises one or more of a base modification, a backbone modification, a modified internucleoside linkage, and a modified sugar moiety.

Aspect 11. The method of aspect 1, wherein reduction of the activity of the caspase-1 polypeptide in the adult stem cell comprises contacting the stem cell with a compound that inhibits caspase-1 activity.

Aspect 12. The method of aspect 1, wherein increasing the level of a SIRT2 polypeptide in the adult stem cell comprises introducing into the adult stem cell a nucleic acid comprising a nucleotide sequence encoding a SIRT2 polypeptide.

Aspect 13. The method of aspect 12, wherein the nucleotide sequence is operably linked to a promoter.

Aspect 14. The method of aspect 13, wherein the promoter is inducible.

Aspect 15. The method of aspect 12, wherein the nucleic acid is a recombinant expression vector.

Aspect 16. The method of aspect 15, wherein the expression vector is a recombinant viral vector.

Aspect 17. A method of improving the function of stem cells, and/or inhibiting or reversing aging of an adult stem cell in an individual, the method comprising administering to the individual an agent that: a) increases the level of a SIRT2 polypeptide in the adult stem cell; b) increases the deacetylase activity of a SIRT2 polypeptide in the adult stem cell; c) increases deacetylation of a nucleotide-binding domain and leucine-rich repeat-containing-3 (NLRP3) polypeptide in the adult stem cell; d) reduces the level and/or activity of an NLRP3 polypeptide in the adult stem cell; e) reduces the level and/or activity of a caspase-1 polypeptide in the adult stem cell; or f) inhibits pyroptosis in the adult stem cell.

Aspect 18: The method of aspect 17, wherein the agent is: (i) an activator of a SIRT selected from: a 1,4-dihydropyridine (DHP) derivative bearing a benzyl group at the N1 position, SRT1720 HCl, and Fisetin; or (ii) an inhibitor of NLRP3 selected from: glyburide, 16673-34-0, MCC950, Shikonin, sodium butyrate, β-hydroxybutyrate, and an siRNA targeting NLRP3 mRNA; or (iii) an inhibitor of caspase-1 selected from: z-VAD-fmk, ac-YVAD-cmk, VX-765, and z-WEHD-fmk; or (iv) an agent that increases intracellular NAD⁺ levels selected from agents that increase the levels or activity of: a P2X7R receptor, extracellular NADH, CoQ10, LDH, G3PDH, MDH, NMN and NR.

Aspect 19. A method of improving the function of stem cells, and/or inhibiting or reversing aging of an adult stem cell in an individual, the method comprising: i) carrying out, in vitro, one or more of: a) increasing the level of a SIRT2 polypeptide; b) increasing the deacetylase activity of a SIRT2 polypeptide; c) increasing deacetylation of a nucleotide-binding domain and leucine-rich repeat-containing-3 (NLRP3) polypeptide; d) reducing the level and/or activity of an NLRP3 polypeptide; e) reducing the level and/or activity of a caspase-1 polypeptide; and f) inhibiting pyroptosis; in an adult stem cell obtained from the individual, thereby modifying the adult stem cell; and ii) introducing the modified adult stem cell into the individual.

Aspect 20: The method of aspect 19, wherein the a) increase in the level and/or activity of the SIRT2 polypeptide, b) increase in the deacetylase activity of the SIRT2 polypeptide, c) increase in deacetylation of the NLRP3, or d) reduction in the level and/or activity of the NLRP3 polypeptide in the adult stem cell is achieved by administration of: (i) an activator of a SIRT selected from: a 1,4-dihydropyridine (DHP) derivative bearing a benzyl group at the N1 position, SRT1720 HCl, and Fisetin; or (ii) an inhibitor of NLRP3 selected from: glyburide, 16673-34-0, MCC950, Shikonin, sodium butyrate, β-hydroxybutyrate, and an siRNA targeting NLRP3 mRNA; or (iii) an inhibitor of caspase-1 selected from: z-VAD-fmk, ac-YVAD-cmk, VX-765, and z-WEHD-fmk; or (iv) an agent that increases intracellular NAD⁺ levels selected from agents that increase the levels or activity of: a P2X7R receptor, extracellular NADH, CoQ10, LDH, G3PDH, MDH, NMN and NR.

Aspect 21: A method of reducing tissue degeneration in an individual, the method comprising administering to the individual an agent that: a) increases the level of a SIRT2 polypeptide in the adult stem cell; b) increases the deacetylase activity of a SIRT2 polypeptide in the adult stem cell; c) increases deacetylation of a nucleotide-binding domain and leucine-rich repeat-containing-3 (NLRP3) polypeptide in the adult stem cell; d) reduces the level and/or activity of an NLRP3 polypeptide in the adult stem cell; e) reduces the level and/or activity of a caspase-1 polypeptide in the adult stem cell; or f) inhibits pyroptosis in the adult stem cell.

Aspect 22: A method of treating or ameliorating a tissue degenerative disease, including neurodegenerative diseases (e.g., Alzheimer's Disease), muscle degenerative diseases (e.g., muscular dystrophy), and bone marrow failure, in an individual, the method comprising administering to the individual an agent that: a) increases the level of a SIRT2 polypeptide in the adult stem cell; b) increases the deacetylase activity of a SIRT2 polypeptide in the adult stem cell; c) increases deacetylation of a nucleotide-binding domain and leucine-rich repeat-containing-3 (NLRP3) polypeptide in the adult stem cell; d) reduces the level and/or activity of an NLRP3 polypeptide in the adult stem cell; e) reduces the level and/or activity of a caspase-1 polypeptide in the adult stem cell; or f) inhibits pyroptosis in the adult stem cell.

Aspect 23: The method of aspect 21 or aspect 22, wherein the agent is: (i) an activator of a SIRT selected from: a 1,4-dihydropyridine (DHP) derivative bearing a benzyl group at the N1 position, SRT1720 HCl, and Fisetin; or (ii) an inhibitor of NLRP3 selected from: glyburide, 16673-34-0, MCC950, Shikonin, sodium butyrate, β-hydroxybutyrate, and an siRNA targeting NLRP3 mRNA; or (iii) an inhibitor of caspase-1 selected from: z-VAD-fmk, ac-YVAD-cmk, VX-765, and z-WEHD-fmk; or (iv) an agent that increases intracellular NAD⁺ levels selected from agents that increase the levels or activity of: a P2X7R receptor, extracellular NADH, CoQ10, LDH, G3PDH, MDH, NMN and NR.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Materials and Methods

### Cell Culture and RNAi

Double-stranded siRNAs were purchased from Qiagen and were transfected into cells via RNAiMax (Invitrogen) according to manufacturer's instructions. Mouse SIRT2 siRNA targeting sequence is 5′-CCAGAATAAGGCATTTCTCTA-3′ (SEQ ID NO:45). Mouse SIRT1 siRNA targeting sequence is AAGCGGCTTGAGGGTAATCAA (SEQ ID NO:46). The control for siRNA is non-targeting control (Qiagen). To reconstitute NLRP3 KO macrophages with wild-type (WT) or NLRP3 mutants, WT or NLRP3 mutants were cloned into pMSCVgfp retroviral construct. Retrovirus was generated by transfecting 293T cells (ATCC) with pMSCVgfp retroviral constructs as well as VSV-G and gag/pol expression vectors using Lipofectamine 2000 transfection kit (Invitrogen). 48 hours posttransfection, filtered retrovirus-enriched culture supernatant supplemented with 10 μg/ml of polybrene was applied to NLRP3 KO macrophages (a gift from E. Alnemri). The cells were subjected to another cycle of infection on the next day. To induce caspase-1 activation, NLRP3 KO macrophages reconstituted with WT or NLRP3 mutants were primed with 500 ng/ml LPS for 5 hours and then stimulated with 6 mM ATP for 1 hour. Proteins from cell media were trichloroacetic acid (TCA) precipitated for Western analyses.

### Pyroptosome Formation

Pyroptosome formation was performed as previously described⁴⁶. Briefly, 293T cells were co-transfected with ASC-EGFP and WT or mutant NLRP3 or control vector. 48 hours posttransfection, cells were observed using fluorescence microscopy.

### Immunoprecipitations

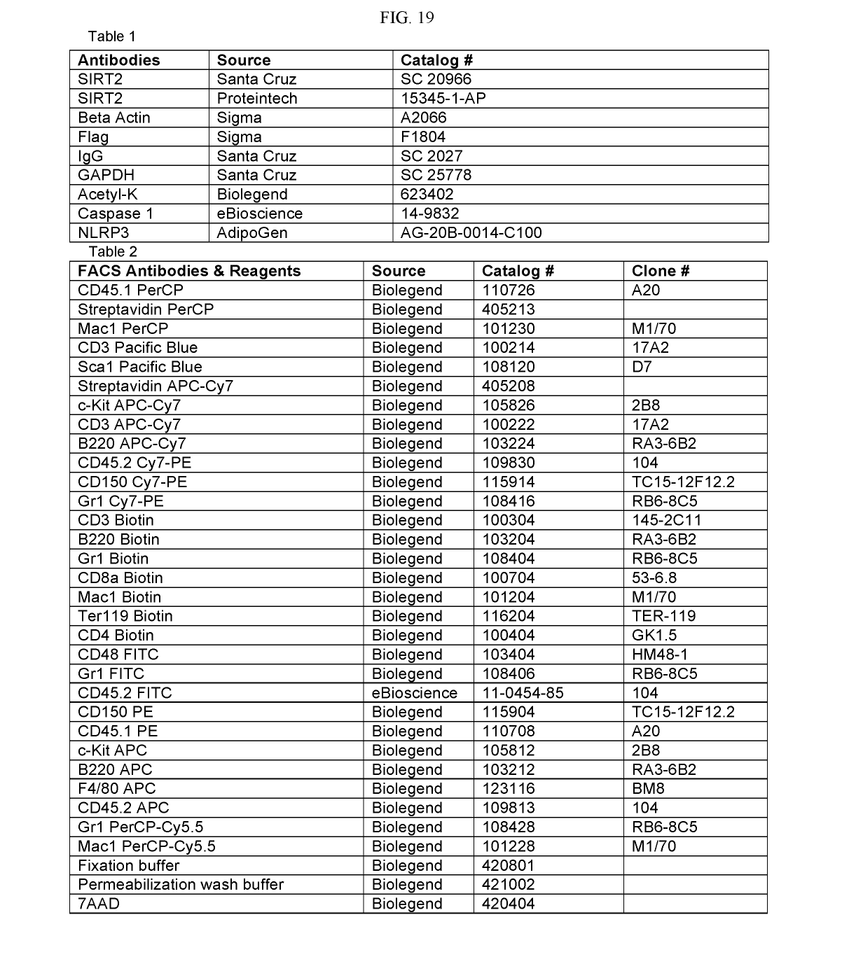

Immunoprecipitations were performed as previously described⁴⁷ with Flag-resin (Sigma). Elution was performed with either Flag peptide (Sigma) for western analyses or 100 mM Glycine solution (pH 3) for mass spectrometry analyses. Antibodies and reagents are provided in FIG. 19 (Table 1 and Table 2).

### Immunocytochemistry

Immunocytochemistry of HSCs was performed as previously described⁴⁸. Briefly, cells were directly sorted onto a glass slide, fixed with 4% paraformaldehyde (PFA), and stained with SIRT2 antibody. Nuclei were identified by staining with DAPI. Subcellular localizations were determined using confocal microscopy.

mRNA Analysis

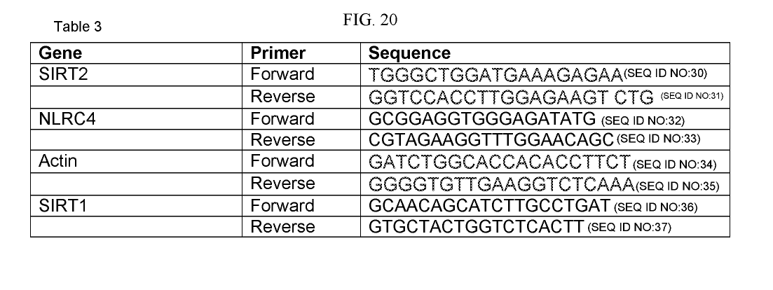

RNA was isolated from cells using Trizol reagent (Invitrogen). cDNA was generated using the qScript™ cDNA SuperMix (Quanta Biosciences). Gene expression was determined by real time PCR using Eva qPCR SuperMix kit (BioChain Institute) on an ABI StepOnePlus system. All data were normalized to β-Actin expression. PCR primer details are provided in FIG. 20 (Table 3).

### Mice

SIRT2 knockout mice and caspase 1 knockout mice have been described previously^{23,49}. All mice were housed on a 12:12 hr light:dark cycle at 25° C. All animal procedures were performed using age- and gender-matched mice in accordance with the animal care committee.

### Flow Cytometry and Cell Sorting.

Bone marrow cells were obtained by crushing the long bones with sterile PBS without calcium and magnesium supplemented with 2% FBS. Lineage staining contained a cocktail of biotinylated anti-mouse antibodies to Mac-1 (CD11b), Gr-1 (Ly-6G/C), Ter119 (Ly-76), CD3, CD4, CD8a (Ly-2), and B220 (CD45R) (BioLegend). For detection or sorting, streptavidin conjugated to APC-Cy7, c-Kit-APC, Sca-1-Pacific blue, CD48-FITC, and CD150-PE (BioLegend) was used. For congenic strain discrimination, anti-CD45.1 PerCP and anti-CD45.2 PE-Cy7 antibodies (BioLegend) were used. For assessment of cell viability, 7AAD staining (BioLegend) was performed according to the manufacturer's recommendation after cell surface staining. To determine intracellular activation of specific caspases, fluorescent labelled inhibitors of caspases (FLICA) probe assays (ImmunoChemistry Technologies) and active caspase 3 detection kits (BD Pharmingen) were used based on the manufacturer's instructions. All data were collected on a Fortessa (Becton Dickinson) and data analysis was performed with FlowJo (TreeStar). For cell sorting, lineage depletion or c-kit enrichment was performed according to the manufacturer's instructions (Miltenyi Biotec). Cells were sorted using a Cytopeia INFLUX Sorter (Becton Dickinson). Antibody details are provided in FIG. 19 (Table 1 and Table 2).

### Lentiviral Transduction of HSCs

As previously described⁵⁰, sorted HSCs were prestimulated for 5-10 hr in a 96 well U bottom dish in StemSpan SFEM (Stem Cell Technologies) supplemented with 10% FBS (Stem Cell Technologies), 1% Penicillin/Streptomycin (Invitrogen), IL3 (20 ng/rml), IL6 (20 ng/ml), TPO (50 ng/ml), Flt3L (50 ng/ml), and SCF (100 ng/ml) (Peprotech).

SIRT2 was cloned into the pFUGw lentiviral construct. shRNAs were cloned into pFUGw-H1 lentiviral construct. For cloning of shRNA vectors, the following oligonucleotides were used.

Caspase 1 targeting sequence 1
sense:
(SEQ ID NO: 47)
5′-ATTCCACGTCTTGCCCTCATTATTCAAGAGATAATGAGGGCAAGACGT
GTTTTTTTG-3′;
antisense:
(SEQ ID NO: 48)
5′-GATCCAAAAAAACACGTCTTGCCCTCATTATCTCTTGAATAATGAGGG
CAAGACGTGG-3′;
Caspase 1 targeting sequence 2
sense:
(SEQ ID NO: 49)
5′-ATTCGGACAATAAATGGATTGTTGGTCAAGAGCCAACAATCCATTTAT
TGTCCG-3′;
antisense:
(SEQ ID NO: 50)
5′-GATCCGGACAATAAATGGATTGTTGGCTCTTGACCAACAATCCATTTA
TTGTCCG-3′;
NLRP3
sense:
(SEQ ID NO: 51)
5′-ATTCCCACATGACTTTCCAGGAGTTTCAAGAGAACTCCTGGAAAGTCA
TGTGGTTTTTTG-3′;
antisense:
(SEQ ID NO: 52)
5′-GATCCAAAAAACCACATGACTTTCCAGGAGTTCTCTTGAAACTCCTGG
AAAGTCATGTGGG-3′;
NLRC4
sense:
(SEQ ID NO: 53)
5′-ATTCGGGTGAAGATATCGACATAATTCAAGAGATTATGTCGATATCTT
CACCCTTTTTTG-3′;
antisense:
(SEQ ID NO: 54)
5′-GATCCAAAAAAGGGTGAAGATATCGACATAATCTCTTGAATTATGTCG
ATATCTTCACCCG-3′.

Lentivirus was produced as described⁴¹, concentrated by centrifugation, and resuspended with supplemented StemSpan SFEM media. The lentiviral media were added to HSCs in a 96 well plate, spinoculated for 90 min at 270G in the presence of 8 ug/ml polybrene. This process was repeated 24 hr later with a fresh batch of lentiviral media.

### Transplantation Assays

250 sorted HSCs were mixed with 5×10⁵ CD45.1 B6.SJL competitor cells and injected into lethally irradiated B6.SJL recipient mice. To assess multilineage reconstitution of transplanted mice, peripheral blood was collected every month for 4 months by retroorbital bleeding. Red blood cells were lysed and the remaining blood cells were stained with CD45.2 FITC, CD45.1 PE, MacI PerCP, Gr1 Cy7PE, B220 APC, and CD3 PB (Biolegend). Bone marrow cells were analyzed 4 months posttranplantation. Antibody details are provided in FIG. 19 (Table 1 and Table 2).

### Statistical Analysis

No statistical methods were used to predetermine sample size. The number of mice chosen for each experiment is comparable to published literature for the same assays performed. Mice were randomized to groups and analysis of mice and tissue samples was performed by investigators blinded to the treatment or the genetic background of the animals. No data were excluded. Statistical analysis was performed with Excel (Microsoft). Means between two groups were compared with two-tailed, unpaired Student's t-test. Interaction between the variables was analyzed by 2-way ANOVA. Error Bars represent standard errors. In all corresponding figures, * represents p<0.05. ** represents p<0.01. *** represents p<0.001. ns represents p>0.05.

### Results

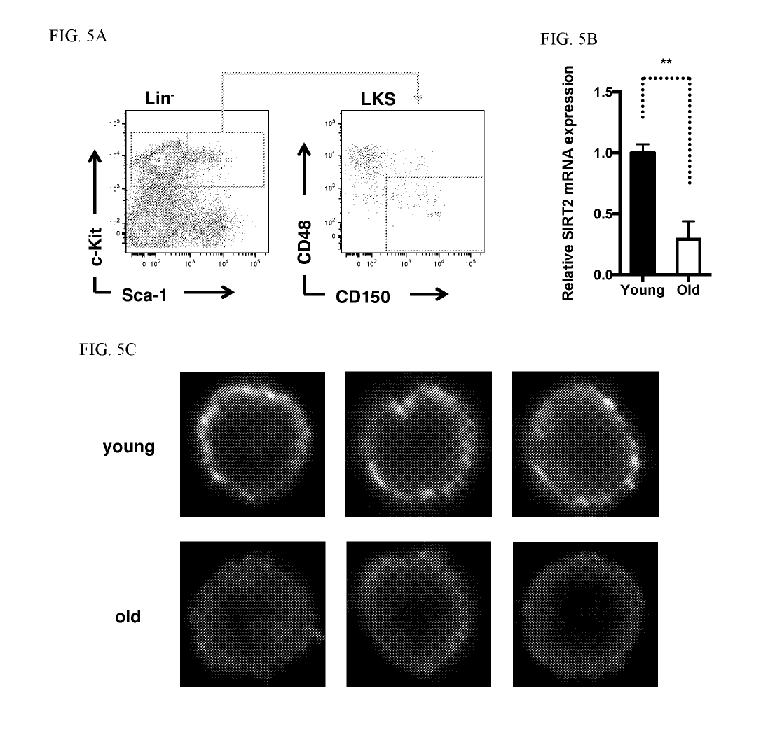

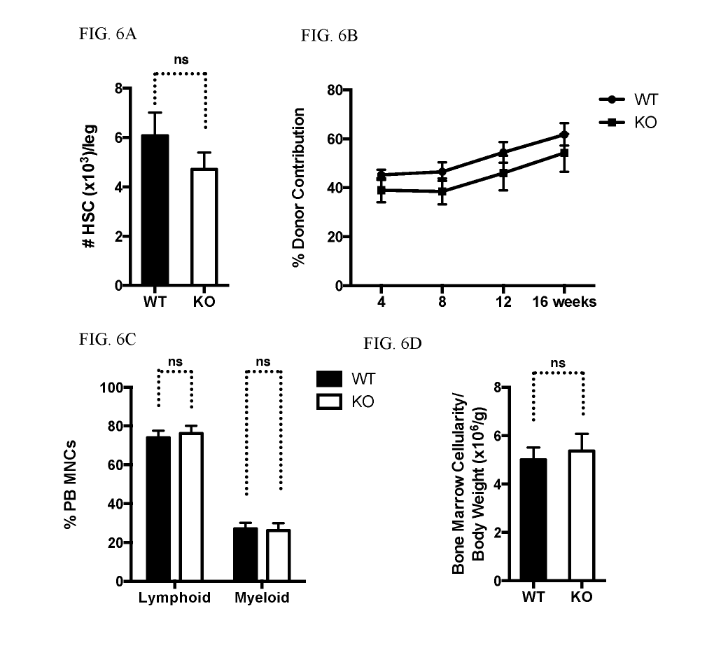

The expression of SIRT2 was reduced with age in HSCs (FIG. 5A-5C; and²²). This observation prompted us to investigate the role of SIRT2 in HSC aging. HSCs in wild type (WT) and SIRT2 knockout (KO) mice at a young (3-month-old) or an old (24-month-old) age were compared. SIRT2 KO mice are born at the Mendelian ratio and are phenotypically normal^{23,24}. Under homeostatic conditions, no difference in the number of immunophenotypically defined highly enriched HSCs (Lin c-Kit⁺ Sca1⁺CD150⁺CD48) was observed in the bone marrow of young WT and SIRT2 KO mice (FIG. 6A). HSCs isolated from young WT and SIRT2 KO mice were equally adept in reconstituting the blood system of lethally irradiated recipient mice in a competitive transplantation assay (FIG. 6B). HSCs differentiate into all blood cell types, including lymphoid and myeloid lineages. No significant difference was observed in the percentage of lymphoid cells (B220⁺ and CD3⁺) and myeloid cells (Mac-1⁺Gr1⁺) in the peripheral blood of WT and SIRT2 KO mice (FIG. 6C). Bone marrow cellularity was comparable between the two genotypes (FIG. 6D).

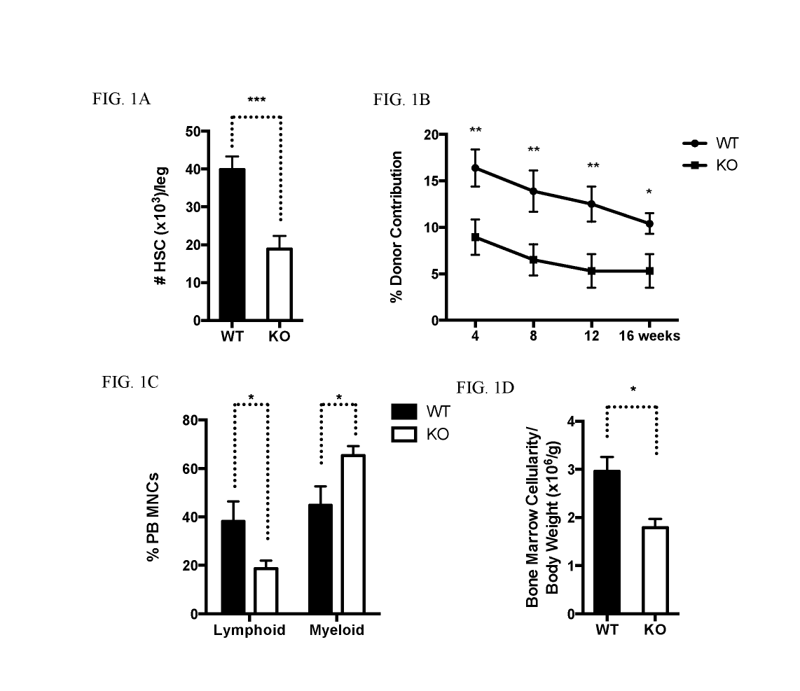

However, at 24 months old, the number of HSCs in the bone marrow of SIRT2 KO mice was reduced by 2-fold compared to their WT littermates under homeostatic condition (FIG. 1A). The ability of HSCs isolated from aged SIRT2 KO mice to reconstitute the blood system of lethally irradiated recipient mice also decreased by 2-fold compared to their WT counterparts (FIG. 1B). In the peripheral blood of aged SIRT2 KO mice, the percentage of lymphoid cells was reduced and the percentage of myeloid cells was increased (FIG. 1C). The bone marrow cellularity was reduced in aged SIRT2 KO mice (FIG. 1D). Thus, SIRT2 is required for HSC maintenance at an old age but not a young age.

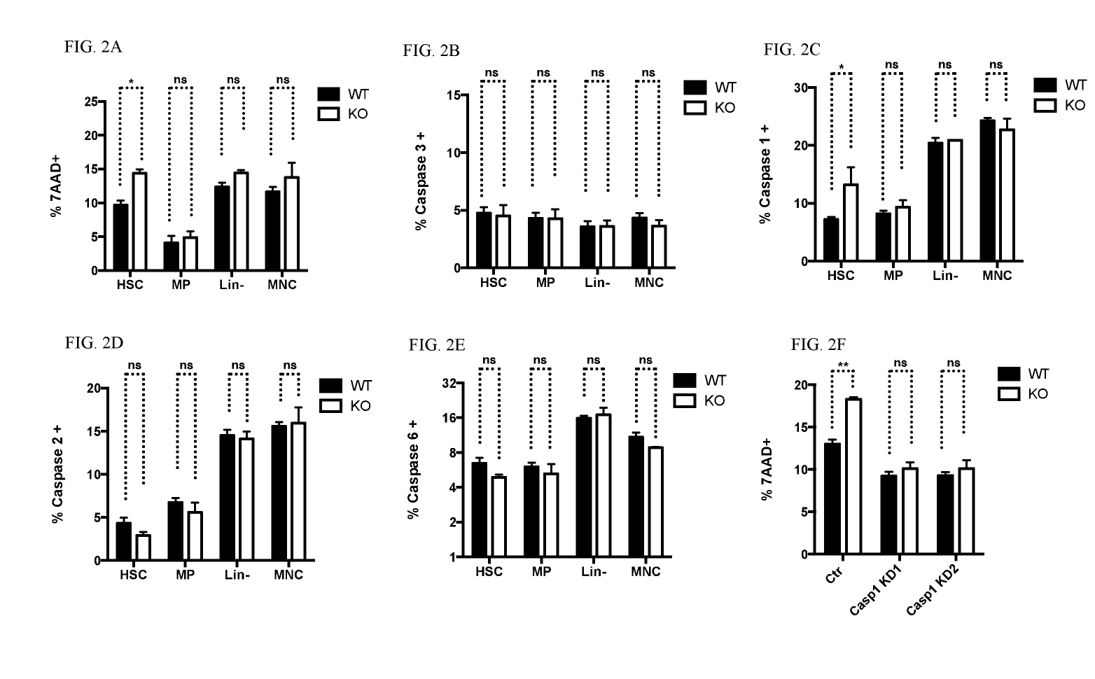

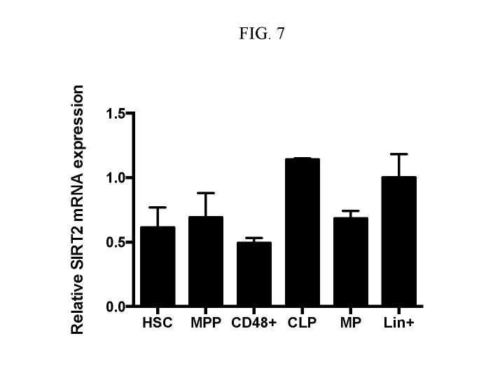

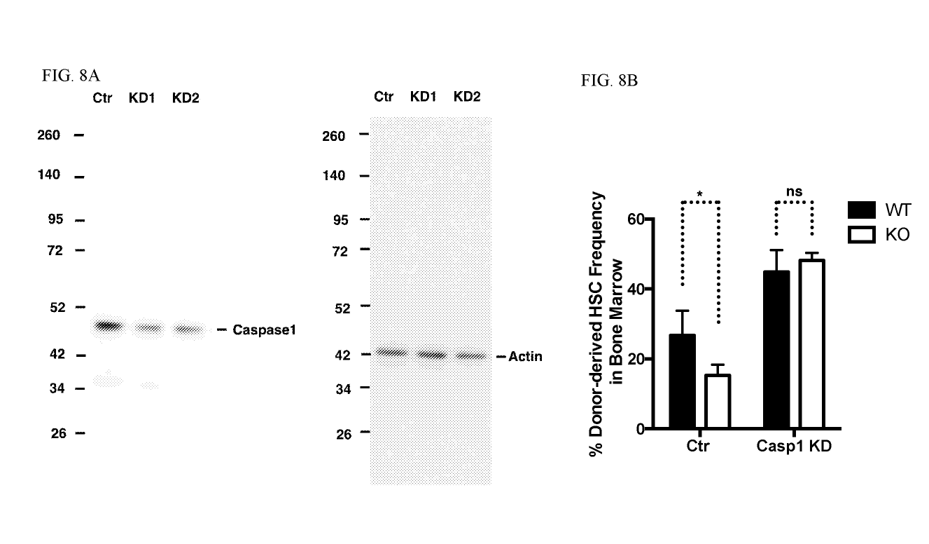

The manner by which SIRT2 promotes HSC maintenance was next examined. 7-Aminoactinomycin D (7AAD) viability staining showed increased death of aged SIRT2 KO HSCs compared to WT controls (FIG. 2A). However, WT and SIRT2 KO HSCs showed comparable activated caspase 3 staining (FIG. 2B). The activation of a panel of effector caspases was screened using fluorescently labeled inhibitor of caspases (FLICA) probes that target activated caspases with high specificity²⁵⁻²⁸. SIRT2 KO HSCs exhibited increased activation of caspase 1, which can trigger pyroptosis, a form of programmed cell death that is caspase 1 dependent by definition and is independent of apoptotic caspases 7 (FIG. 2C). In addition to caspase 3, the activity of other apoptotic caspases, such as caspase 2 and caspase 6, was unchanged (FIG. 2D, FIG. 2E). Increased cell death and caspase 1 activation resulting from SIRT2 deficiency were specific to the HSC compartment but not the differentiated populations (FIG. 2A, FIG. 2C). SIRT2 is ubiquitously expressed in various subpopulations in the hematopoietic system (FIG. 7). However, HSCs have greatly reduced levels of NADH²⁹, a competitive inhibitor of sirtuins³⁰. This may lead to increased SIRT2 activity in the HSC compartment. To determine whether SIRT2 KO HSCs die due to caspase 1 activation, the expression of caspase 1 was silenced via two independent short hairpin (shRNA) lentiviral vectors that specifically target caspase 1. Caspase 1 inactivation rescued the increased cell death of SIRT2 KO HSCs (FIG. 2F and FIG. 8). Thus, SIRT2 KO HSCs die via caspase 1-mediated pyroptosis.

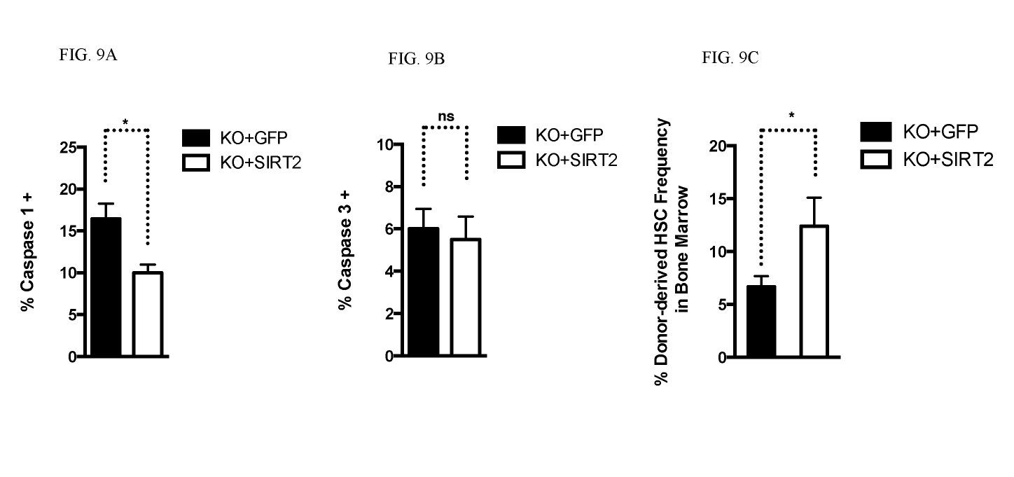

To determine whether SIRT2 controls HSC maintenance cell-autonomously or non-autonomously, i.e. whether SIRT2 regulates the HSC microenvironment or the niche, SIRT2 was overexpressed in aged SIRT2 KO HSCs via lentiviral transduction. Reintroduction of SIRT2 in aged SIRT2 KO HSCs repressed activation of caspase 1, but had no effect on the activation of caspase 3 (FIG. 9). Thus, SIRT2 promotes HSC survival cell-autonomously.

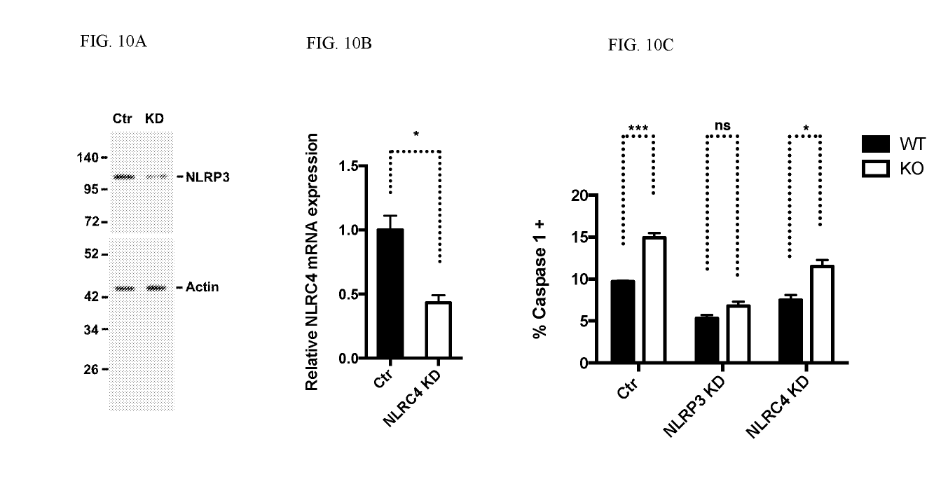

Next, how SIRT2 inhibits pyroptosis in HSCs was investigated. In the innate immune system, caspase 1 together with a subset of intracellular pattern recognition receptors, such as the nucleotide-binding domain and leucine-rich repeat-containing (NLR) proteins, assemble cytosolic protein complexes called inflammasomes to activate caspase 1³¹⁻³³. The expression of NLR proteins in HSCs isolated from WT and SIRT2 KO mice was silenced via shRNA lentiviral vectors that specifically target NLR proteins³⁴. Increased caspase 1 activation in SIRT2 KO HSCs persisted in the presence of NLRC4 inactivation but was blunted upon NLRP3 inactivation (FIG. 10), indicating that NLRP3 is required for SIRT2 to prevent pyroptosis in HSCs.

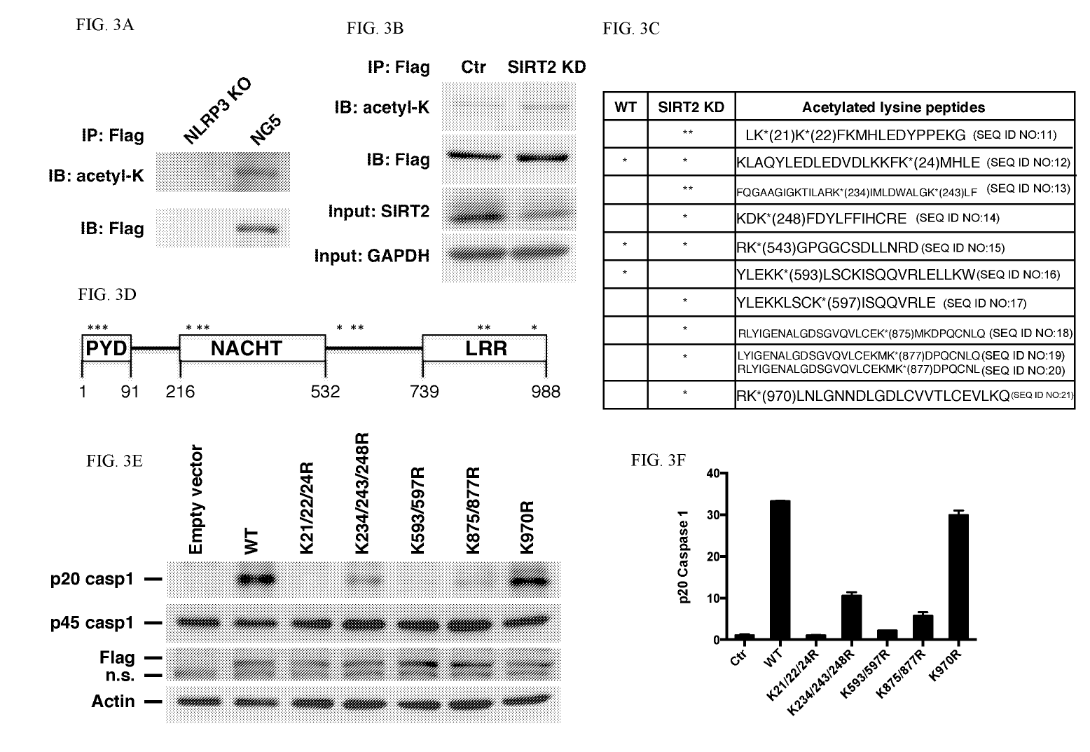

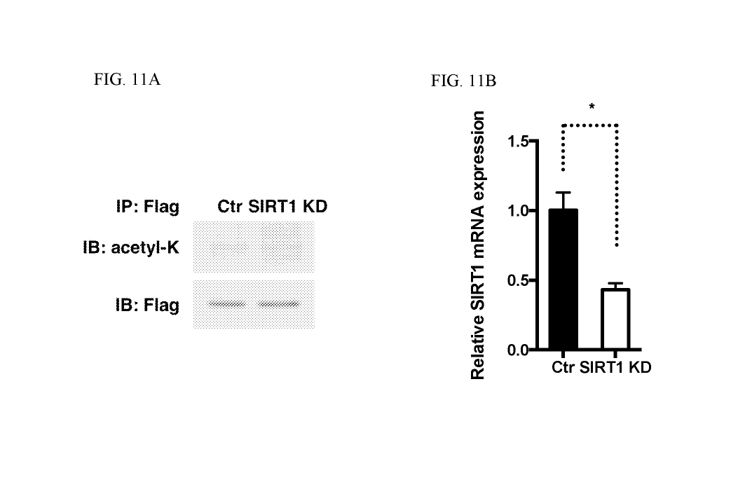

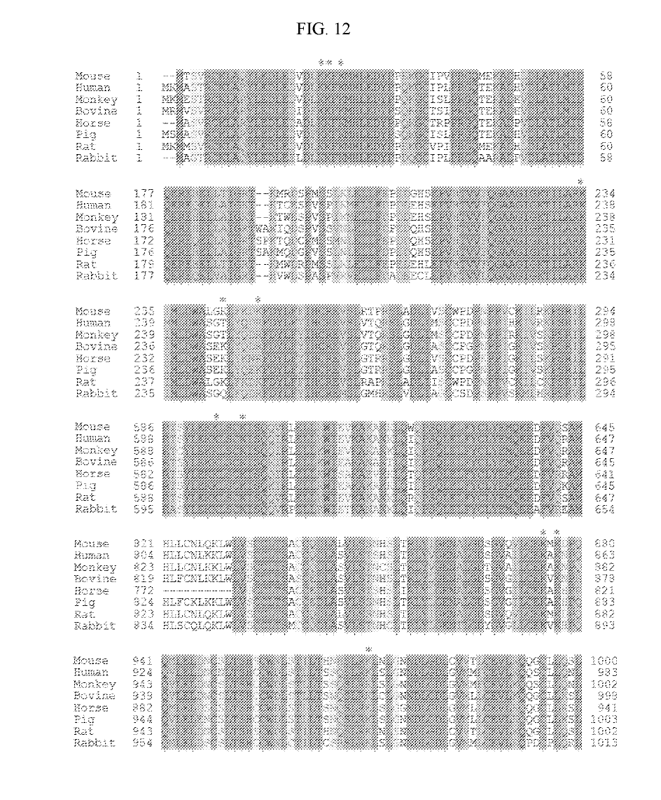

Because SIRT2 has deacetylase activity, the possibility that NLRP3 is modified by acetylation, which is subject to the regulation by SIRT2, was tested. Western analyses of immunopurified Flag-tagged NLRP3 isolated from NG5 cells, an immortalized NLRP3 KO macrophage line stably expressing Flag-NLRP3³⁵, showed that NLRP3 was modified by acetylation in vivo and its acetylation level was increased upon SIRT2 knockdown (KD) via small interfering RNA (siRNA) (FIG. 3A, FIG. 3B). In contrast, the acetylation level of NLRP3 was not affected by SIRT1 inactivation (FIG. 11). Mass spectrometry analyses of immunopurified Flag-tagged NLRP3 identified extensive lysine residues as being acetylated in SIRT2 KD cells but markedly reduced number of acetylated lysine residues in control cells (FIG. 3C). Spread out across the three major functional domains of NLRP3 (PYD, NACHT, and LRR) (FIG. 3D), these lysine residues targeted for acetylation are highly conserved in mammals (FIG. 12).

To investigate whether NLRP3 acetylation is required for inflammasome function, the lysine residues at the acetylation sites of NLRP3 were mutated to arginine to mimic the constitutively deacetylated state and reconstituted NLRP3 KO macrophages with WT or mutant forms of NLRP3 via retroviral transduction. WT NLRP3 and NLRP3 mutants were expressed to comparable levels (FIG. 3E).

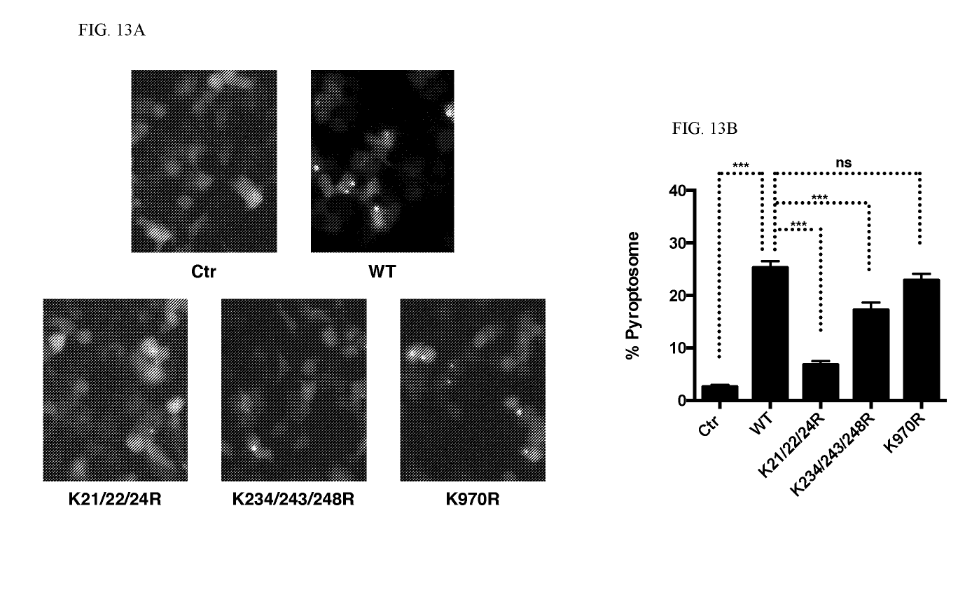

Upon priming with lipopolysaccharides (LPS) followed by ATP stimulation, cells reconstituted with WT NLRP3 processed caspase 1 (FIG. 3E, FIG. 3F). In contrast, cells reconstituted with NLRP3 mutants, except for NLRP3 K970R, had significantly reduced or non-detectable level of caspase 1 cleavage (FIG. 3E, 3F). In contrast to WT NLRP3 or K970R NLRP3 mutant, which trigger pyroptosome formation, NLRP3 mutants that failed to trigger caspase 1 cleavage were compromised in pyroptosome formation (FIG. 13). Thus, SIRT2 deacetylates NLRP3 to prevent caspase 1 activation. Consistently, pharmacological inhibition of SIRT2 enhances caspase 1 activation in response to NLRP3 induction^{3b}.

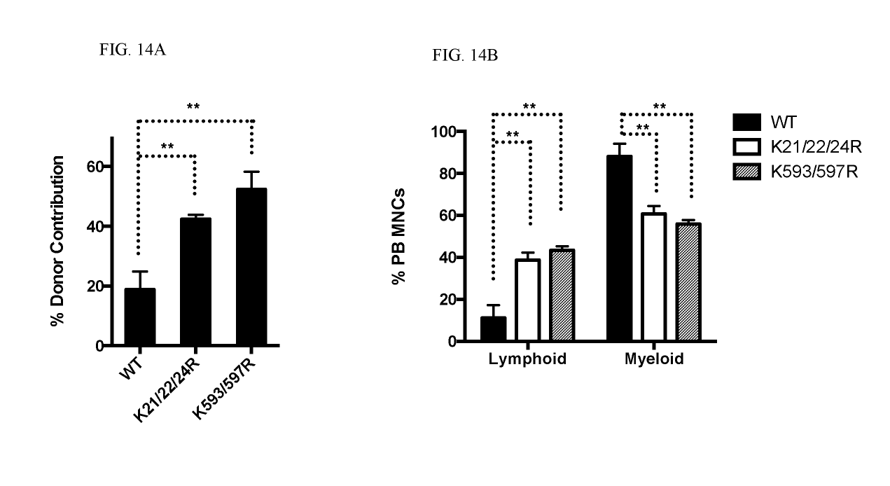

To determine whether NLRP3 deacetylation bypasses the requirement of SIRT2 for HSC maintenance, WT NLRP3 or constitutively deacetylated NLRP3 mutants were overexpressed in HSCs isolated from aged SIRT2 KO mice via retroviral transduction. Compared to WT NLRP3, constitutively deacetylated NLRP3 mutants exhibited increased reconstitution capacity and improved differentiation toward lymphoid lineage (FIG. 14).

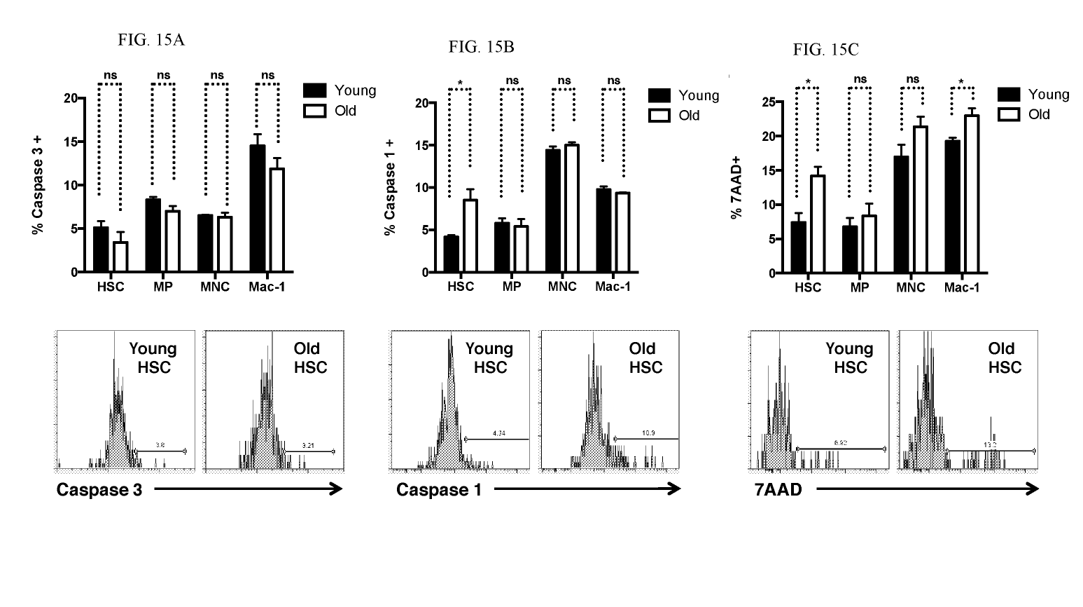

Reduced SIRT2 expression in aged HSCs suggests that HSC aging might be driven by induction of pyroptosis. The activation of effector caspases were assessed in HSCs of young and old WT mice. Under homeostatic condition, no difference in the activation of caspase 3 was observed in young and old HSCs (FIG. 15A), consistent with a previous report¹⁸. However, aged HSCs exhibited an increased activation of caspase 1 and reduced viability compared to young HSCs (FIG. 15B-15C). In contrast, there was no difference in caspase 1 activation in the differentiated subpopulations in the bone marrow of young and old mice.

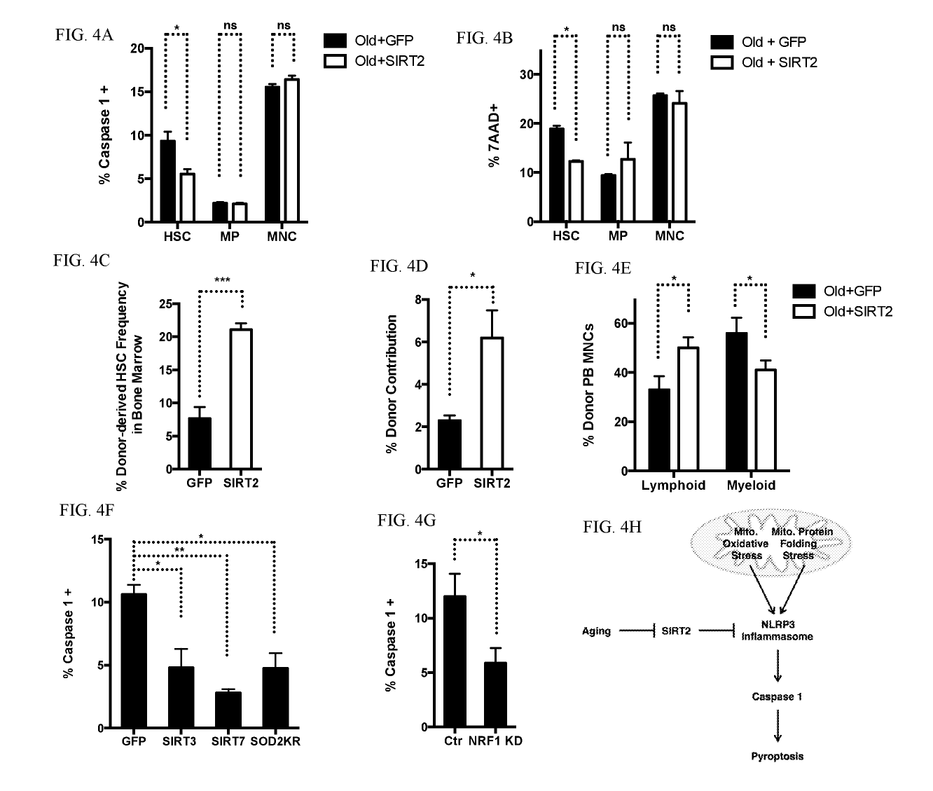

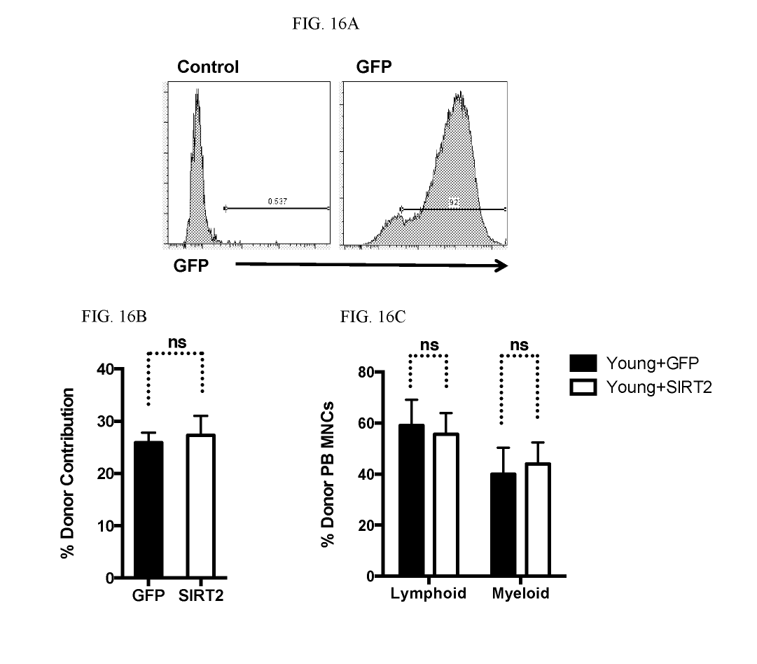

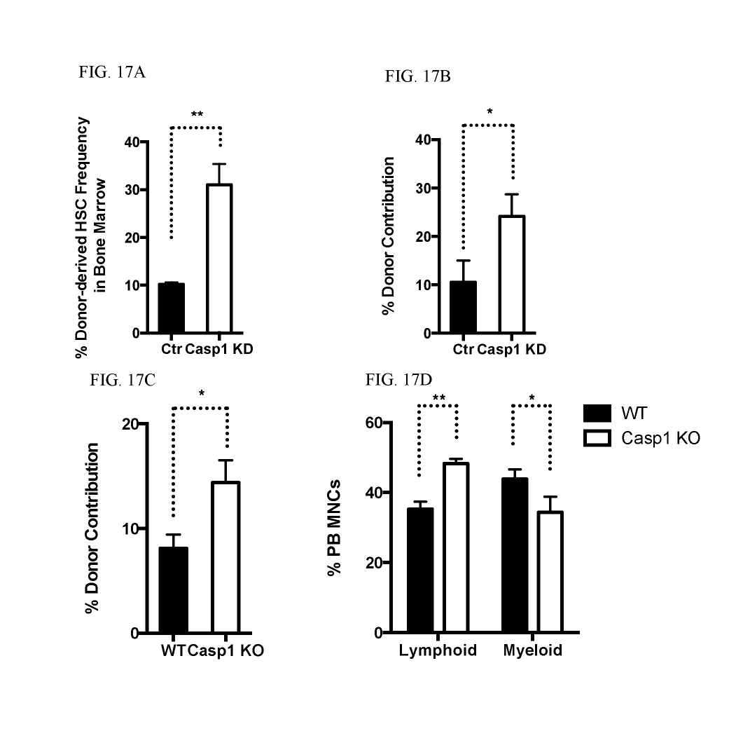

To determine whether repression of SIRT2 and activation of pyroptosis are causal to the deterioration of aged HSCs, SIRT2 was overexpressed or caspase 1 was knocked down in aged WT HSCs via lentiviral transduction. Reintroduction of SIRT2 in aged HSCs resulted in inactivation of caspase 1, improved cellular viability, increased HSC engraftment and reconstitution capacity, and reversed myeloid biased differentiation (FIG. 4A-4E). In contrast, SIRT2 overexpression did not affect young HSCs (FIG. 16). Caspase 1 inactivation in aged HSCs via shRNA increased HSC engraftment and reconstitution capacity (FIG. 17A-17B). Furthermore, HSCs from aged caspase 1 KO mice showed increased reconstitution capacity and ameliorated myeloid-biased differentiation (FIG. 17C-17D).

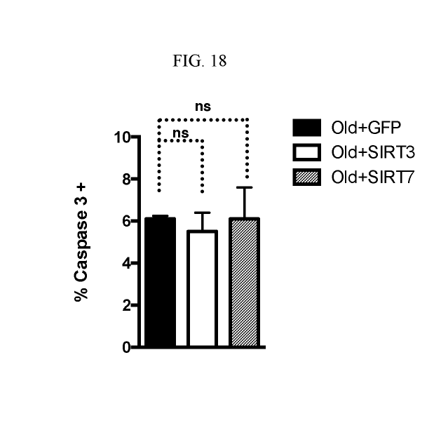

The NLRP3 inflammasome is unique among innate immune sensors, because it can be activated by endogenous signals in the absence of overt infection^{37,38}. Prominently, in macrophages, mitochondria play an essential role in NLRP3 inflammasome activation by providing a platform for assembling the NLRP3 inflammasome complex and housing the effector molecules that directly activate the NLRP3 inflammasome³⁹. Because mitochondrial stresses, such as mitochondrial oxidative stress^{23,49} and mitochondrial protein folding stress⁶, increase with age and have been implicated as causes of HSC aging, whether mitochondrial stress is a trigger of pyroptotic stimuli in aged HSCs was assessed. SIRT3, a mitochondrial deacetylase, promotes HSC maintenance by deacetylating two critical lysine residues on the mitochondrial antioxidant SOD2, promoting the enzymatic activity of SOD2, and reducing mitochondrial oxidative stress^{2,41}, while SIRT7, a histone deacetylase, enhances HSC maintenance by repressing the activity of the mitochondrial regulator nuclear respiratory factor 1 (NRF1) and suppressing mitochondrial protein folding stress⁶. Overexpression of SIRT3 or a constitutively active SOD2 mutant (SOD2 K53/89R) in aged HSCs via lentiviral transduction reduced ROS levels² and caspase 1 activation (FIG. 4F), but had no effect on caspase 3 activation (FIG. 18). SIRT7 overexpression or NRF1 KD in aged HSCs reduced mitochondrial protein folding stress⁶ and caspase 1 activation (FIG. 4F-4G), but not caspase 3 activation (FIG. 18). Thus, mitochondrial stress initiates pyroptosis in aged HSCs.

FIG. 1A-1D depict SIRT2 is required for HSC maintenance at an old age. FIG. 1A shows flow cytometry analyses showing reduced number of HSCs in the bone marrow of 24-month-old SIRT2 KO mice compared to WT controls. n=6. FIG. 1B shows competitive transplantation using HSCs isolated from 24-month-old WT and SIRT2 KO mice as donors showing reduced reconstitution capacity of SIRT2 KO HSCs in the peripheral blood (PB) of recipient mice. n=15. FIG. 1C shows myeloid-biased differentiation in the PB of 24-month-old SIRT2 KO mice. MNCs: mononuclear cells. n=6. FIG. 1D shows reduced bone marrow cellularity in 2-year-old SIRT2 KO mice. n=6. Data are biological replicates (a-d). Representative of two experiments (FIG. 1A, FIG. 1D). Error bars represent SE. *: p<0.05. **: p<0.01. ***: p<0.001. Student's t test.

FIG. 2A-2F depict that SIRT2 prevents pyroptosis in aged HSCs. FIG. 2A-2E shows staining for 7AAD and activated caspases showing reduced viability of aged SIRT2 KO HSCs due to caspase 1-mediated pyroptosis but not apoptosis. n=6. FIG. 2F shows caspase 1 was inactivated via shRNA in aged WT and SIRT2 KO HSCs. 7AAD staining showing reduced viability of SIRT2 KO HSCs is rescued by caspase 1 inactivation. n=6. Data are biological replicates (FIG. 2A-2E) and technical replicates (FIG. 2F) shows representative of two experiments (FIG. 2A-2F). Error bars represent SE. *: p<0.05. **: p<0.01. ns: p>0.05. Student's t test (FIG. 2A-F) and 2-way ANOVA (FIG. 2F)

FIG. 3A-3F depict analyses of immunopurified NLRP3-Flag from NG5 cells showing NLRP3 is acetylated in cells. NLRP3 KO cells were used as a negative control (FIG. 3A). FIG. 3B shows western analyses of immunopurified NLRP3-Flag from NG5 cells showing the acetylation level of NLRP3 is increased by SIRT2 siRNA treatment. FIG. 3C shows mass spectrometry analyses of immunopurified NLRP3-Flag identifying extensive acetylated lysine residues. FIG. 3D shows the domain structure of NLRP3. The acetylated lysine residues are marked as shown in FIG. 3D. FIG. 3E shows western analyses showing reduced caspase 1 cleavage in NLRP3 KO macrophages reconstituted with NLRP3 mutants compared to WT NLRP3 control upon stimulation with LPS and ATP. FIG. 3F shows quantification of stimulation with LPS and ATP. Representative of three (FIG. 3A, FIG. 3B) or two experiments (FIG. 3E).

FIG. 4A-H depict mitochondrial stress-initiated caspase 1-mediated pyroptosis regulates HSC aging. FIG. 4A-4B show staining for 7AAD and activated caspase 1 showing overexpression of SIRT2 via lentiviral transduction improves the viability of aged HSCs. FIG. 4C-4E show competitive transplantation using aged HSCs transduced with SIRT2 or control lentivirus as donors. SIRT2 overexpression in aged HSCs increases HSC engraftment in the bone marrow (FIG. 4C) and reconstitution capacity (FIG. 4D) and reverses myeloid-biased differentiation (FIG. 4E) in the peripheral blood. n=6. FIG. 4F-4G show staining for activated caspase 1 showing overexpression of SIRT3, SIRT7, or SOD2 K53/89R mutant, or NRF1 shRNA KD via lentiviral transduction reduces caspase 1 activation in aged HSCs. n=3. FIG. 4H shows a proposed model where SIRT2 represses NLRP3 inflammasome activation by deacetylating NLRP3. In aged HSCs, reduced SIRT2 expression and increased mitochondrial stresses lead to activation of NLRP3 inflammasome and induction of caspase 1-mediated pyroptosis. Data are biological replicates (FIG. 4A-4E) or technical replicates (FIG. 4F-4G). Representative of two experiments (FIG. 4A-4G). Error bars represent SE. *: p<0.05. **: p<0.01. ***: p<0.001. ns: p>0.05. Student's t test.

FIG. 5A-5C depict reduction of SIRT2 expression with age in HSCs. FIG. 5A shows the gating strategy. Lin depicts lineage negative cells, and LKS depicts Lin c-Kit⁺ Sca1⁺ cells, and MP depicts myeloid progenitor cells. FIG. 5B-C shows the expression of SIRT2 in HSCs isolated from young (3 months old) and old mice (2 years old) quantified by qPCR (FIG. 5B, n=4) or detected by immunocytochemistry (FIG. 5C, n=3). Blue: grey; Green: white. Data are biological replicates. Error bars represent SE. **: p<0.01. Student's t test.

FIG. 6A-6D depict lack of requirement for SIRT2 for HSC maintenance at a young age. FIG. 6A shows flow cytometry analysis showing comparable number of HSCs in the bone marrow of 3-month-old WT and SIRT2 KO mice under homeostatic condition (n=9). FIG. 6B shows competitive transplantation using HSCs isolated from 3-month-old WT and SIRT2 KO mice as donors showing comparable reconstitution capacity of SIRT2 KO HSCs in the peripheral blood of recipient mice (n-15). FIG. 3C shows that lineage differentiation in the peripheral blood of 3-months-old SIRT2 KO mice is unaffected. Mononuclear cells are depicted as MNCs (n=9). FIG. 6D shows normal bone marrow cellularity in 3-month-old SIRT2 KO mice (n=9). Data are biological replicates. Representative of two (FIG. 6A, FIG. 6C, FIG. 6D) or three (FIG. 6B) experiments. Error bars represent SE. ns: p>0.05. Student's t test.

FIG. 7 depicts expression of SIRT2 in various hematopoietic cellular compartments in the bone marrow. Various cell populations in the bone marrow were isolated via cell sorting based on cell surface markers. The cell populations include HSCs denoted as Lin c-Kit⁺ Sca1⁺CD150⁺CD48⁺; multipotent progenitors denoted as (MPPs), Lin c-Kit⁺ Sca1⁺CD150 CD48; CD48⁺, Lin c-Kit⁺ Sca1⁺CD48+; CLP, Lin-IL7Rα⁺c-kit^{med}/Sca1^{med}; myeloid progenitors (MPs), Lin c-Kit⁺ Sca1⁻; and differentiated blood cells denoted as Lin⁺. The expression of SIRT2 was determined by qPCR. n=3. Data are biological replicates.

FIG. 8A-8B shows that SIRT2 prevents pyroptosis in aged HSCs. FIG. 8A shows that NG5 cells were transduced with control or two independent shRNA knockdown lentiviral constructs for caspase 1. Gene expression was determined by western analyses. FIG. 8B shows competitive transplantation using aged WT or SIRT2 KO HSCs transduced with control or caspase 1 shRNA lentivirus as donors. Data shown are percentage of donor-derived HSCs in the bone marrow of recipient mice. Error bars represent SE. *: p<0.05. ns: p>0.05. Student's t test.

FIG. 9A-9C depict SIRT2 regulation of HSCs cell-autonomously. Aged SIRT2 KO HSCs transduced with SIRT2 or control lentivirus were used as donors in a competitive transplantation assay. Data shown are caspase 1 (FIG. 9A) and caspase 3 activation (FIG. 9B) in HSCs, and the percentage of donor-derived HSC contribution in the bone marrow of the recipients (FIG. 9C). n=6. Data are biological replicates. Representative of two experiments. Error bars represent SE. *: p<0.05. Student's t test.

FIG. 10A-10C depict requirement for NLRP3 for SIRT2 repression of caspase-1 activation. NG5 cells were transduced with control or shRNA knockdown lentivirus for NLRP3 (FIG. 10A) or NLRC4 (FIG. 10B). FIG. 10A shows gene expression determined by western analyses. FIG. 10B shows gene expression determined by qPCR. FIG. 10C shows that NLRP3 or NLRC4 were inactivated via shRNA lentiviral vectors in HSCs isolated from WT and SIRT2 KO mice. Activated caspase 1 staining showing NLRP3 is required for SIRT2 to repress caspase 1 activation. n=3. Data are technical replicates. Representative of two experiments. Error bars represent SE. *: p<0.05. ***: p<0.001. ns: p>0.05. Student's t test.

FIG. 11A-11B depict lack of effect of SIRT1 on NLRP3 acetylation. FIG. 11A shows western analyses of immunopurified NLRP3-Flag from NG5 cells treated with control or SIRT 1 siRNA showing the acetylation level of NLRP3 is not affected by SIRT1 inactivation. FIG. 11B shows validation of SIRT1 siRNA knockdown efficiency. Data shown are the SIRT1 mRNA levels in NG5 cells treated with control or SIRT1 siRNA. n=3. Data are technical replicates. Representative of two experiments. Error bars represent SE. *: p<0.05. Student's t test.

FIG. 12 provides an alignment of NLRP3 amino acid sequences from various species: Mouse (SEQ ID NO:22), Human (SEQ ID NO:23), Monkey (SEQ ID NO:24), Bovine (SEQ ID NO:25), Horse (SEQ ID NO:26), Pig (SEQ ID NO:27), Rat (SEQ ID NO:28), Rabbit (SEQ ID NO:29). The acetylation sites on NLRP3 are conserved across species. Sequence alignment of NLRP3 from various species is shown. Acetylated lysine residues are labeled with *.

FIG. 13A-13B depict the effect of NLRP3 acetylation on pyroptosome formation. 293T cells were co-transfected with ASC-EGFP and control vector or WT or constitutively deacetylated mutant NLRP3. FIG. 13A shows the fluorescence images and FIG. 13B shows quantification of formation of speck-like pyroptosome. Data are technical replicates. Representative of two experiments. Error bars represent SE. ***: p<0.001. ns: p>0.05. Student's t test.

FIG. 14A-14B depict the effect of NLRP3 deacetylation on the functionality of aged HSCs, where deacetylation of NLRP3 improves the functionality of aged HSCs. Competitive transplantation using aged SIRT2 KO HSCs transduced with WT NLRP3 or constitutively deacetylated NLRP3 mutant retrovirus as donors showing constitutively deacetylated NLRP3 improves the reconstitution capacity and ameliorates myeloid-biased differentiation of aged HSCs. FIG. 14A shows the percentage of donor-derived cells in the peripheral blood of the recipients and FIG. 14B shows donor-derived lineage differentiation in the peripheral blood (n=6). Data are biological replicates. Representative of two experiments. Error bars represent SE. **: p<0.01. Student's t test.

FIG. 15A-15C depict induction of pyroptosis in aged HSCs. FIG. 15A-15B depicts staining for activated caspases and FIG. 15C depicts staining of 7AAD, showing reduced viability of aged HSCs compared to young HSCs due to caspase 1-mediated pyroptosis but not caspase 3-mediated apoptosis. Mac-1: Mac-1⁺F4/80⁺. n=4. Data are biological replicates. Representative of two experiments. Error bars represent SE. *: p<0.05. ns: p>0.05. Student's t test.

FIG. 16A-16C depict the effect of SIRT2 overexpression on young HSCs, where SIRT2 overexpression does not affect young HSCs. FIG. 16A shows that lentiviral transduction efficiency was quantified based on GFP expression. FIG. 16B-16C shows competitive transplantation using young HSCs transduced with SIRT2 or control lentivirus as donors. FIG. 16B shows the percentage of donor-derived cells in the peripheral blood of the recipients and FIG. 16C shows the donor-derived lineage differentiation in the peripheral blood (n=6). Data are biological replicates. Error bars represent SE. ns: p>0.05. Student's t test.

FIG. 17A-17D depict regulation of HSC aging by caspase-1. FIG. 17A-17B show competitive transplantation using aged HSCs transduced with caspase 1 shRNA lentivirus or control virus as donors showing caspase 1 inactivation increases HSC engraftment and reconstitution capacity of aged HSCs. FIG. 17A shows the percentage of donor-derived HSCs in the bone marrow of the recipients and FIG. 17B shows the percentage of donor-derived cells in the peripheral blood of the recipients (n=6). FIG. 17C shows competitive transplantation using HSCs from aged WT and caspase 1 KO mice as donors. FIG. 17D shows the percentage of donor-derived cells in the peripheral blood of the recipients (n=16). FIG. 17D shows lineage differentiation in the peripheral blood of aged WT and Caspase 1 KO mice (n=6). Data are biological replicates. Representative of two experiments. Error bars represent SE. *: p<0.05. **: p<0.01. Student's t test.

FIG. 18 depicts the effect of SIRT3 and SIRT7 on caspase-3 activity in aged HSCs. Activated caspase 3 staining showing overexpression of SIRT3 or SIRT7 via lentiviral transduction does not affect caspase 3 activation in aged HSCs (n=3). Data are technical replicates. Representative of two experiments. Error bars represent SE. ns: p>0.05. Student's t test.

FIG. 19 provides Table 1 and Table 2 showing a list of antibodies and reagents used in Example 1.

FIG. 20 provides Table 3 showing primers used for qPCR analysis in Example 1.

### REFERENCES

1 Kowald, A. & Kirkwood, T. B. Evolutionary significance of ageing in the wild. Experimental gerontology, doi:10.1016/j.exger.2015.08.006 (2015).
2 Brown, K. et al. SIRT3 reverses aging-associated degeneration. Cell reports 3, 319-327, doi:10.1016/j.celrep.2013.01.005 (2013).
3 Katajisto, P. et al. Stem cells. Asymmetric apportioning of aged mitochondria between daughter cells is required for stemness. Science 348, 340-343, doi:10.1126/science.1260384 (2015).
4 Luchsinger, L. L., de Almeida, M. J., Corrigan, D. J., Mumau, M. & Snoeck, H. W. Mitofusin 2 maintains haematopoietic stem cells with extensive lymphoid potential. Nature 529, 528-531, doi:10.1038/nature16500 (2016).
5 Mohrin, M. & Chen, D. The mitochondrial metabolic checkpoint and aging of hematopoietic stem cells. Current opinion in hematology, doi:10.1097/MOH.0000000000000244 (2016).
6 Mohrin, M. et al. Stem cell aging. A mitochondrial UPR-mediated metabolic checkpoint regulates hematopoietic stem cell aging. Science 347, 1374-1377, doi:10.1126/science.aaa2361 (2015).
7 Bergsbaken, T., Fink, S. L. & Cookson, B. T. Pyroptosis: host cell death and inflammation. Nature reviews. Microbiology 7, 99-109, doi:10.1038/nrmicro2070 (2009).
8 Fink, S. L. & Cookson, B. T. Apoptosis, pyroptosis, and necrosis: mechanistic description of dead and dying eukaryotic cells. Infection and immunity 73, 1907-1916, doi:10.1128/IAI.73.4.1907-1916.2005 (2005).
9 Lamkanfi, M. & Dixit, V. M. Manipulation of host cell death pathways during microbial infections. Cell host &microbe 8, 44-54, doi:10.1016/j.chom.2010.06.007 (2010).
10 Geiger, H., de Haan, G. & Florian, M. C. The ageing haematopoietic stem cell compartment. Nature reviews. Immunology 13, 376-389, doi:10.1038/nri3433 (2013).
11 Lopez-Otin, C., Blasco, M. A., Partridge, L., Serrano, M. & Kroemer, G. The hallmarks of aging. Cell 153, 1194-1217, doi:10.1016/j.cell.2013.05.039 (2013).
12 Oh, J., Lee, Y. D. & Wagers, A. J. Stem cell aging: mechanisms, regulators and therapeutic opportunities. Nature medicine 20, 870-880, doi:10.1038/nm.3651 (2014).
13 Janzen, V. et al. Stem-cell ageing modified by the cyclin-dependent kinase inhibitor p16INK4a. Nature 443, 421-426, doi:10.1038/nature05159 (2006).
14 Rossi, D. J., Jamieson, C. H. & Weissman, I. L. Stems cells and the pathways to aging and cancer. Cell 132, 681-696, doi:10.1016/j.cell.2008.01.036 (2008).
15 Warr, M. R. et al. FOXO3A directs a protective autophagy program in haematopoietic stem cells. Nature 494, 323-327, doi:10.1038/nature 1895 (2013).
16 Imai, S., Armstrong, C. M., Kaeberlein, M. & Guarente, L. Transcriptional silencing and longevity protein Sir2 is an NAD-dependent histone deacetylase. Nature 403, 795-800, doi:10.1038/35001622 (2000).
17 Kaeberlein, M., McVey, M. & Guarente, L. The SIR2/3/4 complex and SIR2 alone promote longevity in Saccharomyces cerevisiae by two different mechanisms. Genes &development 13, 2570-2580 (1999).
18 Kawahara, T. L. et al. SIRT6 links histone H3 lysine 9 deacetylation to NF-kappaB-dependent gene expression and organismal life span. Cell 136, 62-74, doi:10.1016/j.cell.2008.10.052 (2009).
19 Kennedy, B. K., Austriaco, N. R., Jr., Zhang, J. & Guarente, L. Mutation in the silencing gene SIR4 can delay aging in S. cerevisiae. Cell 80, 485-496 (1995).
20 Michishita, E. et al. SIRT6 is a histone H3 lysine 9 deacetylase that modulates telomeric chromatin. Nature 452, 492-496, doi:10.1038/nature06736 (2008).
21 Mostoslavsky, R. et al. Genomic instability and aging-like phenotype in the absence of mammalian SIRT6. Cell 124, 315-329, doi:10.1016/j.cell.2005.11.044 (2006).
22 Chambers, S. M. et al. Aging hematopoietic stem cells decline in function and exhibit epigenetic dysregulation. PLoS biology 5, e201, doi:10.1371/journal.pbio.0050201 (2007).
23 Bobrowska, A., Donmez, G., Weiss, A., Guarente, L. & Bates, G. SIRT2 ablation has no effect on tubulin acetylation in brain, cholesterol biosynthesis or the progression of Huntington's disease phenotypes in vivo. PloS one 7, e34805, doi:10.1371/journal.pone.0034805 (2012).
24 Lo Sasso, G. et al. SIRT2 deficiency modulates macrophage polarization and susceptibility to experimental colitis. PloS one 9, e103573, doi:10.1371/journal.pone.0103573 (2014).
25 Bedner, E., Smolewski, P., Amstad, P. & Darzynkiewicz, Z. Activation of caspases measured in situ by binding of fluorochrome-labeled inhibitors of caspases (FLICA): correlation with DNA fragmentation. Experimental cell research 259, 308-313, doi: 10.1006/excr.2000.4955 (2000).
26 Bruchard, M. et al. The receptor NLRP3 is a transcriptional regulator of TH2 differentiation. Nature immunology 16, 859-870, doi:10.1038/ni.3202 (2015).
27 Doitsh, G. et al. Cell death by pyroptosis drives CD4 T-cell depletion in HIV-1 infection. Nature 505, 509-514, doi:10.1038/nature12940 (2014).
28 Shi, H. et al. NLRP3 activation and mitosis are mutually exclusive events coordinated by NEK7, a new inflammasome component. Nature immunology 17, 250-258, doi:10.1038/ni.3333 (2016).
29 Simsek, T. et al. The distinct metabolic profile of hematopoietic stem cells reflects their location in a hypoxic niche. Cell stem cell 7, 380-390, doi:10.1016/j.stem.2010.07.011 (2010).
30 Lin, S. J., Ford, E., Haigis, M., Liszt, G. & Guarente, L. Calorie restriction extends yeast life span by lowering the level of NADH. Genes &development 18, 12-16, doi:10.1101/gad.1164804 (2004).
31 Lamkanfi, M. & Dixit, V. M. Mechanisms and functions of inflammasomes. Cell 157, 1013-1022, doi:10.1016/j.cell.2014.04.007 (2014).
32 Schroder, K. & Tschopp, J. The inflammasomes. Cell 140, 821-832, doi:10.1016/j.cell.2010.01.040 (2010).
33 von Moltke, J., Ayres, J. S., Kofoed, E. M., Chavarria-Smith, J. & Vance, R. E. Recognition of bacteria by inflammasomes. Annual review of immunology 31, 73-106, doi:10.1146/annurev-immunol-032712-095944 (2013).
34 Lee, G. S. et al. The calcium-sensing receptor regulates the NLRP3 inflammasome through Ca2+ and cAMP. Nature 492, 123-127, doi:10.1038/naturell588 (2012).
35 Juliana, C. et al. Anti-inflammatory compounds parthenolide and Bay 11-7082 are direct inhibitors of the inflammasome. The Journal of biological chemistry 285, 9792-9802, doi:10.1074/jbc.M109.082305 (2010).
36 Misawa, T. et al. Microtubule-driven spatial arrangement of mitochondria promotes activation of the NLRP3 inflammasome. Nature immunology 14, 454-460, doi:10.1038/ni.2550 (2013).
37 Martinon, F., Mayor, A. & Tschopp, J. The inflammasomes: guardians of the body. Annual review of immunology 27, 229-265, doi: 10.1146/annurev.immunol.021908.132715 (2009).
38 Strowig, T., Henao-Mejia, J., Elinav, E. & Flavell, R. Inflammasomes in health and disease. Nature 481, 278-286, doi:10.1038/nature10759 (2012).
39 Wen, H., Miao, E. A. & Ting, J. P. Mechanisms of NOD-like receptor-associated inflammasome activation. Immunity 39, 432-441, doi:10.1016/j.immuni.2013.08.037 (2013).
40 Ito, K. et al. Reactive oxygen species act through p38 MAPK to limit the lifespan of hematopoietic stem cells. Nature medicine 12, 446-451, doi:10.1038/nm1388 (2006).
41 Qiu, X., Brown, K., Hirschey, M. D., Verdin, E. & Chen, D. Calorie restriction reduces oxidative stress by SIRT3-mediated SOD2 activation. Cell metabolism 12, 662-667, doi:10.1016/j.cmet.2010.11.015 (2010).
42 Pellegrino, M. W. et al. Mitochondrial UPR-regulated innate immunity provides resistance to pathogen infection. Nature 516, 414-417, doi:10.1038/nature13818 (2014).
43 West, A. P. et al. Mitochondrial DNA stress primes the antiviral innate immune response. Nature 520, 553-557, doi:10.1038/nature14156 (2015).
44 Dagenais, M., Skeldon, A. & Saleh, M. The inflammasome: in memory of Dr. Jurg Tschopp. Cell death and differentiation 19, 5-12, doi:10.1038/cdd.2011.159 (2012).
45 Williams, G. Pleiotropy, Natural Selection, and the Evolution of Senescence. Evolution 11, 398-411 (1957).
46 Fernandes-Alnemri, T., Yu, J. W., Datta, P., Wu, J. & Alnemri, E. S. AIM2 activates the inflammasome and cell death in response to cytoplasmic DNA. Nature 458, 509-513, doi:10.1038/nature07710 (2009).
47 Shin, J. et al. SIRT7 represses Myc activity to suppress ER stress and prevent fatty liver disease. Cell reports 5, 654-665, doi:10.1016/j.celrep.2013.10.007 (2013).
48 Miyamoto, K. et al. Foxo3a is essential for maintenance of the hematopoietic stem cell pool. Cell stem cell 1, 101-112, doi:10.1016/j.stem.2007.02.001 (2007).
49 Kuida, K. et al. Altered cytokine export and apoptosis in mice deficient in interleukin-1 beta converting enzyme. Science 267, 2000-2003 (1995).
50 Zhao, C. et al. Hedgehog signalling is essential for maintenance of cancer stem cells in myeloid leukaemia. Nature 458, 776-779, doi:10.1038/nature07737 (2009).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method for improving the function of an adult stem cell, the method comprising one or more of:
a) increasing the level and/or activity of a SIRT2 polypeptide in the adult stem cell;
b) increasing the deacetylase activity of a SIRT2 polypeptide in the adult stem cell;
c) increasing deacetylation of a nucleotide-binding domain and leucine-rich repeat-containing-3 (NLRP3) polypeptide in the adult stem cell;
d) reducing the level and/or activity of an NLRP3 polypeptide in the adult stem cell;
e) reducing the level and/or activity of a caspase-1 polypeptide in the adult stem cell; and
f) inhibiting pyroptosis in the adult stem cell.

2. The method of claim 1, wherein the adult stem cell is a muscle stem, a hematopoietic stem cell, an epithelial stem cell, a neural stem cell, a mesenchymal stem cell, a mammary stem cell, an intestinal stem cell, a mesodermal stem cell, an endothelial stem cell, an olfactory stem cell, or a neural crest stem cell.

3. The method of claim 1, wherein the adult stem cell is a rodent adult stem cell, a human adult stem cell, or a non-human primate adult stem cell.

4. The method of claim 1, wherein reduction of the level of an NLRP3 polypeptide in the adult stem cell comprises introduction into the adult stem cell of an inhibitory nucleic acid that specifically reduces the level of NLRP3 mRNA and/or NLRP3 polypeptide in the adult stem cell.

5. The method of claim 1, wherein the:
a) increase in the level and/or activity of the SIRT2 polypeptide,
b) increase in the deacetylase activity of the SIRT2 polypeptide,
c) increase in deacetylation of the NLRP3,
d) reduction in the level and/or activity of the NLRP3 polypeptide, or
e) reduction in the level and/or activity of the caspase-1 polypeptide in the adult stem cell is achieved by administration of:
(i) an activator of a SIRT selected from: a 1,4-dihydropyridine (DHP) derivative bearing a benzyl group at the N1 position, SRT1720 HCl, and Fisetin; or
(ii) an inhibitor of NLRP3 selected from: glyburide, 16673-34-0, MCC950, Shikonin, sodium butyrate, β-hydroxybutyrate, and an siRNA targeting NLRP3 mRNA; or
(iii) an inhibitor of caspase-1 selected from: z-VAD-fmk, ac-YVAD-cmk, VX-765, and z-WEHD-fmk; or
(iv) an agent that increases intracellular NAD⁺ levels selected from: a P2X7R receptor, extracellular NADH, CoQ10, LDH, G3PDH, MDH, NMN and NR.

6. The method of claim 4, wherein the inhibitory nucleic acid is a short interfering nucleic acid, a short hairpin RNA, a ribozyme, or an antisense nucleic acid.

7. The method of claim 6, wherein the inhibitory nucleic acid comprises one or more of a base modification, a backbone modification, a modified internucleoside linkage, and a modified sugar moiety.

8. The method of claim 1, wherein reduction of the level of the caspase-1 polypeptide in the adult stem cell comprises introduction into the adult stem cell of an inhibitory nucleic acid that specifically reduces the level of caspase-1 mRNA and/or caspase-1 polypeptide in the adult stem cell.

9. The method of claim 8, wherein the inhibitory nucleic acid is a short interfering nucleic acid, a short hairpin RNA, a ribozyme, or an antisense nucleic acid.

10. The method of claim 9, wherein the inhibitory nucleic acid comprises one or more of a base modification, a backbone modification, a modified internucleoside linkage, and a modified sugar moiety.

11. The method of claim 1, wherein reduction of the activity of the caspase-1 polypeptide in the adult stem cell comprises contacting the stem cell with a compound that inhibits caspase-1 activity.

12. The method of claim 1, wherein increasing the level of a SIRT2 polypeptide in the adult stem cell comprises introducing into the adult stem cell a nucleic acid comprising a nucleotide sequence encoding a SIRT2 polypeptide.

13. The method of claim 12, wherein the nucleotide sequence is operably linked to a promoter.

14. The method of claim 13, wherein the promoter is inducible.

15. The method of claim 12, wherein the nucleic acid is a recombinant expression vector.

16. The method of claim 15, wherein the expression vector is a recombinant viral vector.

17. A method of improving the function of an adult stem cell in an individual, the method comprising administering to the individual an agent that:
a) increases the level of a SIRT2 polypeptide in the adult stem cell;
b) increases the deacetylase activity of a SIRT2 polypeptide in the adult stem cell;
c) increases deacetylation of a nucleotide-binding domain and leucine-rich repeat-containing-3 (NLRP3) polypeptide in the adult stem cell;
d) reduces the level and/or activity of an NLRP3 polypeptide in the adult stem cell;
e) reduces the level and/or activity of a caspase-1 polypeptide in the adult stem cell; or
f) inhibits pyroptosis in the adult stem cell.

18. The method of claim 17, wherein the agent is:
(i) an activator of a SIRT selected from: a 1,4-dihydropyridine (DHP) derivative bearing a benzyl group at the N1 position, SRT1720 HCl, and Fisetin; or
(ii) an inhibitor of NLRP3 selected from: glyburide, 16673-34-0, MCC950, Shikonin, sodium butyrate, β-hydroxybutyrate, and an siRNA targeting NLRP3 mRNA;
(iii) an inhibitor of caspase-1 selected from: z-VAD-fmk, ac-YVAD-cmk, VX-765, and z-WEHD-fmk; or
(iv) an agent that increases intracellular NAD⁺ levels selected from: a P2X7R receptor, extracellular NADH, CoQ10, LDH, G3PDH, MDH, NMN and NR.

19. A method of improving the function of an adult stem cell in an individual, the method comprising:
i) carrying out, in vitro, one or more of:
a) increasing the level of a SIRT2 polypeptide;
b) increasing the deacetylase activity of a SIRT2 polypeptide;
c) increasing deacetylation of a nucleotide-binding domain and leucine-rich repeat-containing-3 (NLRP3) polypeptide;
d) reducing the level and/or activity of an NLRP3 polypeptide;
e) reducing the level and/or activity of a caspase-1 polypeptide; and
f) inhibiting pyroptosis;
in an adult stem cell obtained from the individual, thereby modifying the adult stem cell; and
ii) introducing the modified adult stem cell into the individual.

20. The method of claim 19, wherein the:
a) increase in the level and/or activity of the SIRT2 polypeptide,
b) increase in the deacetylase activity of the SIRT2 polypeptide,
c) increase in deacetylation of the NLRP3,
d) reduction in the level and/or activity of the NLRP3 polypeptide, or
e) reduction in the level and/or activity of the caspase-1 polypeptide in the adult stem cell is achieved by administration of:
(i) an activator of a SIRT selected from: a 1,4-dihydropyridine (DHP) derivative bearing a benzyl group at the N1 position, SRT1720 HCl, and Fisetin; or
(ii) an inhibitor of NLRP3 selected from: glyburide, 16673-34-0, MCC950, Shikonin, sodium butyrate, β-hydroxybutyrate, and an siRNA targeting NLRP3 mRNA;
(iii) an inhibitor of caspase-1 selected from: z-VAD-fmk, ac-YVAD-cmk, VX-765, and z-WEHD-fmk; or
(iv) an agent that increases intracellular NAD⁺ levels selected from: a P2X7R receptor, extracellular NADH, CoQ10, LDH, G3PDH, MDH, NMN and NR.

21. A method of reducing tissue degeneration in an individual, the method comprising administering to the individual an agent that:
a) increases the level of a SIRT2 polypeptide in the adult stem cell;
b) increases the deacetylase activity of a SIRT2 polypeptide in the adult stem cell;
c) increases deacetylation of a nucleotide-binding domain and leucine-rich repeat-containing-3 (NLRP3) polypeptide in the adult stem cell;
d) reduces the level and/or activity of an NLRP3 polypeptide in the adult stem cell;
e) reduces the level and/or activity of a caspase-1 polypeptide in the adult stem cell; or
f) inhibits pyroptosis in the adult stem cell.

22. A method of treating or ameliorating a tissue degenerative disease, including neurodegenerative diseases (e.g., Alzheimer's Disease), muscle degenerative diseases (e.g., muscular dystrophy), and bone marrow failure, in an individual, the method comprising administering to the individual an agent that:
a) increases the level of a SIRT2 polypeptide in the adult stem cell;
b) increases the deacetylase activity of a SIRT2 polypeptide in the adult stem cell;
c) increases deacetylation of a nucleotide-binding domain and leucine-rich repeat-containing-3 (NLRP3) polypeptide in the adult stem cell;
d) reduces the level and/or activity of an NLRP3 polypeptide in the adult stem cell;
e) reduces the level and/or activity of a caspase-1 polypeptide in the adult stem cell; or
f) inhibits pyroptosis in the adult stem cell.

23. The method of claim 21 or claim 22, wherein the agent is:
(i) an activator of a SIRT selected from: a 1,4-dihydropyridine (DHP) derivative bearing a benzyl group at the N1 position, SRT1720 HCl, and Fisetin; or
(ii) an inhibitor of NLRP3 selected from: glyburide, 16673-34-0, MCC950, Shikonin, sodium butyrate, β-hydroxybutyrate, and an siRNA targeting NLRP3 mRNA;
(iii) an inhibitor of caspase-1 selected from: z-VAD-fmk, ac-YVAD-cmk, VX-765, and z-WEHD-fmk; or
(iv) an agent that increases intracellular NAD⁺ levels selected from: a P2X7R receptor, extracellular NADH, CoQ10, LDH, G3PDH, MDH, NMN and NR.


SEQUENCE LISTING (54 sequences)

SEQ ID NO: 1 - protein, 11 aa, Artificial Sequence
  Synthetic Sequence
YGRKKRRQRRR

SEQ ID NO: 2 - protein, 12 aa, Artificial Sequence
  Synthetic Sequence
RRQRRTSKLMKR

SEQ ID NO: 3 - protein, 27 aa, Artificial Sequence
  Synthetic Sequence
GWTLNSAGYLLGKINLKALAALAKKIL

SEQ ID NO: 4 - protein, 33 aa, Artificial Sequence
  Synthetic Sequence
KALAWEAKLAKALAKALAKHLAKALAKALKCEA

SEQ ID NO: 5 - protein, 16 aa, Artificial Sequence
  Synthetic Sequence
RQIKIWFQNRRMKWKK

SEQ ID NO: 6 - protein, 9 aa, Artificial Sequence
  Synthetic Sequence
RKKRRQRRR

SEQ ID NO: 7 - protein, 8 aa, Artificial Sequence
  Synthetic Sequence
RKKRRQRR

SEQ ID NO: 8 - protein, 11 aa, Artificial Sequence
  Synthetic Sequence
YARAAARQARA

SEQ ID NO: 9 - protein, 11 aa, Artificial Sequence
  Synthetic Sequence
THRLPRRRRRR

SEQ ID NO: 10 - protein, 11 aa, Artificial Sequence
  Synthetic Sequence
GGRRARRRRRR

SEQ ID NO: 11 - protein, 16 aa, Artificial Sequence
  Synthetic Sequence
LKKFKMHLEDYPPEKG

SEQ ID NO: 12 - protein, 22 aa, Artificial Sequence
  Synthetic Sequence
KLAQYLEDLEDVDLKKFKMHLE

SEQ ID NO: 13 - protein, 26 aa, Artificial Sequence
  Synthetic Sequence
FQGAAGIGKTILARKIMLDWALGKLF

SEQ ID NO: 14 - protein, 14 aa, Artificial Sequence
  Synthetic Sequence
KDKFDYLFFIHCRE

SEQ ID NO: 15 - protein, 14 aa, Artificial Sequence
  Synthetic Sequence
RKGPGGCSDLLNRD

SEQ ID NO: 16 - protein, 21 aa, Artificial Sequence
  Synthetic Sequence
YLEKKLSCKISQQVRLELLKW

SEQ ID NO: 17 - protein, 17 aa, Artificial Sequence
  Synthetic Sequence
YLEKKLSCKISQQVRLE

SEQ ID NO: 18 - protein, 29 aa, Artificial Sequence
  Synthetic Sequence
RLYIGENALGDSGVQVLCEKMKDPQCNLQ

SEQ ID NO: 19 - protein, 28 aa, Artificial Sequence
  Synthetic Sequence
LYIGENALGDSGVQVLCEKMKDPQCNLQ

SEQ ID NO: 20 - protein, 28 aa, Artificial Sequence
  Synthetic Sequence
RLYIGENALGDSGVQVLCEKMKDPQCNL

SEQ ID NO: 21 - protein, 24 aa, Artificial Sequence
  Synthetic Sequence
RKLNLGNNDLGDLCVVTLCEVLKQ

SEQ ID NO: 22 - protein, 829 aa, Mus musculus
MTSVRCKLAQYLEDLEDVDLKKFKMHLEDYPPEKGCIPVPRGQMEKADHLDLATLMIDFNGEEKAWAMAVWIFAAINRRDLWEKAKKDQPEWNDTCTSHSSMVCQEDSLEEEWMGLLGYLSRISICKKKKDYCKMYRRHVRSRFYSIKDRNARLGESVDLNSRYTQLQLVKEHPSKQEREHELLTIGRTKMRDSPMSSLKLELLFEPEDGHSEPVHTVVFQGAAGIGKTILARKIMLDWALGKLFKDKFDYLFFIHCREVSLRTPRSLADLIVSCWPDPNPPVCKILRKPSRILFLMDGFDELQGAFDEHIGEVCTDWQKAVRGDILLSSLIRKKLLPKASLLITTRPVALEKLQHLLDHPRHVEILGFSEAKRKEYFFKYFSNELQAREAFRLIQENEVLFTMCFIPLVCWIVCTGLKQQMETGKSLAQTSKTTTAVYVFFLSSLLQSRGGIEEHLFSDYLQGLCSLAADGIWNQKILFEECDLRKHGLQKTDVSAFLRMNVFQKEVDCERFYSFSHMTFQEFFAAMYYLLEEEAEGETVRKGPGGCSDLLNRDVKVLLENYGKFEKGYLIFVVRFLFGLVNQERTSYLEKKLSCKISQQVRLELLKWIEVKAKAKKLQWQPSQLELFYCLYEMQEEDFVQSAMDHFPKIEINLSTRMDHVVSSFCIKNCHRVKTLSLGFFHNSPKEEEEERRGGRPLDQVQCVFPDTHVACSSRLVNCCLTSSFCRGLFSSLSTNRSLTELDLSDNTLGDPGMRVLCEALQHPGCNIQRLWLGRCGLSHQCCFDISSVLSSSQKLVELDLSDNALGDFGIRLLCVGLKHLLCNLQKL

SEQ ID NO: 23 - protein, 1016 aa, Homo sapiens
MKMASTRCKLARYLEDLEDVDLKKFKMHLEDYPPQKGCIPLPRGQTEKADHVDLATLMIDFNGEEKAWAMAVWIFAAINRRDLYEKAKRDEPKWGSDNARVSNPTVICQEDSIEEEWMGLLEYLSRISICKMKKDYRKKYRKYVRSRFQCIEDRNARLGESVSLNKRYTRLRLIKEHRSQQEREQELLAIGKTKTCESPVSPIKMELLFDPDDEHSEPVHTVVFQGAAGIGKTILARKMMLDWASGTLYQDRFDYLFYIHCREVSLVTQRSLGDLIMSCCPDPNPPIHKIVRKPSRILFLMDGFDELQGAFDEHIGPLCTDWQKAERGDILLSSLIRKKLLPEASLLITTRPVALEKLQHLLDHPRHVEILGFSEAKRKEYFFKYFSDEAQARAAFSLIQENEVLFTMCFIPLVCWIVCTGLKQQMESGKSLAQTSKTTTAVYVFFLSSLLQPRGGSQEHGLCAHLWGLCSLAADGIWNQKILFEESDLRNHGLQKADVSAFLRMNLFQKEVDCEKFYSFIHMTFQEFFAAMYYLLEEEKEGRTNVPGSRLKLPSRDVTVLLENYGKFEKGYLIFVVRFLFGLVNQERTSYLEKKLSCKISQQIRLELLKWIEVKAKAKKLQIQPSQLELFYCLYEMQEEDFVQRAMDYFPKIEINLSTRMDHMVSSFCIENCHRVESLSLGFLHNMPKEEEEEEKEGRHLDMVQCVLPSSSHAACSHGLVNSHLTSSFCRGLFSVLSTSQSLTELDLSDNSLGDPGMRVLCETLQHPGCNIRRLCNQKLVELDLSDNALGDFGIRLLCVGLKHLLCNLKKLWLVSCCLTSACCQDLASVLSTSHSLTRLYVGENALGDSGVAILCEKAKNPQCNLQKLGLVNSGLTSVCCSALSSVLSTNQNLTHLYLRGNTLGDKGIKLLCEGLLHPDCKLQVLELDNCNLTSHCCWDLSTLLTSSQSLRKLSLGNNDLGDLGVMMFCEVLKQQSCLLQNLGLSEMYFNYETKSALETLQEEKPELTVVFEPSW

SEQ ID NO: 24 - protein, 1035 aa, Macaca mulatta
MKMMSVRCKLAQYLEDLEDVDLKKFKMHLEDYPPEKGCVPIPRGQMEKADHLDLATLMIDFNGEEKAWGMAVWIFAAINRRDLWEKAKKDQPEWNDACTSNLSMVCQEDSLEEEWIGLLGYLSRISICKKKKDYCKIYRRHVRSRFYSIKDRNARLGESVDLNRRYTQLQLVKEHPSKQEREHELLTIGRTKMWDRPMSSLKLELLFEPEDEHLEPVHTVVFQGAAGIGKTILARKIMLDWALGKLFKDKFDYLFFIHCREVSLRAPKSLADLIISCWPDPNPPVCKILCKPSRILFLMDGFDELQGAFDEHIEEVCTDWQKAVRGDILLSSLIRKKLLPKASLLITTRPVALEKLQHLLDHPRHVEILGFSEAKRKEYFFKYFSNELQAREAFRLIQENEILFTMCFIPLVCWIVCTGLKQQMETGKSLAQTSKTTTAVYVFFLSSLLQSRGGIEEHLFSAYLPGLCSLAADGIWNQKILFEECDLRKHGLQKTDVSAFLRMNVFQKEVDCERFYSFSHMTFQEFFAAMYYLLEEEEEGVTVRKGPEGCSDLLNRDVKVLLENYGKFEKGYLIFVVRFLFGLVNQERTSYLEKKLSCKISQQVRLELLKWIEVKAKAKKLQRQPSQLELFYCLYEMQEEDFVQSAMGHFPKIEINLSTRMDHVVSSFCIKNCHRVKTLSLGFLHNSPKEEEEEKRGSQPLDQVQCVFPDPHVACSSRLVNCCLTSSFCRGLFSSLSTNQSLTELDLSDNTLGDPGMRVLCEALQHPGCNIQRLWLGRCGLTHQCCFNISSVLSSSQKLVELDLSDNALGDFGVRLLCVGLKHLLCNLQKLWLVSCCLTSACCQDLALVLSSNHSLTRLYIGENALGDSGVQVLCEKMKDPQCNLQKLGLVNSGLTSLCCSALTSVLKTNQNLTHLYLRSNALGDMGLKLLCEGLLHPDCKLQMLELDNCSLTSHSCWDLSTILTHNQSLRKLNLSNNDLGDLCVVTLCEVLKQQGCLLQSLQLGEMYLNCETKRTLEALQEEKPELTVVFEISW

SEQ ID NO: 25 - protein, 1031 aa, Bos taurus
MRMVSVRCKLARYLEDLEDIDFKKFKMHLEDYPSQKGCTSIPRGQTEKADHVDLATLMIDFNGEEKAWAMAKWIFAAINRRDLYEKAKREEPEWENANISVLSQEESLEEEWMGLLGYLSRISICRKKKDYCKKYRKYVRSKFQCIKDRNARLGESVNLNKRFTRLRLIKEHRSQQEREHELLAIGRTWAKIQDSPVSSVNLELLFDPEDQHSEPVHTVVFQGAAGIGKTILARKIMLDWASEKLYQDRFDYLFYIHCREVSLGTQRSLGDLIASCCPGPNPPIGKIVSKPSRILFLMDGFDELQGAFDEHTEALCTNWRKVERGDILLSSLIRKRLLPEASLLITTRPVALEKLQHLLGQARHVEILGFSEARRKEYFLKYFSDEQQAREAFRLIQENEILFTMCFIPLVCWIVCTGLKQQMDSGKSLARTSKTTTAVYIFFLSSLLQSQGGSQENHNSATLWGLCSLAADGIWNQKILFQECDLRNHGLQKADVSAFLRMNLFQKEVDCEKFYSFIHMTFQEFFAAMYYLLEEDNHGEMRNTPQACSKLPNRDVKVLLENYGKFEKGYLIFVVRFLFGLINQERTSYLEKKLSCKISQKIRLELLKWIEAKANAKTLQIEPSQLELFYCLYEMQEEDFVQRAMSHFPKIEIKLSTRMDHVVSSFCIENCRHVESLSLRLLHNSPKEEEEEEEVRHSHMDRSVLSDFEVAYSQGLVNYLTSSICRGIFSVLSNNWNLTELNLSGNTLGDPGMNVLCETLQQPGCNIRRLWLGQCCLSHQCCFNISSVLSNNQKLVELDLSHNALGDFGIRLLCVGLRHLFCNLKKLWLVSCCLTSASCEDLASVLSTNHSLTRLYLGENALGDSGVGILCEKVKNPHCNLQKLGLVNSGLTSGCCPALSSVLSTNQNLTHLYLQGNALGDMGVKLLCEGLLHRNCKLQVLELDNCSLTSHCCWDLSTLLTSNQSLRKLCLGNNDLGDLGVMLLCEVLKQQGCLLKSLRLCEMYFNYDTKRALETLQEEKPELTIVFEPSR

SEQ ID NO: 26 - protein, 1027 aa, Equus caballus
MASVRCKLARYLEDLEDADLKKFKMHLEDYPPQKGCTRPPRGQTEKADPVDLATLMIDFNGEEKAWAMAVWIFAAINRRDLHEKAKLEEPEWAHSCCYRSHLICQEESIEEEWIGLLEYLSRISICKKKKDYCKKYRKHVRSRFQCIKDRNARLGESVNLNKRYTRLCLLKEHRSQQEREQELLAIGRTSPKTQDCPMSSMNLELLFEPDDQHSEPVHTVVFQGAAGIGKTILARKIMLDWASEKLYKNRFDYLFYIHCREVSLGTRRSLGDLIVSCCPDPKPPIGKILSKPSRILFLMDGFDELQGAFDEHTEALCTDWQKVERGDILLSSLIRKRLLPEASLLITTRPVALEKLQHLLDRPRHVEILGFSEAKRKEYFFKYFSDEQQAHEAFRLIQENEILFTMCFIPLVCWIVCTGLKQQMDSGKSLAQTSKTTTAVYVFFLSSLLQSRGESQKQHLSANVRGLCSLAADGIWNQKILFEESDLRKHGLQRADASAFLRMNLFQKEVDCEKFYSFIHMTFQEFFAAMYYLLEDEEQGSVRNMPWGRSNLPNRDVSVLLENYGKFEKGYLIFVVRFLFGLVNQERTSYLEKKLSCKISQQIRLELLKWISAKAEVKKLQTQPSQLELFYCLYEMQEEDFVRRAMGYFPKIEISLSTRMDHVVSSFCIENCRSVESLSLRLLYNSPKEEEEEEEEEEDGIDFLFSPSSFLPISRLVNCSLTSSFCQSLFSALSTNWSLTELNLGDNTLGDSGMKVLCDMLQHPGCNIQRLCATHWGLKQQKFTFSHLITSLKALSPNTVTFSGCGETGTTLHCGWKCKMVQLLLVGCCLTSACCEDLASVLSTSHSLIRLYLGENALGDSGVGILCEKAKHPRCNLQVLGLVNSGLTSGCCPALSSVLSANHNLKQLYLRGNALGDTGVKLLCEGLSHPNCKLQMLELDSCSLTSHCCWDLSTLLTSNQSLRELSLGGNDLGDLGVMLLCEVLKQQGCLLRSLGLCEMYFNYETKCALETLQEEKPELSVVFEPSW

SEQ ID NO: 27 - protein, 722 aa, Sus scrofa
MSMASVRCKLARYLEDLEDVDFKKFKMHLEDYPSQKGCISLPRGQTEKADHVDLATLMIDFNGEEKAWAMATWIFAAINRRDLYEKAKRDEPEWVPDNANLSVISQEESLEEEWMGLLGYLSRISICKKKKDYCKKYRKHVRSRFQCIKDRNARLGESVNLNKRFTRLRLIKEHRSQQEREHELLAIGRTSAKMQDGPVSSLNLELLFDPEDQHSEPVHTVVFQGAAGIGKTILARKIMLDWASEKLYQEKFDYLFYIHCREVSLGTRRSLGDLIASCCPGPNPPIGKIVSKPSRILFLMDGFDELQGAFDERTEALCTNWQKVERGDILLSSLIRKRLLPEASLLITTRPVALEKLQHLLGRARHVEILGFSEAKRKEYFFKYFSDEQQAREAFRLIQENEVLFTMCFIPLVCWIVCTGLKQQMDSGKSLARTSKTTTAVYIFFLSTLLQSQEGSQDHHVSATLWGLCSLAADGIWNQKILFEECDLRNHGLQKADVSAFLRMNLFQKEVDCEKFYSFIHMTFQEFFAAMYYLLEKEDQGEMRNVPRSCLKLPSRDVTVLLENYGKFEKGYLIFVVRFLFGLVNQERTSYLEKKLSCKISQKIRLELLKWIEAKAKAKKLQIEPSQLELFYCLYEMQEEDFVQKAMGHFPKIEINLSTRMDHVVSSFCIENCRHVESLSLRLLHNSPKEEEEEEEIEEEDVQHSDVDNCILSESHVAYSQR

SEQ ID NO: 28 - protein, 1035 aa, Rattus norvegicus; identical to SEQ ID NO: 24

SEQ ID NO: 29 - protein, 1046 aa, Oryctolagus cuniculu
MAGTRCKLAQYLEDLEELDLKKFKMHLEDYPPDQGCIPLPRGQAARADPVDLATLMIDFNGEEKAWAMALWIFTAINRRDLYERAKRDEPVWEPAHVSNATVICHEEIIEEERMGILGHLSRISICTKRKDYCKKYRKHVRSRFQCIEDRNARLGESVNLNKRYTRLRLAKEHLSRQEREQELVAIGRTHVWDSPASPVRVELLFDADEECLEPVRTVVLQGAAGIGKTILARKIMLDWASGQLFQDRFDYLFYIHCREMSLGMHRSLVDLIAGCCSDPSPPVSKMLHRPSRVLFLMDGFDELQGAFDESSGALCSDWRVAVRGDVLLGSLIRKKLLPEASLLITTRPVALEKLQHLLDRPRHVEVLGFSEARRKEYFFKYFLEEAQAREAFSLLQENEVLLSMCCIPLVCWIVCTGLKQQMQGGKHRAKTTTSKTTTAVYTFFLSSLLQSRGGSQEQRLSGHLRGLCSLAADGIWNQKILFEERDLRRHGLQEAEVSAFLRMSLFQKEVDCEAFYSFVHLTFQEFFAAMYYVLEEEEEEKEEEEGAGTGRSPPARGLDLPHRDVRVLLENYGKFEKGYLIFVVRFLFGLVNQERASYLEKKLSCKISQQVRPELLRWIETKANAKKLQIQPSQLELFYCLYEMQEEAFVREAMGHFPRIEINLSTRMDHVVSSFCIGNCHSVESLSLGFLHNSPKEEEEEEEEEEVESPLFDVGCPNRQGPHAACSWRLVNSCLTSSFCRGLFSVLSTNPSLTELDLSDNSLGDPGMRVLCEALQHPGCNIRRLWLGRCGLSHQCCFDISLVLSGSQKLVELDLSDNALGDFGVRLLCVGLRHLSCQLQKLWLVSCCLTSMCCQDLASVLSTNHCLTRLYLGENTLGDYGVGLLCEKVKHPQCNLQKLGLVNSGLTSACCAALSAALSTSRSLTHLYLRGNALGDAGVKLLCEGLLHPSCRLQMLELDSCSLTSHCCWNLCTILTCSRSLRKLSLGNNDLGDLGVMMLCEVLRQPDCPLQRLQLCEMYFNPETIHALETLREEKPALTVVFEPSW

SEQ ID NO: 30 - protein, 19 aa, Artificial Sequence
  Synthetic Sequence
TGGGCTGGATGAAAGAGAA

SEQ ID NO: 31 - protein, 21 aa, Artificial Sequence
  Synthetic Sequence
GGTCCACCTTGGAGAAGTCTG

SEQ ID NO: 32 - protein, 18 aa, Artificial Sequence
  Synthetic Sequence
GCGGAGGTGGGAGATATG

SEQ ID NO: 33 - protein, 20 aa, Artificial Sequence
  Synthetic Sequence
CGTAGAAGGTTTGGAACAGC

SEQ ID NO: 34 - protein, 20 aa, Artificial Sequence
  Synthetic Sequence
GATCTGGCACCACACCTTCT

SEQ ID NO: 35 - protein, 20 aa, Artificial Sequence
  Synthetic Sequence
GGGGTGTTGAAGGTCTCAAA

SEQ ID NO: 36 - protein, 20 aa, Artificial Sequence
  Synthetic Sequence
GCAACAGCATCTTGCCTGAT

SEQ ID NO: 37 - protein, 18 aa, Artificial Sequence
  Synthetic Sequence
GTGCTACTGGTCTCACTT

SEQ ID NO: 38 - protein, 979 aa, Homo sapiens
MKMASTRCKLARYLEDLEDVDLKKFKMHLEDYPPQKGCIPLPRGQTEKADHVDLATLMIDFNGEEKAWAMAVWIFAAINRRDLYEKAKRDEPKWGSDNARVSNPTVICQEDSIEEEWMGLLEYLSRISICKMKKDYRKKYRKYVRSRFQCIEDRNARLGESVSLNKRYTRLRLIKEHRSQQEREQELLAIGKTKTCESPVSPIKMELLFDPDDEHSEPVHTVVFQGAAGIGKTILARKMMLDWASGTLYQDRFDYLFYIHCREVSLVTQRSLGDLIMSCCPDPNPPIHKIVRKPSRILFLMDGFDELQGAFDEHIGPLCTDWQKAERGDILLSSLIRKKLLPEASLLITTRPVALEKLQHLLDHPRHVEILGFSEAKRKEYFFKYFSDEAQARAAFSLIQENEVLFTMCFIPLVCWIVCTGLKQQMESGKSLAQTSKTTTAVYVFFLSSLLQPRGGSQEHGLCAHLWGLCSLAADGIWNQKILFEESDLRNHGLQKADVSAFLRMNLFQKEVDCEKFYSFIHMTFQEFFAAMYYLLEEEKEGRTNVPGSRLKLPSRDVTVLLENYGKFEKGYLIFVVRFLFGLVNQERTSYLEKKLSCKISQQIRLELLKWIEVKAKAKKLQIQPSQLELFYCLYEMQEEDFVQRAMDYFPKIEINLSTRMDHMVSSFCIENCHRVESLSLGFLHNMPKEEEEEEKEGRHLDMVQCVLPSSSHAACSHGLGRCGLSHECCFDISLVLSSNQKLVELDLSDNALGDFGIRLLCVGLKHLLCNLKKLWLVSCCLTSACCQDLASVLSTSHSLTRLYVGENALGDSGVAILCEKAKNPQCNLQKLGLVNSGLTSVCCSALSSVLSTNQNLTHLYLRGNTLGDKGIKLLCEGLLHPDCKLQVLELDNCNLTSHCCWDLSTLLTSSQSLRKLSLGNNDLGDLGVMMFCEVLKQQSCLLQNLGLSEMYFNYETKSALETLQEEKPELTVVFEPSW

SEQ ID NO: 39 - protein, 352 aa, Homo sapiens
MDFLRNLFSQTLSLGSQKERLLDELTLEGVARYMQSERCRRVICLVGAGISTSAGIPDFRSPSTGLYDNLEKYHLPYPEAIFEISYFKKHPEPFFALAKELYPGQFKPTICHYFMRLLKDKGLLLRCYTQNIDTLERIAGLEQEDLVEAHGTFYTSHCVSASCRHEYPLSWMKEKIFSEVTPKCEDCQSLVKPDIVFFGESLPARFFSCMQSDFLKVDLLLVMGTSLQVQPFASLISKAPLSTPRLLINKEKAGQSDPFLGMIMGLGGGMDFDSKKAYRDVAWLGECDQGCLALAELLGWKKELEDLVRREHASIDAQSGAGVPNPSTSASPKKSPPPAKDEARTTEREKPQ

SEQ ID NO: 40 - protein, 404 aa, Homo sapiens
MADKVLKEKRKLFIRSMGEGTINGLLDELLQTRVLNKEEMEKVKRENATVMDKTRALIDSVIPKGAQACQICITYICEEDSYLAGTLGLSADQTSGNYLNMQDSQGVLSSFPAPQAVQDNPAMPTSSGSEGNVKLCSLEEAQRIWKQKSAEIYPIMDKSSRTRLALIICNEEFDSIPRRTGAEVDITGMTMLLQNLGYSVDVKKNLTASDMTTELEAFAHRPEHKTSDSTFLVFMSHGIREGICGKKHSEQVPDILQLNAIFNMLNTKNCPSLKDKPKVIIIQACRGDSPGVVWFKDSVGVSGNLSLPTTEEFEDDAIKKAHIEKDFIAFCSSTPDNVSWRHPTMGSVFIGRLIEHMQEYACSCDVEEIFRKVRFSFEQPDGRAQMPTTERVTLTRCFYLFPGH

SEQ ID NO: 41 - protein, 383 aa, Homo sapiens
MADKVLKEKRKLFIRSMGEGTINGLLDELLQTRVLNKEEMEKVKRENATVMDKTRALIDSVIPKGAQACQICITYICEEDSYLAGTLGLSAAPQAVQDNPAMPTSSGSEGNVKLCSLEEAQRIWKQKSAEIYPIMDKSSRTRLALIICNEEFDSIPRRTGAEVDITGMTMLLQNLGYSVDVKKNLTASDMTTELEAFAHRPEHKTSDSTFLVFMSHGIREGICGKKHSEQVPDILQLNAIFNMLNTKNCPSLKDKPKVIIIQACRGDSPGVVWFKDSVGVSGNLSLPTTEEFEDDAIKKAHIEKDFIAFCSSTPDNVSWRHPTMGSVFIGRLIEHMQEYACSCDVEEIFRKVRFSFEQPDGRAQMPTTERVTLTRCFYLFPGH

SEQ ID NO: 42 - protein, 311 aa, Homo sapiens
MADKVLKEKRKLFIRSMGEAPQAVQDNPAMPTSSGSEGNVKLCSLEEAQRIWKQKSAEIYPIMDKSSRTRLALIICNEEFDSIPRRTGAEVDITGMTMLLQNLGYSVDVKKNLTASDMTTELEAFAHRPEHKTSDSTFLVFMSHGIREGICGKKHSEQVPDILQLNAIFNMLNTKNCPSLKDKPKVIIIQACRGDSPGVVWFKDSVGVSGNLSLPTTEEFEDDAIKKAHIEKDFIAFCSSTPDNVSWRHPTMGSVFIGRLIEHMQEYACSCDVEEIFRKVRFSFEQPDGRAQMPTTERVTLTRCFYLFPGH

SEQ ID NO: 43 - protein, 263 aa, Homo sapiens
MADKVLKEKRKLFIRSMGEAPQAVQDNPAMPTSSGSEGNVKLCSLEEAQRIWKQKSAEIYPIMDKSSRTRLALIICNEEFDSIPRRTGAEVDITGMTMLLQNLGYSVDVKKNLTASDMTTELEAFAHRPEHKTSDSTFLVFMSHGIREGICGKKHSEQVPDILQLNAIFNMLNTKNCPSLKDKPKVIIIQACRGDNVSWRHPTMGSVFIGRLIEHMQEYACSCDVEEIFRKVRFSFEQPDGRAQMPTTERVTLTRCFYLFPGH

SEQ ID NO: 44 - protein, 88 aa, Homo sapiens
MADKVLKEKRKLFIRSMGEDNVSWRHPTMGSVFIGRLIEHMQEYACSCDVEEIFRKVRFSFEQPDGRAQMPTTERVTLTRCFYLFPGH

SEQ ID NO: 45 - DNA, 21 bp, Artificial Sequence
  Synthetic sequence
ccagaataaggcatttctcta

SEQ ID NO: 46 - DNA, 21 bp, Artificial Sequence
  Synthetic Sequence
aagcggcttgagggtaatcaa

SEQ ID NO: 47 - DNA, 57 bp, Artificial Sequence
  Synthetic Sequence
attccacgtcttgccctcattattcaagagataatgagggcaagacgtgtttttttg

SEQ ID NO: 48 - DNA, 58 bp, Artificial Sequence
  Synthetic Sequence
gatccaaaaaaacacgtcttgccctcattatctcttgaataatgagggcaagacgtgg

SEQ ID NO: 49 - DNA, 54 bp, Artificial Sequence
  Synthetic Sequence
attcggacaataaatggattgttggtcaagagccaacaatccatttattgtccg

SEQ ID NO: 50 - DNA, 55 bp, Artificial Sequence
  Synthetic Sequence
gatccggacaataaatggattgttggctcttgaccaacaatccatttattgtccg

SEQ ID NO: 51 - DNA, 60 bp, Artificial Sequence
  Synthetic Sequence
attcccacatgactttccaggagtttcaagagaactcctggaaagtcatgtggttttttg

SEQ ID NO: 52 - DNA, 61 bp, Artificial Sequence
  Synthetic Sequence
gatccaaaaaaccacatgactttccaggagttctcttgaaactcctggaaagtcatgtggg

SEQ ID NO: 53 - DNA, 60 bp, Artificial Sequence
  Synthetic Sequence
attcgggtgaagatatcgacataattcaagagattatgtcgatatcttcacccttttttg

SEQ ID NO: 54 - DNA, 61 bp, Artificial Sequence
  Synthetic Sequence
gatccaaaaaagggtgaagatatcgacataatctcttgaattatgtcgatatcttcacccg